(19) United States
(12) Patent Application Publication
(10) Pub. No.: US 2011/0159121 A1
(43) Pub. Date: Jun. 30, 2011

(54) METHODS AND COMPOSITIONS FOR IDENTIFYING, PRODUCING AND USING PLANT-DERIVED PRODUCTS FOR MODULATING CELL FUNCTION AND AGING

(75) Inventor: David H. McDaniel, Virginia Beach, VA (US)

(73) Assignee: LifeSpan Extension, LLC

(21) Appl. No.: 12/976,741

(22) Filed: Dec. 22, 2010

Related U.S. Application Data
(60) Provisional application No. 61/290,149, filed on Dec. 24, 2009.

(30) Foreign Application Priority Data
Dec. 22, 2010 (US) PCT/US2010/061849

(51) Int. Cl. A61K 36/889; C12Q 1/68; C12Q 1/02; C12N 5/00; A61P 39/00; A61P 35/00; A61K 36/45; A61K 36/13; A61K 36/87; A61K 36/63; A61K 36/73; A61K 36/82

(52) U.S. Cl. 424/727; 435/6.13; 435/29; 435/375; 424/732; 424/770; 424/766; 424/769; 424/765; 424/729

(57) ABSTRACT

Provided herein are methods of culturing cells in vitro in order to exploit the biochemical production ability of the cells to make metabolites that are evaluated and harvested for their biological effects. Also provided are systems for evaluating extracts from such cultured cells to characterize their biological activity(s), particularly with regard to impact on health, wellbeing, longevity, DNA maintenance, mitochondrial health and/or biogenesis, and so forth. Biologically active extracts, components thereof, and compositions (such as cosmetic or pharmaceutical preparations) made comprising such, are also provided.

### CROSS REFERENCE TO RELATED APPLICATION

This application claims priority to International Application No. PCT/US2010/061849 and also claims the benefit of the earlier filing date of U.S. Provisional Application No. 61/290,149, filed Dec. 24, 2009. Both applications are hereby incorporated by reference in their entirety.

### FIELD

This disclosure relates to methods of identifying, purifying, and using non-animal or non-animal-derived compositions useful in modulating lifespan, cell function and aging. Optionally, the compositions are produced by and/or from cultured (e.g., tissue or suspension cultured) plant, algae, fungus, or bacterial cells; optionally, such cultured cells are subject to one or more types of elicitation prior to preparation of the composition. Representative compositions are useful to directly or indirectly reduce damage (e.g., oxidative damage) to DNA in cells, to directly or indirectly modulate telomere maintenance, mitochondrial biogenesis and/or respiration, and so forth. Also described are systems for decreasing lifespan of cells, for instance deleterious cells such as precancerous and cancerous cells.

### BACKGROUND

All living cells and organisms have a finite lifespan. They live for a period of time and die. Cells and organisms have both a chronological age and a biological age. The former is measured in days, months or years while the latter may be measured by a host of complex testing of biological functions including but not limited to: gene expression, protein production or metabolic pathways. The rate of aging may also be measured, and an accelerated rate of aging may be considered ‘premature aging’, while a slower rate of aging may extend lifespan. It is desirable to maximize the healthy lifespan of cells and organisms and it is also desirable to extend the healthy lifespan by delaying the rate of aging and the onset of dysfunctional or disease states. Shortening the lifespan and/or accelerating apoptosis of unhealthy, diseased, damaged, or cancerous cells may also be desirable.

Oxidative stress is one of the primary causes of cell and organism dysfunction or disease and also accelerated or premature aging and death. The ability to enhance in a favorable manner the ability of cells and organisms to resist or repair damage due to oxidative stress produced by environmental injury, lifestyle choices as well as diseases and medical therapies may extend the healthy function and/or lifespan and/or retard aging and senescence. Antioxidants have the potential not only to neutralize reactive oxygen species, but also may provide vital anti-aging benefits by affecting various other key cellular mechanisms. One such example is the telomere (and/or telomere unit and associated proteins and structural configurations) which are special chromatin structures at the end of chromosomes. Premature or accelerated telomere shortening may produce premature aging and death. Telomerase is a DNA polymerase which plays an essential role in protecting these regions, but which may also be associated with cancer. Thus the ability to modulate telomerase activity provides the opportunity to alter health both positively and negatively.

One way to extend the lifespan of a living cell—and by extension possibly the organ, tissue or entire organism—is to repair damage in addition to preventing damage. The genes which control the cellular repair mechanisms, if activated or enhanced in the proper way, may effectively extend the lifespan of a cell. This may take several forms: extending the lifespan of a cell which is damaged or injured by properly repairing that damage and/or by causing the cell to live longer or replicate itself longer than it would have occurred naturally.

Mammalian mitochondria are organelles that produce more than 90% of cellular ATP under aerobic conditions through a process called oxidative phosphorylation. Mitochondria are also involved in fatty acid metabolism, hormone production, ketone body production, apoptosis, and Ca²⁺ homeostasis. Mitochondria contain, inter alia, the TCA cycle (also known as the Kreb cycle), enzymes involved in heme biosynthesis and the electron transport chain (OXPHOS system). Due to the large flux of redox reactions necessary to maintain oxidative phosphorylation, the organelle is the site of production of reactive oxygen species (ROS), which in controlled production have a signaling function, but in overproduction are toxic and are believed to be the cause of many human diseases including, for example, Parkinson's disease and other neurodegenerative conditions, diabetes, and the aging process itself.

Oxidative stress is one of the primary causes of cell and organism dysfunction or disease and also accelerated or premature aging and death. Mitochondrial function or dysfunction, biogenesis, death and regenesis also play a vital role in the aging process. The ability to enhance in a favorable manner the ability of cells and organisms to resist or repair damage due to oxidative stress produced by environmental injury, lifestyle choices as well as diseases and medical therapies may extend the healthy function and/or lifespan and/or retard aging and senescence.

The ability to extend or prolong lifespan (both healthy and less healthy) lies in the ability to extend the lifespan of cells, both differentiated specialized cells and also undifferentiated stem and progenitor cells so that cell lifespan is longer or so that new cells replace senescent cells which lose their function or die. A cell normally has a finite lifespan determined by the number of cell divisions which are possible. The Hayflick Limit theory discusses one view of lifespan limitations. An organ may be repopulated with cells to regenerate itself from the stem cell population but the stem and progenitor cells themselves have a finite lifespan. The ability to extend the lifespan of differentiated cells and/or stem and progenitor cells lies at the heart of extending lifespan of an organism.

The molecular reduction of oxygen to water during oxidative phosphorylation results inevitably in the production of superoxide radicals (O₂^{•-}) that are reactive oxygen species containing an unpaired electron orbital. Superoxides act as either reductants or oxidants and can form other reactive species including the hydroxyl radical (OH⁻) through interaction with iron (Haber-Weiss reaction) and peroxynitrite by reaction with nitric oxide. Reactive oxygen species attack proteins, DNA, and membrane lipids, thereby disrupting cellular function and integrity.

It has long been believed that oxidative damage to cells, tissues, and genetic material, plays a major role in aging and illness. Sources of oxidative damage are many, and include chemicals present in the environment, aging, disease, intense exercise, and ionizing radiation. Additionally, many products and byproducts of cellular metabolism can cause or contribute to oxidative damage.

Even though mammals produce a number of antioxidant enzymes, these enzymes are often insufficient to adequately eliminate oxidative agents; conditions of heightened oxidative stress only make matters worse. Dietary supplementation with antioxidants can be particularly useful in lessening the damage caused by any oxidative agents.

Plants have evolved with enhanced secondary metabolism systems (production of secondary metabolites or phytochemicals), including for instance the phenylpropanoid pathway, that provide compounds useful in defense of the plants against environmental influences and pathogen attacks. These phenylpropanoid compounds are often present in plants that animals have selected for food, and people have for generations influenced the levels and amounts of secondary metabolites in plants through selection and selective breeding—and more recently through genetic engineering. Selection criteria have included perceived beneficial characteristics of (for instance) grains, fruits, flowers and vegetables—including ease of cultivation, flavor, edibility, digestibility, nutritional value, color (which is now recognized as strongly influenced by many phenylpropanoid compounds), scents, and so forth. Recently, phenylpropanoid compounds have been recognized as beneficial nutraceuticals or pharmaconutrients—pharmacologically active compounds that influence (e.g., potentiate, antagonize or otherwise modify) physiological, metabolic, and genetic functions. When selected properly, these nutraceuticals/pharmaconutrients provide health benefits, including but not limited to preventing or reversing ageing or the signs of aging, reducing chronic disease, increasing resistance to acute disease, and so forth. Iriti & Faoro (Current Topics in Nutraceutical Research, 2(1):47-65, 2004) describe representative phenylpropanoids that are found in foods, with particular emphasis to their origin, sources and effects on human health.

Polyphenols are widely distributed in plants, fruits, and vegetables and have received considerable attention because of their physiological functions in human and animal health, including antioxidant, antimutagenic and cancer prevention activities (Salvia et al., J. Agric. Food Chem. 39: 1549-1552, 1991; Bomser et al., Cancer Lett., 135: 151-157, 1999; Zhao et al., Carcinogenesis, 20: 1737-1745, 1999). Epidemiological studies have suggested that flavonoids, among the polyphenols, may reduce the risk of heart disease (Hertog et al., Lancet: 342: 1007-1011, 1993). Additionally, dietary flavan-3-ols and/or proanthocyanidins have been shown to reduce the incidence of atherosclerosis and coronary heart disease in experimental animals (Tijburg et al., Atherosclerosis, 135: 37-47, 1997; Yamakoshi et al., Atherosclerosis, 142: 139-149, 1999). One of the mechanisms responsible for these effects involves their inhibition of oxidation of low density lipoprotein (LDL) (Steinberg, Circulation, 85: 2337-2344, 1992).

Many are polyphenols such as the flavonoids, anthocyanins, and tannins localized mainly in berry skins and seeds (though they are also found in other plants and plant parts). Such pigments are usually antioxidants and thus have oxygen radical absorbance capacity (“ORAC”) that is high among plant foods (Wu et al., J. Agric. Food Chem. 52(12):4026-4037, 2004). Together with good nutrient content, ORAC distinguishes several berries within a new category of functional foods called “superfruits.”

Carotenoids are potent antioxidants, which are believed to quench/interact with certain types of free radicals. This family of compounds includes both carotenes such as β-carotene, and xanthophylls such as lutein, lycopene and astaxanthin. Carotenoids work to remove oxidative agents primarily by quenching singlet oxygen and scavenging free radicals to prevent and terminate chain reactions. Astaxanthin is particularly potent in quenching singlet oxygen, and has over five hundred times the ability to quench singlet oxygen as α-tocopherol. It has a unique molecular structure that gives it powerful antioxidant function. It is extracted from salmon, crustaceans, microalgae, and Phaffia (a yeast, also known as Pfaffia), and it can be chemically synthesized.

The usefulness of secondary metabolites from plants has long been recognized. However, it is only with recent technological advances that we are beginning to be able to exploit specific plant metabolites for specific health benefits. There remains a need for systems, methods, devises, and compositions that can be used to identify, produce, and exploit plant derived products—particularly secondary metabolites such as antioxidants—for influencing health, longevity, aging, and so forth.

### SUMMARY

Provided herein are methods of identifying, characterizing, and using agents that modulate the lifespan, health, etc. of a cell, tissue, organ, or organism (e.g., plant or animal cells, tissues, organs, or organism, as well as microbial organisms). Provided methods involve growing cells (e.g., plant or other cells) in culture in vitro, optionally inducing (e.g., through elicitation with a compound or condition, or set thereof) production of metabolite(s), and harvesting the metabolites from the culture. Various example elicitation substances and conditions are provided, along with methods for varying and optimizing the effects of elicitors on in vitro cell culture. Methods are also provided for characterizing such metabolites with regard to their physical, chemical, biochemical, and biological characteristics—including specifically the ability of the compounds, or extracts from the cells, to influence lifespan, health, longevity etc. of a biological cell, tissue, organ, or organism.

Additional embodiments provide methods of maximizing production of target metabolites, production of formulations comprising extracts or metabolites from the described cell cultures, and methods of using such formulations. The formulations are also provided, including cosmetic, nutritional, and pharmaceutical compositions.

Specific provided methods for identifying an agent that modulates (for instance, extends or shortens) lifespan of a cell, tissue, organ or organism, involve contacting the cell, tissue, organ or organism with a non-animal extract or non-animal-derived composition (e.g., prepared from or derived from plant, fungus, algae, or bacterium cells, for instance genetically modified and/or elicited cells); assessing the influence of the extract or composition on lifespan of the cell, tissue, organ or organism; and selecting the extract or composition as one that modulates lifespan if there is a measurable influence on lifespan of the cell, tissue, organ or organism contacted with the extract or composition in comparison to a corresponding cell, tissue, organ or organism not contacted with the extract or composition, thereby identifying the agent as one that modulates lifespan.

Specifically contemplated sources of extracts are provided herein, though other sources can be identified and the provided lists are not intended to be limiting. Likewise, described herein are specific representative active compounds that are found in some of the extracts and compositions.

In those embodiments in which the source of the extract has been elicited, the elicitation event can comprises one or more of contact with or exposure to: specific wavelength(s) of light; electromagnetic radiation electrical current/potential ionizing radiation high or low light intensity; nitrogen source limitation; carbon source limitation; phosphorus source limitation; water limitation; high salt exposure; high temperature exposure; low temperature exposure; contact stress or wounding; a pathogen-derived compound; a pesticide; a herbicide; a fungicide; a bactericide; anti-viral agent; wounding; a microbial (bacterial, viral, fungal) pathogen or fraction thereof; a nematode or fraction thereof; peroxide; an enzyme; a chemical; a fatty acid; an amino acid; saliva from herbivorous insect or other animal; vibration; gravity or lack thereof, or reduced or increased gravitational field; an extract from a plant; cAMP; ethylene or another gas; and/or a transformation vector (that results in expressing an eliciting compound or protein).

Also provided are a variety of ways to assess the influence of the influence of the extract or composition on lifespan. One such method involves determining if the extract or composition modulates activity or level of at least one telomere length maintenance gene. Other provided methods involve determining if the composition (or extract) modulates the activity or level of one or more specific genes described herein. Also described are methods of assessing the influence of the extract or composition on lifespan by determining if the extract or composition modulates mitochondrial regeneration, biosynthesis, proliferation, maintenance, or function.

Also described herein are methods of modulating (either extending or shortening, depending on the embodiment) the lifespan of a cell, tissue, organ or organism, comprising contacting the cell, tissue, organ or organism with at least one agent identified by one of the methods described herein.

The foregoing and other objects, features, and advantages of the invention will become more apparent from the following detailed description, which proceeds with reference to the accompanying figures.

### BRIEF DESCRIPTION OF THE DRAWINGS

FIG. 1 is a schematic illustration, showing an overview of representative micropropagation systems. Various parts of a plant/flower/fruit can be used to obtain explants which are put into sterile tissue culture, resulting in the development of callus. Under appropriate conditions, the callus can be induced to generate plantlets or embryos that can be grown into plantlets. Any of the cultured tissues can be used as a source of cellular material for the culture production and metabolite generation/harvesting described herein, whether the cultured tissue is fully or partially dedifferentiated, or partially or fully redifferentiated. It is also believed that gene expression and therefore metabolite production can and will be different in each of these conditions, allowing exploitation of different processes and the harvesting of different compounds (or mixtures thereof) depending on the stage from which tissue is used. Likewise, different elicitations will differentially influence gene expression and metabolite production from the different stages of tissue development.

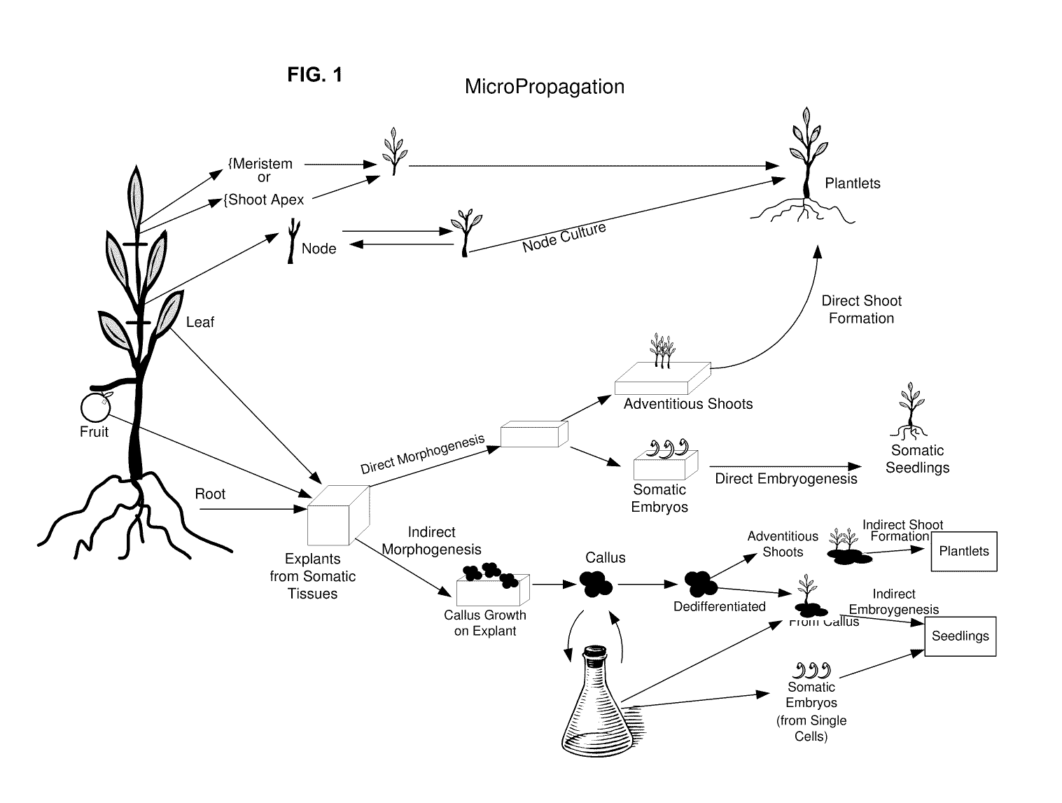

FIG. 2 is a representative bioreactor schematic flow diagram, showing that various environmental influences can be manipulated in order to have (or avoid having) effects on the cultured cells. For instance, temperature, pressure, turbidity, light exposure, viscosity, gas content, pH, and so forth can all be varied (or kept constant). Likewise, the cells used in the bioreactor can be altered—for instance through genetic engineering, chemical modification (e.g., ploidy manipulation through colchicine treatment), and so forth.

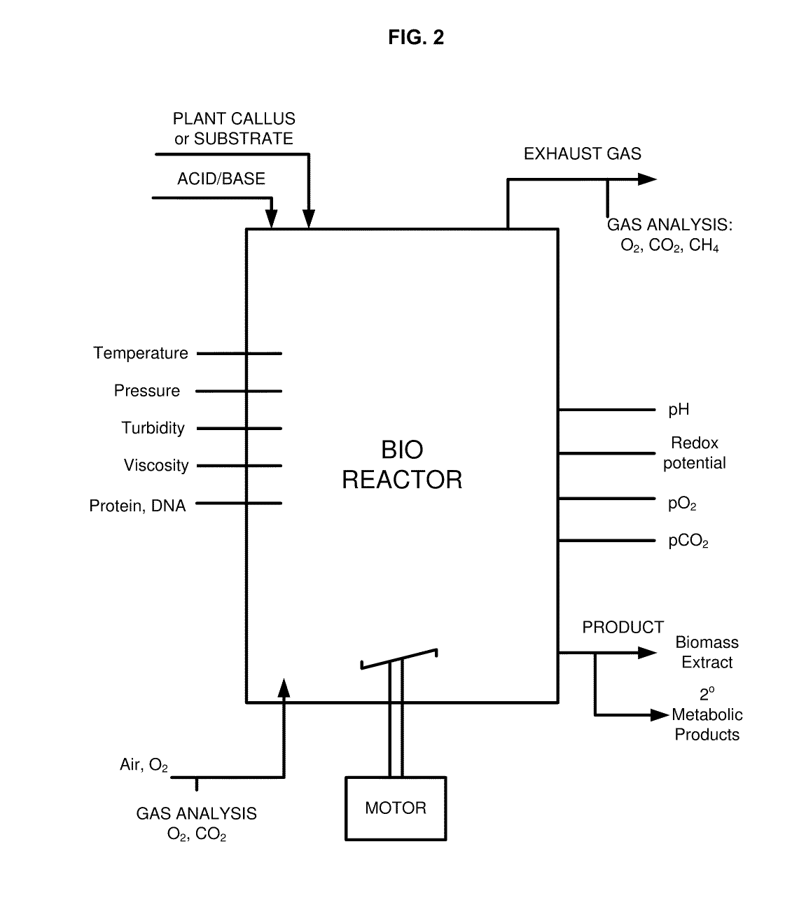

FIG. 3 illustrates basic steps of methods provided herein, whereby plant tissue (for instance, from the leaf, flower, seed, or apical meristem) is cultured to induce callus, optionally subcultured (once or more than once), cultured in suspension medium (with optional elicitation in order to modify production of desired or undesired metabolites), and eventually the metabolic product(s) are harvested from the culture.

FIG. 4 is a representation of a plant, indicating some of the possible elicitation influences that act on plants and modify gene expression and/or other biological responses. Illustrated elicitations include: gas levels (e.g., O₂, N₂, CO₂), temperature (high or low), wounding, pathogen attach and insect attack, light and other radiation exposure, drought, and low (or high) nitrogen, salt, phosphorous, or iron in the soil.

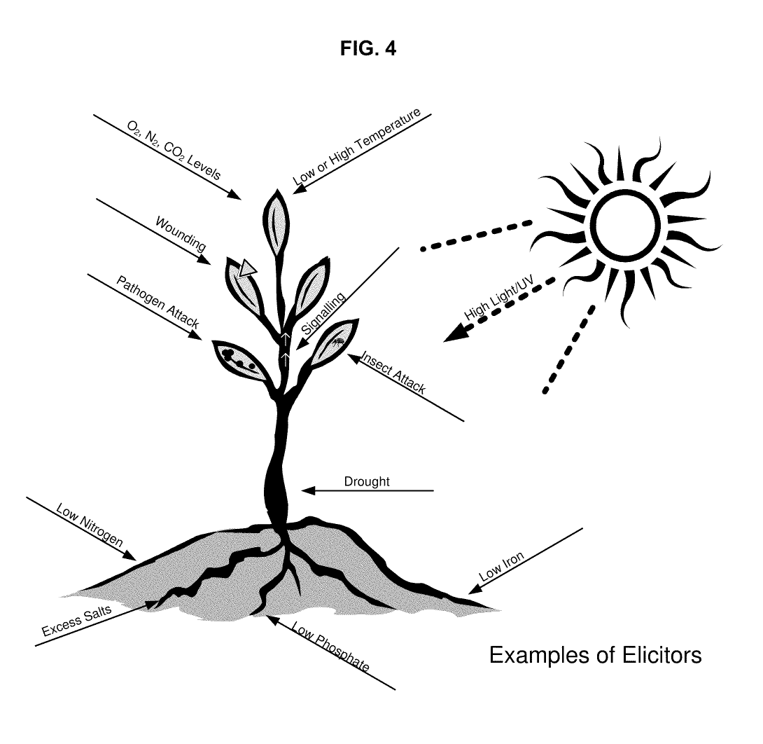

FIG. 5 is a flowchart of the several representative methods for generating the botanical compounds described herein in various embodiments. First, material to be cultured (or that produces at least one desired secondary metabolite) is identified and placed in solid or liquid (shaker) stage I media, depending on which media is expected to provide the better results. The material is placed in a bioreactor with or without specific elicitation conditions, or in the RITA or another similar system used to clone whole plants, or any other method that permits callus tissue/metabolite production. The resultant products are tested in vitro (for instance, through cell culture models, full skin equivalents, etc. . . . with analysis being done by microarray, ELISA and other bioassays) and/or in vivo, and tested in clinical trials, veterinary trials or live plants with multiple metric options (for instance, visual grading, photography, mechanical measurements, blood work, and so forth). It can also be characterized by HPLC mass spectroscopy or other technologies, to identify the compound and its chemical composition and structure, which enables further manipulation of the compound or synthetic production of same.

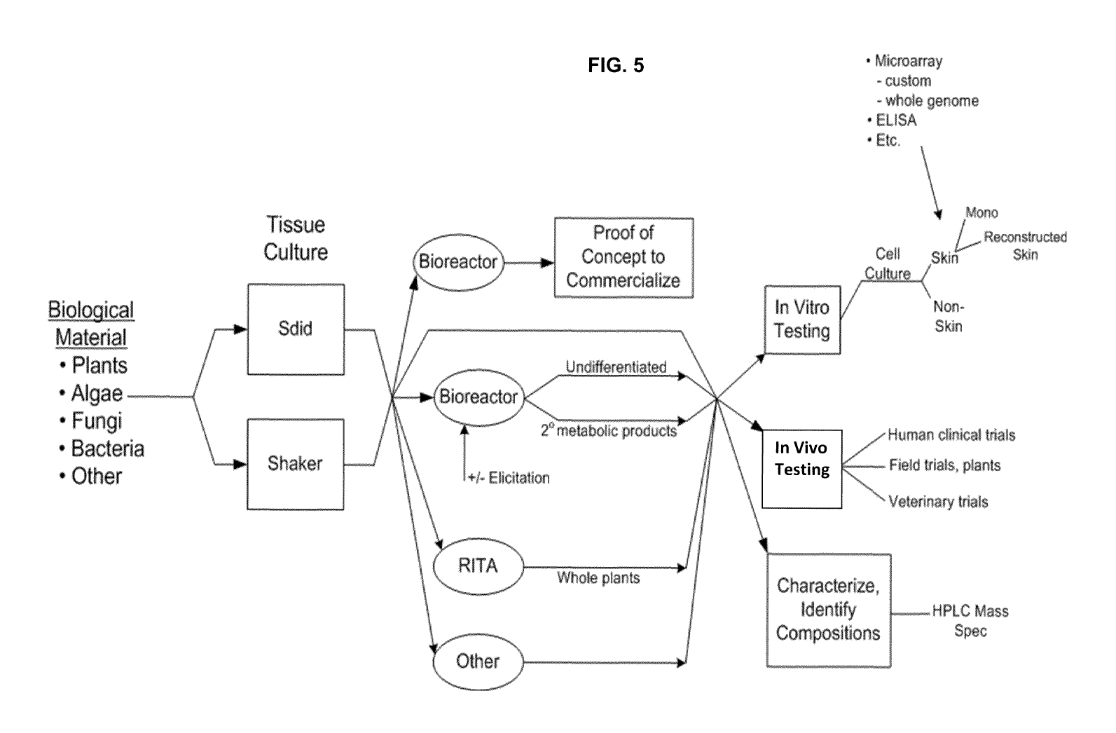

### DETAILED DESCRIPTION

### I. Abbreviations

2,4-D 2,4-dichlorophenoxyacetic acid

2iP 6-(γ,γ-dimethylallylamine)purine

8-OHdG 8-OHdeoxyguanosine

B5 Gamborg's B5

BA Benzyl adenine

CP Chee and Poole

CO₂ carbon dioxide

DNA deoxyribonucleic acid

DKW Driver and Kuniyuki Walnut

ES electrospray

FCW fresh cell weight

FAB/MS Fast atom bombardment/mass spectrometry

GC gas chromatography

HCl hydrochloric acid

HDPE high density polyethylene

HPLC High performance liquid chromatography

H₂O₄ Sulfuric acid

IAA Indole acetic acid

IBA Indole butyric acid

LC Liquid chromatography

LC-MS liquid chromatography-mass spectrometry

LSIMS Liquid secondary ion mass spectrometry

MS Mass spectroscopy

MS Murashige and Skoog (e.g., medium, vitamins, salts)

NAA 1-Naphthalene acetic acid

NMR Nuclear magnetic resonance

NN Nitsch and Nitsch

PCV Packed cell volume

PDA Photodiode array

QL Quiorin and Lepoivre

QSAR quantitative structure activity relationships

RI Refractive index

ROS reactive oxygen species

rpm revolutions per minute

SH Schenk and Hildebrandt

TDZ thidiazuron

TLC thin layer chromatography

VVM volume of gas per volume of culture per minute

WPM McCown's Woody Plant Medium

### II. Terms

Unless otherwise noted, technical terms are used according to conventional usage. In order to facilitate review of the various embodiments of the invention, the following explanations of specific terms are provided:

Addressable: Capable of being reliably and consistently located and identified, as in an addressable location on an array.

Anthocyanins: A group of water-soluble flavonoids that impart pink/red to purple color to leaves and other organs of plants. Common anthocyanins include derivatives of cyanidin, delphinidin, malvidin and pelargonidin. In an example, anthocyanin pigments are pigments formed after cultivation of a Brassica plant callus having reduced glucosinolate in a liquid medium (such as a medium lacking a nitrogen source).

Antioxidant: A substance that, when present in a mixture containing an oxidizable substrate biological molecule, significantly delays, reduces, reverses or prevents oxidation of the substrate biological molecule. Antioxidants can act by scavenging biologically important reactive free radicals or other reactive oxygen species (O_{•-}, H₂O₂, OH⁻, HOCl, ferryl, peroxyl, peroxynitrite, and alkoxyl), or by preventing their formation, or by catalytically converting the free radical or other reactive oxygen species to a less reactive species.

Antioxidant: A molecule or atom capable of slowing or preventing transfer of electrons from one molecule/atom to another (oxidizing agent).

Array: An arrangement of molecules, particularly biological macromolecules (such as polypeptides or nucleic acids) or biological samples (such as tissue sections) in addressable locations on a substrate, usually a flat substrate such as a membrane, plate or slide. The array may be regular (arranged in uniform rows and columns, for instance) or irregular. The number of addressable locations on the array can vary, for example from a few (such as three) to more than 50, 100, 200, 500, 1000, 10,000, or more. A “microarray” is an array that is miniaturized to such an extent that it benefits from microscopic examination for evaluation.

Within an array, each arrayed molecule (e.g., oligonucleotide) or sample (more generally, a “feature” of the array) is addressable, in that its location can be reliably and consistently determined within the at least two dimensions on the array surface. Thus, in ordered arrays the location of each feature is usually assigned to a sample at the time when it is spotted onto or otherwise applied to the array surface, and a key may be provided in order to correlate each location with the appropriate feature.

Often, ordered arrays are arranged in a symmetrical grid pattern, but samples could be arranged in other patterns (e.g., in radially distributed lines, spiral lines, or ordered clusters). Arrays are computer readable, in that a computer can be programmed to correlate a particular address on the array with information (such as identification of the arrayed sample and hybridization or binding data, including for instance signal intensity). In some examples of computer readable array formats, the individual spots on the array surface will be arranged regularly, for instance in a Cartesian grid pattern, that can be correlated to address information by a computer.

The sample application spot (or feature) on an array may assume many different shapes. Thus, though the term “spot” is used herein, it refers generally to a localized deposit of nucleic acid or other biomolecule, and is not limited to a round or substantially round region. For instance, substantially square regions of application can be used with arrays, as can be regions that are substantially rectangular (such as a slot blot-type application), or triangular, oval, irregular, and so forth. The shape of the array substrate itself is also immaterial, though it is usually substantially flat and may be rectangular or square in general shape.

Binding or interaction: An association between two substances or molecules, such as the hybridization of one nucleic acid molecule to another (or itself). Disclosed arrays are used to detect binding of, in some embodiments, a labeled nucleic acid molecule (target) to an immobilized nucleic acid molecule (probe) in one or more features of the array. A labeled target molecule “binds” to a nucleic acid molecule in a spot on an array if, after incubation of the (labeled) target molecule (usually in solution or suspension) with or on the array for a period of time (usually 5 minutes or more, for instance 10 minutes, 20 minutes, 30 minutes, 60 minutes, 90 minutes, 120 minutes or more, for instance over night or even 24 hours), a detectable amount of that molecule associates with a nucleic acid feature of the array to such an extent that it is not removed by being washed with a relatively low stringency buffer (e.g., higher salt (such as 3× SSC or higher), room temperature washes). Washing can be carried out, for instance, at room temperature, but other temperatures (either higher or lower) also can be used. Targets will bind probe nucleic acid molecules within different features on the array to different extents, based at least on sequence homology, and the term “bind” encompasses both relatively weak and relatively strong interactions. Thus, some binding will persist after the array is washed in a more stringent buffer (e.g., lower salt (such as about 0.5 to about 1.5×SSC), 55-65° C. washes).

Where the probe and target molecules are both nucleic acids, binding of the test or reference molecule to a feature on the array can be discussed in terms of the specific complementarity between the probe and the target nucleic acids. Also contemplated herein are protein-based arrays, where the probe molecules are or comprise proteins or peptides, and/or where the target molecules are or comprise proteins or peptides.

Caffeic Acid (3-(3,4-Dihydroxyphenyl 3,4-Dihydroxy-cinnamic acid trans-Caffeate 3,4-Dihydroxy-trans-cinnamate) 2-propenoic acid (E)-3-(3,4-dihydroxyphenyl)-2-propenoic acid 3,4-Dihydroxybenzeneacrylicacid): Formally known as carbolic acid, this phenolic (crystalline acid compound derived from aromatic hydrocarbons) compound can be extracted from the coffee cherry and has been shown to be anti-carcinogenic, anti-inflammatory and have antioxidant properties with a chemical structure similar to cinnamic acid. It is soluble in water and alcohol. Methods for the isolation and characterization of caffeic acid are well known in the art; in addition, this compound is commercially available.

Callus: A mass of undifferentiated cells. A plant cell callus consists of somatic undifferentiated cells from a subject plant, such as an adult subject plant or a plant part including plant embryo. In an example, a callus (such as a red cabbage callus) has reduced glucosinolate content.

Carnosine: A natural amino acid with strong anti-oxidant properties (it helps bind and flush ionic metals from the system). Carnosine has been shown to extend the lifespan of fibroblast cells treated with the amino acid in culture up to 10 divisions past the Hayflick limit of non-treated cells. Carnosine also helps prevent the cross linking of protein and DNA molecules and preventing cell damage.

Catechin 3 gallate (CG): A minor polyphenolic constituent of green tea having antioxidant properties.

Cell Proliferation: The process by which there is an increase in the number of cells as a result of cell growth and division (mitotic cell division).

Cell Senescence: The process of cellular aging and loss of cell function and viability (death).

Chalcone: An aromatic ketone (chemical compound containing a carbonyl C═O group) intermediate in the biosynthesis of flavonoids that forms the central core of many biologically important compounds and has been shown to be able to block voltage dependant potassium channels

Chlorogenic Acid (−[[3-(3,4-Dihydroxyphenyl)-1-oxo-2-propenyfl oxy]-1,4,5-trihydroxycyclo-hexanecarboxylic acid): A family of esters formed between certain trans cinnamic acids and quinic acid (most common individual chlorogenic acid formed from caffeic and quinic acids) and a major phenolic compound found in coffee and the cherry thereof. Chlorogenic acid has been shown to be effective in reducing free radicals (antioxidant ability) and inhibitory to the tumor formation process. Methods for the isolation and characterization of chlorogenic acid are well known in the art; in addition, this compound is commercially available.

Cocoa Bean: A fatty seed from the cacao tree; it contains substantial levels of polyphenols as well as levels of procyanidins. A cacao pod has a rough leathery rind about which varies in thickness dependent on species is filled with sweet, mucilaginous pulp that encases 30 to 50 large beans that are fairly soft and pinkish or purplish in color. It is these beans, containing cocoa butter and cocoa solids (the dried solids produce cocoa powder and the combination of the two creates chocolate in its many incarnations based on the amount of cocoa solids present. Inside the bean and pod itself are the polyphenolic and procyanidin compounds. These compounds have antioxidant anti cancer, nitric oxide (and more generally, free radical) modulatory capabilities, and can have non-steroidal anti-inflammatory effects as well. These polyphenols and procyanidins are commonly extracted from the bean by fermenting, drying and grinding the cocoa seeds.

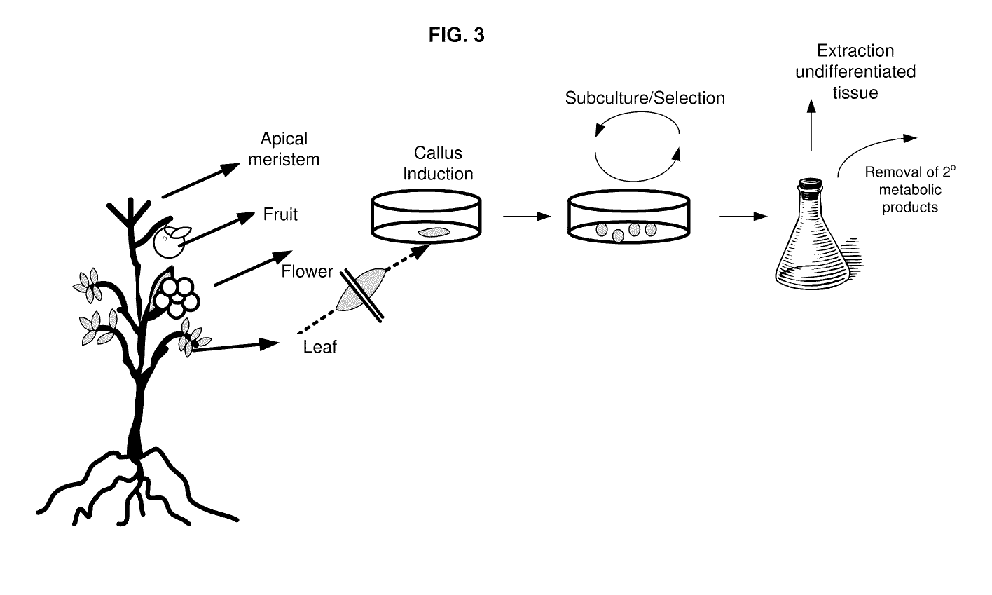

Coffee Cherry: Fruit of the coffee tree Coffea rubiaceae. The pulp, husk (FIG. 3) (to a lesser degree) and mucilage of the whole coffee cherry contain high levels of polyphenols antioxidants if kept in a non fermented state and preserved. The extract of the coffee cherry is generally produced by being contacted with a solvent and will include the nutrients. Further processing of the extract (or “tea”) can allow for the purification of various aspects of the coffee cherry. One commercial producer of a coffee cherry extract is VDF FutureCeuticals, Inc. (Momence, Ill.; marketed as COFFEEBERRY®); a significant portion of their preparation is chlorogenic acid, with the other coffee acids, proanthocyanidins, etc making up the remainder of active ingredients. By way of example, coffee cherry extract can be prepared as described previously (see, e.g., U.S. publication no. 2007/0281048 and other patent documents cited therein; U.S. Publications No. 2006/0210689, 2006/0263508, and 2009/0175973; and PCT publications no. WO 2004/098320, WO 2004/098303, WO 2006/022764 and WO 2004/098320).

Isolation of the coffee acids, including caffeic, chlorogenic, quinic and ferulic acids, as well as proanthocyanidins via (for instance) ion exchange columns and sodium acetate solutions will yield purified antioxidant components. The greatest amounts of antioxidants are found in the green coffee cherries with ripe coffee cherries having somewhat less. Polyphenols constitute a substantial portion of the active ingredients in coffee cherry extract; these polyphenols include chlorogenic acid, caffeine, caffeic acid, ferulic acid, quinic acid, and so forth. Representative analyses of different coffee cherry extracts are shown, for instance, in Table 2 of U.S. Publication Mo. 2007/0281048.

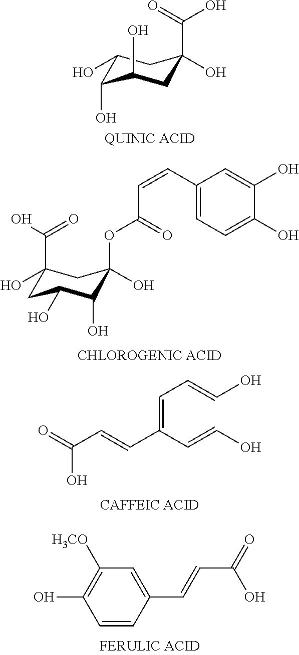

Damage: Any damage resulting from a variety of oxidative agents such as oxygen itself, hydroxyl radical, hydrogen peroxide, other free radicals, ozone etc., or from any kind of harmful irradiation, such as alpha, beta or gamma rays, neutron radiation, and UVA and UVB irradiation.

Dedifferentiated plant cells: Plant cells that lack the features of a particular specialized cell classification, and which are capable of living independently of other cells. Dedifferentiated plant cells can be obtained from plant material that is derived from a whole plant or part of a plant (e.g., leaves, stems, flowers, parts of flowers, anthers, stamens, pistils, petals, roots, fruit, skin, fruit skin, fruit pulp, peel, cuticles, seeds, sap, thorns, buds, peel, and so forth). Dedifferentiated plant cells can be obtained from plants (or parts of plants or ungerminated seeds, etc.) obtained by in vivo culture or derived from in vivo culture. Representative methods that can be employed include those described by E. F. George and P. D. Sherrington in Plantation Propagation by Tissue Culture, Handbook and Directory of Commercial Laboratories (Exegetics Ltd. 1984).

“In vivo culture” means any classical type of culture, i.e. in soil, in the fresh air, in a greenhouse, in a soil-free or hydroponic environment, and so forth. Similarly, the term “in vitro culture” is understood to mean any techniques that enable a plant or a part of a plant to be obtained artificially. The pressure of selection imposed by the physicochemical conditions during the growth of plant cells in vitro enables a standardized plant material to be obtained, which is free from or minimizes (undesirable) contaminants and is available all year round, in contrast to plants cultivated in vivo.

Elicitation: The act of stimulating a change in a cell or organism by contacting it with an elicitor compound or condition. A cell or organism which has undergone an elicitation can be referred to as an elicited cell/organism.

Elicitor: Something that elicits a response. A compound, condition, or environmental stimulus that alters the expression of one or more genes, usually resulting in a change in the physiological or biological characteristics of the cell/organism contacted with the elicitor. Conventional elicitors are compounds (e.g., oligosaccharides) natural plant stress mediators, such as those derived from cellulose fragments released from plant cell that has been damaged, for instance by pathogen invasion, physical damage, etc. Elicitors stimulate the production of a response from a cell or organism, such as production of phytoalexins in response to a pathogen infection. Example elicitors are listed herein, though others will be recognized by those of ordinary skill in the art.

Enantiomer: Enantiomers are forms of a molecule that exist as non-superimposable mirror images of one another. Not being able to superimpose one molecule form on top of the other simply means that the two are not equivalent or identical. For a compound to form an enantiomeric pair, it must have chiral molecules. Chiral molecules must not have an internal plane of symmetry, and they must have a stereocenter. Enantiomers are also called optical isomers because their solutions rotate the plane of polarized light passing through them. If one enantiomer rotates light in the clockwise direction, a solution of the other enantiomer will rotate it in the opposite direction.

Another way to characterize enantiomers is by their configuration. Configuration is the spatial way that non-equivalent groups arrange themselves around a stereocenter carbon. One enantiomer will be configured right handedly (R; rectus) and the other will be configured left handedly (S; sinister). Enantiomers are usually depicted on a planar surface either as a 3-dimensional structural formula or as a Fisher Projection.

Enriched: The term “enriched” means that the concentration of a material is at least about 2, 5, 10, 100, or 1000 times its natural concentration (for example), advantageously at least 0.01% by weight. Enriched preparations of about 0.5%, 1%, 5%, 10%, and 20% by weight are also contemplated.

Enzymatic activity: A detectable (and usually quantifiable) characteristic of at least one function of an enzyme (such as, an OXPHOS enzyme), often monitored over time or in comparison to a standard curve. Methods are well known to those of ordinary skill in the art, for detecting, determining, monitoring, and/or quantifying various enzymatic activities. Also well known are ways of using enzymatic activity assays to assess the ability of compounds (for instance, test compounds) to affect the function of the enzyme, for instance, as an inhibitor or enhancer.

Epigallocatechin gallate (EGCG): The most abundant of the antioxidant catechins found in green tea. It is an ester of epigallocatechol and gallic acid.

Epicatechin gallate (ECG): A polyphenol found in green tea and having antioxidant properties.

Ester: A class of chemical compound that consists of an acid that has at least one —OH (hydroxyl) group replaced by an —O-alkyl (alkoxy) group.

Ferulic Acid ((E)-3-(4-hydroxy-3-methoxy-phenyl)prop-2-enoic acid): A compound serving as a precursor for other aromatic compounds, it is found most commonly in the plant cell walls where it associates with dihydroferulic acid, to facilitate the crosslinking of lignin and polysaccharides conferring rigidity to the cell wall. It can be found in coffee cherry, has antioxidant activity and is biologically synthesized by methylation of caffeic acid. Methods for the isolation and characterization of ferulic acid are well known in the art; in addition, this compound is commercially available.

Free Radicals: Atoms, ions or molecules that contain an unpaired electron. Free radicals are usually unstable, and have short half-lives. Reactive oxygen species (ROS) is a collective term, designating the oxygen radicals (such as the O₂^{•-} superoxide radical), which by sequential univalent reduction produces hydrogen peroxide (H₂O₂) and hydroxyl radical (OH⁻). The hydroxyl radical sets off chain reactions and can interact with nucleic acids. Other ROS include nitric oxide (NO^{•}) and peroxy nitrite (ONO₂^{•}), and other peroxyl (RO₂^{•}) and alkoxyl (RO^{•}) radicals. Increased production of these poisonous metabolites in certain pathological conditions is believed to cause cellular damage through the action of the highly reactive molecules on proteins, lipids and DNA. In particular, ROS are believed to accumulate when tissues are subjected to ischemia, particularly when followed by reperfusion.

Molecular oxygen is essential for aerobic organisms, where it participates in many biochemical reactions, including its role as the terminal electron acceptor in oxidative phosphorylation. Excessive concentrations of various forms of reactive oxygen species and other free radicals can have serious adverse biological consequences, including the peroxidation of membrane lipids, hydroxylation of nucleic acid bases, and the oxidation of sulfhydryl groups and other protein moieties. Biological antioxidants include tocopherols and tocotrieneols, carotenoids, quinones, bilirubin, ascorbic acid, uric acid, and metal binding proteins. These endogenous antioxidant systems are often overwhelmed by pathological processes that allow permanent oxidative damage to occur to tissue.

Gallocatechin gallate (GCG): A member of antioxidant polyphenols found in green tea.

Gnetin H: A stilbene (a hydrocarbon with a trans ethane double bond substituted with a phenyl group on both carbon atoms of the double bond) resveratrol derivative from peony seeds having antioxidant properties and mimicking the effects of resveratrol.

Hayflick Limit: The number of times a cell can undergo mitosis before the telomeres are shortened to a critical length and the cell begins to senesce. Each mitosis event decreases the length of the telomere and pushes the “aging” cell towards senescence. This limit is thought to be a mechanism through which the body can control cancerous cell growth; since the more times a cell undergoes mitosis the more chances for a problematic mutation or transcription error to occur.

Healthy longevity: The concept of having entire organisms (as well as organs, tissues and individual cells) at optimal genetic and functional health. While not limited to these issues, this means for example that the DNA is not significantly damaged or mutated and is in a state comparable to the configuration that would occur in a natural healthy infant or fetus. In other embodiments, the DNA has been altered to be equivalent or better than that status through, e.g., repair or genetic engineering. Similarly, in some embodiments the mitochondrial number and/or function and/or respiratory efficiency are similarly optimal or supra optimal. Metabolic pathways and immune function also may be likewise optimized, and existing environmental damage may have been repaired. Intrinsic chronologic aging and/or oxidative stress damage from normal cellular processes such as free radical damage within mitochondria have also been mitigated or reversed or repaired or otherwise restored to a youthful optimally functional status or a close approximation of the same. Unhealthy cells, including even cancerous cells, which have not been repaired, are eliminated via apoptosis or the death of these cells has been modulated to be accelerated. Significantly gene expression patterns and pathways have been reregulated, or reset or resignalled in such a fashion as to optimize the function and health of the cells and by extension the tissues, organs and organisms that these cells comprise. One end result of at least one or perhaps more of these processes is that the cells achieve maximal longevity or lifespan and/or function optimally or at maximal efficiency and effectiveness for the duration of their lifespan. Understanding that such a process may not be undertaken until substantial damage from aging, disease, diet, injury, environmental exposure, medication or medical therapy side effects, etc. it is understood that even a partial achievement of one or more of these goals would improve the length of the lifespan or make the remaining lifespan duration healthier. Modulating cell function to achieve one or more of these goals is then a means of producing a state termed healthy longevity. The modulation of cell activity to accomplish this may involve in some instances modulating to kill cells prematurely and in a manner diminish the cells health to the point of cell death in order to remove unhealthy cells which may harm the tissue, organ or organism or even which may stimulate the creation and replacement of the unhealthy or sub-optimally healthy cell(s) with new cells via cell division of healthy cells, biogenesis of new cells or replacement of cells via stem cells or autologous transplant or allograft or other types of transplanted cells including genetically engineered cells for transplantation. The treatment of such cells with the process of this invention prior to or after transplantation is also envisioned as a means to produce healthy longevity in these ‘new’ cells.

High throughput genomics: Application of genomic or genetic data or analysis techniques that use arrays, microarrays or other genomic technologies to rapidly identify large numbers of genes or proteins, or distinguish their structure, expression or function from normal or abnormal cells or tissues, or from cells or tissues of subjects with known or unknown phenotype and/or genotype.

Human Cells: Cells obtained from a member of the species Homo sapiens. The cells can be obtained from any source, for example peripheral blood, urine, saliva, tissue biopsy, skin scrape, surgical specimen, amniocentesis samples and autopsy material. From these cells, biological components such as genomic or mitochondrial DNA, mRNA (from which one can make cDNA), RNA, and/or protein can be isolated.

Idebenone (6-(10-hydroxydecyl)-2,3-dimethoxy-5-methyl-1,4-benzoquin-one): Reference German patent document DE3049039, European patent 788793, and U.S. Pat. Nos. 4,436,753, 5,059,627, 5,916,925, application 20050152857 and WIPO 9907355 to describe the use of oral, parenteral or percutaneous preparations of idebenone or derivatives for the treatment of dementia, circulatory disease induction of neural growth factors and resistance to sunburn cell formation. Methods for the isolation and characterization of idebenone are well known in the art; in addition, this compound is broadly commercially available. Idebenone is a synthetic molecule that does not occur in nature and mimics the structure and function of ubiquinone and ubiquinol with similar results for Redox potential and free radical quenching capabilities.

Idebenone has also been shown via chemiluminescence to intercept the pro-oxidative effect of tocopherol oxidation products occurring after 24 hours. In the measurements of the lipid hydroperoxides generated as a result of the oxidation of lipids due to, for example, UV radiation or free radical damage, idebenone was shown to have the highest reduction of said products of the tested antioxidants (U.S. Pat. No. 6,756,045).

Idebenone (chemical) derivative: Derivatives of idebenone may also be suitable for use methods described herein, including the maintenance of telomere length and increase in the longevity of cellular lifespan. Such derivatives may include the salts and/or esters of idebenone, protein bound forms or other derivatives. Examples of idebenone derivatives include esters of idebenone where idebenone is esterified using glycosaminoglycans (GAGS), and/or their salts, for example HA (hyaluronic acid) having a molecular weight of 1 to 1,000,000 and its salts of hyaluronidase inhibitors like inter-alpha-trypsin inhibitor. An example of a hydrophilic idebenone ester is idebenone sulphonic acid.

Injectable Composition: A pharmaceutically acceptable fluid composition comprising at least an active ingredient. The active ingredient is usually dissolved, disseminated, or suspended in a physiologically acceptable carrier, and the composition can additionally comprise minor amounts of one or more non-toxic auxiliary substances, such as emulsifying agents, preservatives, and pH buffering agents and the like. Such injectable compositions that are useful for use with the natural extracts used in methods of this disclosure are conventional; appropriate formulations are well known in the art.

Isolated: An “isolated” biological component (such as a nucleic acid molecule, protein or organelle) has been substantially separated or purified away from other biological components in the cell of the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Lifespan: The length of time a cell, tissue or organism remains viable. There are 2 components to this the Potential (or Inherent) Lifespan defined as the unaltered lifespan of the cell or organism based solely on genetic factors and the Observed Lifespan defined as the length of time the cell or organism will remain viable when all damaging (Oxidative Stress, Poor Nutrition) stimuli are factored in.

Liposome or liposomal: An aqueous compartment or pocket, often microscopic, enclosed by a bimolecular phospholipid membrane; a lipid vesicle. Liposomes have been exploited to deliver compounds and compositions, for instance cells; when the liposome comes in contact with another membrane (e.g., a cell membrane), the two membranes fuse and the encapsulated liposomal contents are released into the cell. This effectively transports the aqueous contents trapped in the liposome across and into the contacted membrane-bound compartment (e.g., cell). Means of preparing liposomes are well known to those of skill in the art. See, e.g., Betageri et al., Liposome Drug Delivery Systems, Technomic Publishing Co., Inc., Lancaster, Pa. (1993).

Meristematic: The quality of being undifferentiated or progenitor cell like, and can apply to both cells and tissues.

A meristem is the tissue in all plants consisting of undifferentiated cells (meristematic cells) and found in zones of the plant where growth can take place. Differentiated plant cells generally cannot divide or produce cells of a different type. Therefore, cell division in the meristem is required to provide new cells for expansion and differentiation of tissues and initiation of new organs, providing the basic structure of the plant body. Meristematic cells are analogous in function to stem cells in animals, are incompletely or not at all differentiated, and are capable of continued cellular division (youthful). Furthermore, the cells are small and protoplasm fills the cell completely. The vacuoles are extremely small. The cytoplasm does not contain differentiated plastids (chloroplasts or chromoplasts), although they are present in rudimentary form (proplastids). Meristematic cells in the plant are packed closely together without intercellular cavities. The cell wall is a very thin primary cell wall.

Metabolite: The intermediates and products of metabolism, such as metabolic intermediates, hormones and other signalling molecules, and secondary metabolites. A primary metabolite is directly involved in normal growth, development, and reproduction. A secondary metabolite is not directly involved in those processes, but usually has an important function (e.g., as an antibiotic or pigment). Secondary metabolites are organic compounds that are not directly involved in the normal growth, development, or reproduction of organisms—and as such, absence of secondary metabolites does not result in immediate death of the cell/organism. Absence of secondary metabolite(s), however, result in long-term impairment for the organism, for instance with regard to survivability, fecundity, or aesthetics. Representative secondary metabolites include mycotoxins and syringomycins (produced by microbes), and caffeine and nicotine (produced by plants); additional classes and categories of secondary metabolites are discussed herein, and/or will be recognized by those of ordinary skill

Mitochondrion (mitochondria): The small, membrane lined organelle providing most of the cells chemical energy through the electron transport systems production of adenosine triphosphate. The mitochondria are also involved in cell growth, cellular signaling, cell cycle regulation, apoptosis, and cellular differentiation. The loss of mitochondrial membrane potentials/functions and deletions of the mtDNA are also thought to be key events in the aging of cells.

Mitochondrial Biogenesis: The process by which new mitochondria are formed during the lifespan of the cell.

Mitochondrial Damage: any physical alteration in mitochondrial components, including mtDNA, proteins (such as, one or more OXPHOS proteins), or lipids, that alters mitochondrial function in a way that is detrimental to cell physiology, growth or faithful replication.

Mitochondrial Disorder: A disease resulting from altered mitochondrial function, caused by any alteration or combination of alterations of mitochondrial components (for instance, mitochondrial protein (such as, one or more OXPHOS proteins), mtDNA, or lipid) caused by genetic and/or environmental factors, including autotoxicity caused by normal cellular metabolic processes. “Late onset mitochondrial disorder” or “late onset disease” means such diseases as late onset diabetes (Diabetes Type I), Huntington's, Parkinson's and Alzheimer's diseases, ALS (amyotrophic lateral sclerosis), Schizophrenia and the like, wherein the subject is free of the disease in early life, but develops the disease during puberty or thereafter, sometimes as late as age 70 or 80.

Nitrogen source: A compound that provides nitrogen to a plant or plant cell culture (such as a callus or suspension culture). Nitrogen sources include ammonium (such as ammonium nitrate or ammonium sulfate) and nitrate (such as ammonium nitrate, potassium nitrate, or calcium nitrate). A growth medium that lacks a nitrogen source is a medium that does not include added nitrogen, such as added nitrogen in the form of ammonium or nitrate.

Nucleic acid array: An arrangement of nucleic acids (such as DNA or RNA) in assigned locations on a matrix, such as that found in cDNA arrays, or oligonucleotide arrays.

Oxygen Radical Absorbance Capacity (ORAC): A method of measuring antioxidant capacities in biological samples (Cao et al., Free Radic Biol Med 14 (3): 303-311, 1993; Ou et al., J Agric Food Chem 49 (10): 4619-4626, 2001). A wide variety of foods have been tested using this methodology, with certain spices, berries and legumes rated very highly (Oxygen Radical Absorbance Capacity of Selected Foods—2007; Nutrient Data Laboratory, Agricultural Research Service, United States Department of Agriculture, November 2007). Correlation between the high antioxidant capacity of fruits and vegetables, and the positive impact of diets high in fruits and vegetables, is believed to play an important role in the free-radical theory of aging.

Oxidative Stress: An imbalance within the cell, tissue or organism which results in a diminished ability to: reduce (or detoxify) biological reactive chemical intermediates, repair the damage caused by reactive chemical intermediates, or maintain the cellular reduction potential most often resulting in apoptosis.

pH: A measure of the acidity or alkalinity of a solution. An aqueous solution at 25° C. with a pH less than seven is acidic, while a solution with a pH greater than seven is considered basic (alkaline) In an example, an acidic pH is less than five, such as between one and three.

Pharmaceutical agent or drug: A chemical compound or composition capable of inducing a desired therapeutic or prophylactic effect when properly administered to a subject.

Phytoalexins: Phytoalexins are relatively low molecular weight metabolite compounds synthesized by plants in response to certain stimuli, including chemical, physical, and biological interactions or insults. Many phytoalexins have antimicrobial activities, and are used by plants to respond to pathogen infections. These compounds are a group of chemically diverse broad, spectrum inhibitors—different plants produce different specific phytoalexins compounds. Phytoalexins tend to fall into several classes including terpenoids, glycosteroids and alkaloids; however, the art recognizes that the term phytoalexin is often to refer to any phytochemicals that are part of the plant's arsenal of defense molecules.

Phytoalexins act as toxins to the attacking organism. Members of this class of compounds puncture the cell wall of some attacking organisms, delay maturation of some, disrupt metabolism or prevent reproduction of a pathogen (or class of pathogens). When a plant cell recognizes particles from damaged cells or particles from the invading organism, the plant activates two resistance pathways—a general short-term response and a delayed long-term specific response. As part of the induced short-term resistance response, the plant deploys Reactive Oxygen Species (ROS) such as superoxide and hydrogen peroxide to kill invading cells. In pathogen interactions, this common short-term response is referred to as the hypersensitivity response, in which cells surrounding the site of infection are signaled to undergo apoptosis, thereby inhibiting spread of the pathogen to the rest of the plant. Long-term resistance, or systemic acquired resistance (SAR), involves communication of the damaged tissue with other parts the plant. These messages are transmitted using plant hormones such as jasmonic acid, ethylene, abscisic acid or salicylic acid. The reception of the signal leads to global gene expression and downstream physiological changes within the plant, which induce genes that protect from further pathogen intrusion, including enzymes involved in the production of phytoalexins.

Plant cell: Any cell derived from a plant, including cells from undifferentiated tissue (e.g., callus) as well as plant seeds, pollen, propagules and embryos. Plant cells can be obtained from any plant organ or tissue and cultures prepared therefrom.

Plant part: Any plant organ or tissue, including, without limitation, seeds, embryos, meristematic regions, callus tissue, leaves, roots, shoots, stems, gametophytes, sporophytes, pollen, and microspores.

Polyphenols (some of which may be referred to as Tea Derived Antioxidants): Ester bond containing polyphenols like EGCG (epigallocatechin-3-gallate), EGC (epigallocatechin), ECG (epicatechin-3-gallate), EC (epicatechin), GCG (gallocatechin gallate), GC (gallocatechin), C (catechin) and/or CG (catechin gallate) can be used to extend the lifespan of living cells through direct influence over the gene expression of the telomere length maintenance unit and related proteins. This extension or preservation of the length of the telomere will increase the replicative capacity and time until apoptosis in living cells resulting in a prolonged duration of cell “health ” and viability. Methods for the isolation and characterization of polyphenols are well known in the art; in addition, various purified polyphenols are commercially available.

Proanthocyanidins (Oligomeric Proanthocyanidin; OPC): A class of flavonoids (plant secondary metabolic products including catechins) most commonly found in many plants, with the extracting into wine being the most common occurrence. They area also found in coffee cherry, and extracts made therefrom, and have been shown to be able to absorb many oxygen free radicals. Methods for the isolation and characterization of proanthocyanidins are well known in the art; in addition, specific proanthocyanidin compounds are commercially available.

Procyanidins: Tannic (polyphenols compounds that bind or shrink proteins) compounds found in plants and especially tea and grape seed. Procyanidins are commonly associated with the bitter, astringent taste of wine. The compounds also have a very high antioxidant capacity. Methods for the isolation and characterization of procyanidins are well known in the art; in addition, certain procyanidins are commercially available.

Purified: The term purified does not require absolute purity; rather, it is intended as a relative term. Thus, for example, a purified nucleic acid preparation is one in which the specified protein is more enriched than the nucleic acid is in its generative environment, for instance within a cell or in a biochemical reaction chamber. A preparation of substantially pure nucleic acid may be purified such that the desired nucleic acid represents at least 50% of the total nucleic acid content of the preparation. In certain embodiments, a substantially pure nucleic acid will represent at least 60%, at least 70%, at least 80%, at least 85%, at least 90%, or at least 95% or more of the total nucleic acid content of the preparation.

Quinic Acid (1S,3R,4S,5R)-1,3,4,5-tetrahydroxy-cyclohexanecarboxylic acid: Discovered in the 1800s, this crystalline acid compound is formed synthetically by hydrolysis of chlorogenic acid, but is found naturally in the coffee cherry. Thought to provide the “acidity” of coffee, this compound, aside from the usual antioxidant capabilities of the other coffee cherry acids, is a versatile starting compound for the synthesis of new synthetic compounds as well. Methods for the isolation and characterization of quinic acid are well known in the art; in addition, this compound is commercially available.

Reactive Oxygen Species (ROS): Very small, organic or inorganic, highly reactive ions or molecules having unpaired electrons in a valence shell including but not limited to free radicals, peroxides and oxygen ions.

Resveratrol (trans 3,4′,5-trihydroxystilbene) belongs to a family of compounds known as phytoalexins. These compounds are synthesized by various plants including grapes, knotweed, blueberries, some pine trees and other plants as part of their natural defense mechanisms in response to stress, injury, invasion by fungi or UV damage. In grapes they are concentrated in the grape skin where they protect from UV damage and function as anti bacterial and anti viral agents. Resveratrol activates the sirtuins which are enzymes which produce at least part of the effects of caloric restriction in living organisms and caloric restriction has been shown in a very wide range of species tested to extend the lifespan of those organisms.

The activation of a sirtuin deacetylase protein family member may be used to produce lifespan extension by mimicking caloric restriction in contrast to extending lifespan by protecting or repairing telomeric structure in cells. Activating compounds may be polyphenol(s) from plants such as chalcone, stilbene, flavone or other sirtuin modulating compounds derived from plants or created by other synthetic processes described herein. Methods for the isolation and characterization of resveratrol are well known in the art; in addition, this compound is commercially available.

Separate(d)/Separation: To spatially dissociate components, such as biomolecules. The components (for example, proteins or peptides) are usually separated based on one or more specific characteristics, such as molecular weight or mass, charge or isoelectric point, conformation, association in a complex, and so forth. Separation may be accomplished by any number of techniques, such as sucrose gradient centrifugation, aqueous or organic partitioning (e.g., 2-phase partitioning), non-denaturing gel electrophoresis, isoelectric focusing gel electrophoresis, capillary electrophoresis, isotachyphoresis, mass spectroscopy, chromatography (e.g., HPLC), polyacrylamide gel electrophoresis (PAGE, such as SDS-PAGE), and so forth.

Once a sample is subjected to a separation, it can be divided into sub-samples or fractions. These fractions may be divided in an order, which may be correlated for instance with a characteristic that was used to separate the components. Thus, a sample subjected to sucrose gradient separation can logically be divided into fractions based on the final density. Proteins or other biomolecules that are separated by an isoelectric focusing gel can be fractionated (e.g., the gel divided into strips) that are correlated with their net charge. Likewise, molecules subjected to SDS-PAGE separation can be fractionated based on their molecular weight. The division of a separated sample into fractions, in some order based on that separation, is well known to those of ordinary skill in the art.

As used herein, separation is not an absolute term (in that separation need not be perfect or “complete” for components to be “separated”). Thus, when a sample is subjected to a separation technique and the resultant separated sample is divided into fractions (e.g., fractions from a sucrose gradient, bands from a gel, and so forth), components within the sample can still be referred to as “separated” even though they occur in more than one of the fractions.

Solid support (or substrate): Any material which is insoluble, or can be made insoluble by a subsequent reaction. Numerous and varied solid supports are known to those in the art and include, without limitation, nitrocellulose, the walls of wells of a reaction tray, test tubes, polystyrene beads, magnetic beads, membranes, microparticles (such as latex particles), and sheep (or other animal) red blood cells. Any suitable porous material with sufficient porosity to allow access by detector reagents and a suitable surface affinity to immobilize capture reagents (e.g., monoclonal antibodies) is contemplated by this term. For example, the porous structure of nitrocellulose has excellent absorption and adsorption qualities for a wide variety of reagents, for instance, capture reagents. Nylon possesses similar characteristics and is also suitable. Microporous structures are useful, as are materials with gel structure in the hydrated state.

Further examples of useful solid supports include: natural polymeric carbohydrates and their synthetically modified, cross-linked or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers which may be prepared with suitably porous structures, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; porous inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer.

It is contemplated that porous solid supports, such as nitrocellulose, described hereinabove are preferably in the form of sheets or strips. The thickness of such sheets or strips may vary within wide limits, for example, from about 0.01 to 0.5 mm, from about 0.02 to 0.45 mm, from about 0.05 to 0.3 mm, from about 0.075 to 0.25 mm, from about 0.1 to 0.2 mm, or from about 0.11 to 0.15 mm The pore size of such sheets or strips may similarly vary within wide limits, for example from about 0.025 to 15 microns, or more specifically from about 0.1 to 3 microns; however, pore size is not intended to be a limiting factor in selection of the solid support. The flow rate of a solid support, where applicable, can also vary within wide limits, for example from about 12.5 to 90 sec/cm (i.e., 50 to 300 sec/4 cm), about 22.5 to 62.5 sec/cm (i.e., 90 to 250 sec/4 cm), about 25 to 62.5 sec/cm (i.e., 100 to 250 sec/4 cm), about 37.5 to 62.5 sec/cm (i.e., 150 to 250 sec/4 cm), or about 50 to 62.5 sec/cm (i.e., 200 to 250 sec/4 cm). In specific embodiments of devices described herein, the flow rate is about 62.5 sec/cm (i.e., 250 sec/4 cm). In other specific embodiments of devices described herein, the flow rate is about 37.5 sec/cm (i.e., 150 sec/4 cm).

The surface of a solid support may be activated by chemical processes that cause covalent linkage of an agent (e.g., a capture reagent) to the support. However, any other suitable method may be used for immobilizing an agent (e.g., a capture reagent) to a solid support including, without limitation, ionic interactions, hydrophobic interactions, covalent interactions and the like. The particular forces that result in immobilization of an agent on a solid phase are not important for the methods and devices described herein.

A solid phase can be chosen for its intrinsic ability to attract and immobilize an agent, such as a capture reagent. Alternatively, the solid phase can possess a factor that has the ability to attract and immobilize an agent, such as a capture reagent. The factor can include a charged substance that is oppositely charged with respect to, for example, the capture reagent itself or to a charged substance conjugated to the capture reagent. In another embodiment, a specific binding member may be immobilized upon the solid phase to immobilize its binding partner (e.g., a capture reagent). In this example, therefore, the specific binding member enables the indirect binding of the capture reagent to a solid phase material.

Except as otherwise physically constrained, a solid support may be used in any suitable shapes, such as films, sheets, strips, or plates, or it may be coated onto or bonded or laminated to appropriate inert carriers, such as paper, glass, plastic films, or fabrics.

Stressed Cells: Cells not able to function fully in their expected capacity either through chemical, biological, or mechanical interference by an outside agent including but not limited to: free radicals, ROS, toxins, UV radiation and genetic inhibitors like siRNAs.

Suffruticosol A and B: Stilbenes (a hydrocarbon with a trans ethane double bond substituted with a phenyl group on both carbon atoms of the double bond), resveratrol derivatives from peony seeds having antioxidant properties and mimicking the effects of resveratrol.

Suspension culture: The growth of cells separate from the organism in which they normally arise. This is typically facilitated via use of a liquid medium. Suspension culture refers to the growth of cells in liquid nutrient media.

Supercritical Fluid Extraction (SFE or SCFE): Supercritical fluids are highly compressed gases that combine properties of gases and liquids. Supercritical fluids (e.g., supercritical fluid carbon dioxide) can be used to extract compounds, such as lipophilic or volatile compounds, from samples. Supercritical fluids are inexpensive, contaminant free, less costly to dispose of safely than organic solvents, and have solvating powers similar to organic solvents, but with higher diffusivities, lower viscosity, and lower surface tension. The solvating power can be adjusted by changing the pressure or temperature of the extraction process, or by adding modifiers to the supercritical fluid.

A typical supercritical fluid extractor consists of a tank of the mobile phase, such CO₂, a pump to pressurize the gas, an oven containing the extraction vessel, a restrictor to maintain a high pressure in the extraction line, and a trapping vessel. Analytes are trapped by letting the solute-containing supercritical fluid decompress into an empty vessel, through a solvent, or onto a solid sorbent material.

Examples of extraction systems are dynamic, static, or combination modes. In a dynamic extraction system, the supercritical fluid continuously flows through the sample in the extraction vessel and out the restrictor to the trapping vessel. In static system, the supercritical fluid circulates in a loop containing the extraction vessel for some period of time before being released through the restrictor to the trapping vessel. In a combination system, a static extraction is performed for some period of time, followed by a dynamic extraction.

The use of supercritical fluid extraction to obtain natural compounds and complexes is well known in the art. See, for instance, Natural Extracts Using Supercritical Carbon Dioxide, by Mamata Mukhopadhyay (CRC Press LLC, Boca Raton, Fla., 2000, ISBN 0-8493-0819-4).

Therapeutically effective dose or amount: A quantity of a substance, such as an antioxidant, sufficient to achieve a desired effect in a subject being treated. The effective amount of a specific substance will be dependent on the subject being treated, the severity of the affliction, and the manner of administration of the substance.

The therapeutically effective amount of a substance, such as the therapeutically effective amount of an antioxidant, can be determined by various methods, including generating an empirical dose-response curve, predicting potency and efficacy of a congener by using quantitative structure activity relationships (QSAR) methods or molecular modeling, and other methods used in the pharmaceutical sciences. Since oxidative damage is generally cumulative, there is no minimum threshold level (or dose) with respect to efficacy. However, minimum doses for producing a detectable therapeutic or prophylactic effect for particular conditions can be established.

Tissue culture: Tissue culture commonly refers to the culture of cells and tissues on solid nutrient media.

Ubiquinone (also known as Coenzyme Q10): A key component of the electron transport/cellular respiration/energy production mechanism, ubiquinone is found in the mitochondria of most eukaryotic cells and in great abundance in cells that have high energy requirements (heart, liver, etc.). Through the process of aerobic cellular respiration ATP is created for use by the cell (95% of all energy in the human body is created in this fashion). Ubiquinone has an affinity for electron transfer and is intimately involved in mitochondrial cellular respiration specifically between Complex II and III where it acts as a transfer agent. Since ubiquinone is a Redox (oxidative reduction) agent, it demonstrates free radical quenching capabilities. The fully oxidized form of the compound is known as ubiquinone, when absorbed into the body 90% of it converts to the “active” antioxidant form of ubiquinol. Methods for the isolation and characterization of ubiquinone are well known in the art; in addition, this compound is commercially available.

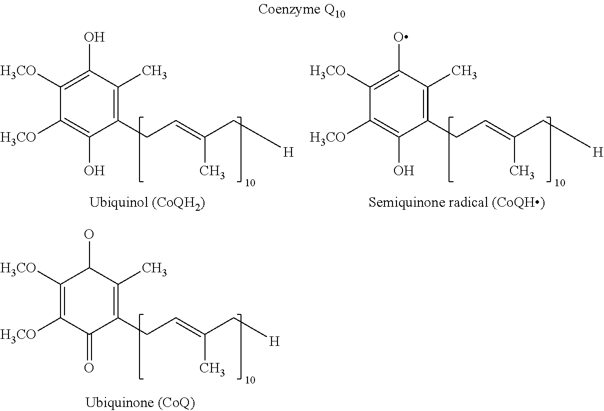

UVA1: A subset of wavelengths in one of the three “bands” of solar lights Ultraviolet Radiation (UVA, UVB and UVC) in the relatively higher power, longer wavelength range of 340 nm-400 nm. UVA2: Solar radiation wavelength range of 320 nm-340 nm. UVB: Solar radiation between the wavelengths of 280 nm-315 nm, capable of causing direct damage to the DNA of cells. UVC: The short, highest energy wavelength radiation (100 nm-280 nm) that is generally filtered by the atmosphere.

Viniferin: A stilbene (a hydrocarbon with a trans ethane double bond substituted with a phenyl group on both carbon atoms of the double bond), resveratrol derivative from peony seeds having antioxidant properties and mimicking the effects of resveratrol.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Overview of Several Embodiments

Provided in a first embodiment is a method for identifying an agent that modulates lifespan of a cell, tissue, organ or organism, the method comprising contacting the cell, tissue, organ or organism with a non-animal extract or non-animal-derived composition; assessing the influence of the extract or composition on lifespan of the cell, tissue, organ or organism; and selecting the extract or composition as one that modulates lifespan if there is a measurable influence on lifespan of the cell, tissue, organ or organism contacted with the extract or composition in comparison to a corresponding cell, tissue, organ or organism not contacted with the extract or composition, thereby identifying the agent that modulates lifespan. Extracts or compositions for use in such methods may be prepared from or derived from plant, fungus, algae, or bacterium cells, for instance. Optionally, such plant, fungus, algae, or bacterium cells are genetically modified. Also (and separately) optionally, such plant, fungus, algae, or bacterium cells are subjected to one or more mechanical, chemical, or biological elicitation event(s) prior to preparation of the extract or composition. Also (and separately) optionally, the plant, fungus, or algae cells are grown in tissue culture prior to preparation of the extract or composition.

By way of example the elicitation event in various embodiments comprises one or more of contact with or exposure to: specific wavelength(s) of light; electromagnetic radiation electrical current/potential ionizing radiation high or low light intensity; nitrogen source limitation; carbon source limitation; phosphorus source limitation; water limitation; high salt exposure; high temperature exposure; low temperature exposure; contact stress or wounding; a pathogen-derived compound; a pesticide; a herbicide; a fungicide; a bactericide; anti-viral agent; wounding; a microbial (bacterial, viral, fungal) pathogen or fraction thereof; a nematode or fraction thereof; peroxide; an enzyme; a chemical; a fatty acid; an amino acid; saliva from herbivorous insect or other animal; vibration; gravity or lack thereof, or reduced or increased gravitational field; an extract from a plant; cAMP; ethylene or another gas; and/or a transformation vector (that results in expressing an eliciting compound or protein). Additional elicitation events are described herein.

Representative species and varieties of organisms from which the extract or composition is prepared are described herein. By way of example, in some embodiments, the extract or composition is prepared from or derived from a plant of the family Rubiaceae, a plant of the family Theaceae, a plant of the family Orchidaceae, a plant of the family of Rosaceae, a microalgae, Coffea arabica, Camellia sinensis, Vaccinia species, Vaccinium macrocarpon, Vaccinium mebranaceum, Vaccinium formosum, Vaccinium alaskensis, Euterpe oleracea, Sequoiadendron giganteum, Sequoia sempervirens, Boswellia sacra, Fragaria virginiana, Vitis rotundifolia, Haematococcus pluvialis, a Phaffia yeast species, or another plant or other organism listed herein.

Agents identified using methods provided herein, in some embodiments, extend lifespan. In other embodiments, they shorten lifespan.

In examples of the provided methods for identifying agents, assessing the influence of the extract or composition on lifespan comprises determining if the extract or composition modulates activity or level of at least one telomere length maintenance gene. For instance, the telomerase length maintenance gene in some examples is selected from the group consisting of TERT, TERC, NRF2, POT1, TRF1, TRF2, TIN2, TPP1, RAP1, TNKS, TNKS 2, TERF2, TERF2IP, POLG, POLB, POLD3, POLE, POLI, POLL, PARP2, PPARG, SHC1, PTOP, IF144, NFKB1, HSPA1A, HSPA1B, HSPA1L, MTND5, HPGD, IDH2, MDH1,MDH2, ME1, ME2, ME3, MTHD1, MTHFD1L, MTHFR, NADK, NADSYN1, NDUFA2, NDUFA3, NDUFA4, NDUFA4L2, NDUFA5, NDUFA6, NDUFA7, NDUFA9, NDUFA10, NDUFA12, NDUFB2, NDUFB3, NDUFB5, NDUFB6, NDUFB7, NDUFB8, NDUFB9, NDUFC2, NDUFS2, NDUFS4, NDUFS5, NDUFS7, NDUFS8, NDUFV2, NDUFV3, NOX1, NOX3, NOX4, NOX5, NOXA1, NOXO1, NQO1, FOXO1, FOXO3, FOXO4, LMNA, NHP2L1, RAD50, RAD51, KL and KU70.

In other method for identifying agents embodiments, assessing the influence of the extract or composition on lifespan comprises determining if the composition modulates activity or level of at least one of: (a) the genes listed as part of Array 1; (b) the genes listed as part of Array 2; (c) VEGFA, HMOX1, CCL4L1, DDC, NOS2A, SIRT1, TERT, PTGS2, or IFI44; (d) four or more of TERT, TERC, NRF2, POT1, TRF1, TRF2, TIN2, TPP1, RAP1, TNKS, TNKS 2, TERF2, TERF2IP, POLG, POLB, POLD3, POLE, POLI, POLL, PARP2, PPARG, SHC1, PTOP, IFI44, NFKB1, HSPA1A, HSPA1B, HSPA1L, MTND5, HPGD, IDH2, MDH1, MDH2, ME1, ME2, ME3, MTHD1, MTHFD1L, MTHFR, NADK, NADSYN1, NDUFA2, NDUFA3, NDUFA4, NDUFA4L2, NDUFA5, NDUFA6, NDUFA7, NDUFA9, NDUFA10, NDUFA12, NDUFB2, NDUFB3, NDUFB5, NDUFB6, NDUFB7, NDUFB8, NDUFB9, NDUFC2, NDUFS2, NDUFS4, NDUFS5, NDUFS7, NDUFS8, NDUFV2, NDUFV3, NOX1, NOX3, NOX4, NOX5, NOXA1, NOXO1, NQO1, FOXO1, FOXO3, FOXO4, LMNA, NHP2L1, RAD50, RAD51, KL and KU70; (e) BCL2, SOD1, TP53, and SOD2; (f) BCL2, SOD1, TP53, SOD2, BCL2L1, TIMM22, TOMM40, IMMP1L, CDKN2A, GAPDH, ACTB, HRP1, and HGDC; (g) PARP1, PARP2, TERT, TEP1, TPS3, JUN, PARP3, PARP4, TERF2, TINF2, and CDKN2A; (h) PARP1, PARP2, TERT, TEP1, and TP53; (i) TERF2, POT1, TERT, and TPP1; (j) PAPR1, PARP2, PARP3, and PARP4; (k) PARP2, CYP19A1, TEP1, BCL2, HSPA1A, ACE, TP53, and NFKB1; (l) IGF1, IGF2, PPARG, IL10, APOE, TERT, TNF, HLA-DRA, DDC, CCL4L1, NOS2A, and GH1; (m) PARP1, IL6, SIRTT1, KRAS, and HSPA1L; (n) IGF1, IL6, PPARG, IL10, TERT, TNF, TEP1, HSPA1A, SIRT1, TP53, GH1, NOS2A, and PPC; or (o) a combination of two or more of (a) through (n).

In yet further examples of the methods for identifying agents, assessing the influence of the extract or composition on lifespan comprises determining if the extract or composition modulates mitochondrial regeneration, biosynthesis, proliferation, maintenance, or function. For instance, assessing the influence of the extract or composition on lifespan in some instances comprises determining if the extract or composition modulates activity or level of at least one of: (a) the genes listed as part of Array 1; (b) the genes listed as part of Array 2; (c) VEGFA, HMOX1, CCL4L1, DDC, NOS2A, SIRT1, TERT, PTGS2, or IFI44; (d) four or more of TERT, TERC, NRF2, POT1, TRF1, TRF2, TIN2, TPP1, RAP1, TNKS, TNKS 2, TERF2, TERF2IP, POLG, POLB, POLD3, POLE, POLI, POLL, PARP2, PPARG, SHC1, PTOP, IFI44, NFKB1, HSPA1A, HSPA1B, HSPA1L, MTNDS, HPGD, IDH2, MDH1,MDH2, ME1, ME2, ME3, MTHD1, MTHFD1L, MTHFR, NADK, NADSYN1, NDUFA2, NDUFA3, NDUFA4, NDUFA4L2, NDUFA5, NDUFA6, NDUFA7, NDUFA9, NDUFA10, NDUFA12, NDUFB2, NDUFB3, NDUFB5, NDUFB6, NDUFB7, NDUFB8, NDUFB9, NDUFC2, NDUFS2, NDUFS4, NDUFSS, NDUFS7, NDUFS8, NDUFV2, NDUFV3, NOX1, NOX3, NOX4, NOX5, NOXA1, NOXO1, NQO1, FOXO1, FOXO3, FOXO4, LMNA, NHP2L1, RAD50, RAD51, KL and KU70; (e) BCL2, SOD1, TP53, and SOD2; (f) BCL2, SOD1, TP53, SOD2, BCL2L1, TIMM22, TOMM40, IMMP1L, CDKN2A, GAPDH, ACTB, HRP1, and HGDC; (g) PARP1, PARP2, TERT, TEP1, TPS3, JUN, PARP3, PARP4, TERF2, TINF2, and CDKN2A; (h) PARP1, PARP2, TERT, TEP1, and TP53; (i) TERF2, POT1, TERT, and TPP1; (j) PAPR1, PARP2, PARP3, and PARP4; (k) PARP2, CYP19A1, TEP1, BCL2, HSPA1A, ACE, TP53, and NFKB1; (1) IGF1, IGF2, PPARG, IL10, APOE, TERT, TNF, HLA-DRA, DDC, CCL4L1, NOS2A, and GH1; (m) PARP1, IL6, SIRTT1, KRAS, and HSPA1L; (n) IGF1, IL6, PPARG, IL10, TERT, TNF, TEP1, HSPA1A, SIRT1, TP53, GH1, NOS2A, and PPC; or (o) a combination of two or more of (a) through (n).

In yet another embodiment, assessing the influence of the extract or composition on lifespan comprises determining if the extract or composition modulates oxidative DNA damage.

In any of these methods, the method is optionally carried out using a cell, which cell may optionally be in vitro (or in vivo). Such cells may be, for instance, a mammalian cell or a plant cell. In specific methods, the cell is a stem cell. The cells may be eukaryotic cells or prokaryotic cells.

Extracts or compositions used in methods provided herein comprises at least one active compound selected from the group consisting of idebenone or an analog or derivative thereof, (+) catechin, (−) epicatechin, procyanidin oligomers 2 through 18, procyanidin B-5, procyanidin B-2, procyanidin A-2, procyanidin C-1, chlorogenic acid, quinic acid, ferulic acid, caffeic acid, coffee cherry proanthocyanidins, EGCG (epigallocatechin-3-gallate), EGC (epigallocatechin), ECG (epicatechin-3-gallate), EC (epicatechin), GCG (gallocatechin gallate), GC (gallocatechin), C (catechin), CG (catechin gallate), viniferin, gnetin H, suffruticosol B, astaxanthin, β-carotene, lutein, canthaxanthin, or another compound referenced herein. Optionally, the extracts or compositions comprises at least one active compound other than idebenone or an analog or derivative thereof, (+) catechin, (-) epicatechin, procyanidin oligomers 2 through 18, procyanidin B-5, procyanidin B-2, procyanidin A-2, procyanidin C-1, chlorogenic acid, quinic acid, ferulic acid, caffeic acid, coffee cherry proanthocyanidins, EGCG (epigallocatechin-3-gallate), EGC (epigallocatechin), ECG (epicatechin-3-gallate), EC (epicatechin), GCG (gallocatechin gallate), GC (gallocatechin), C (catechin), CG (catechin gallate), viniferin, gnetin H, suffruticosol B, astaxanthin, β-carotene, lutein, canthaxanthin, or another compound referenced herein.

Also provided in another embodiment is a method of modulating the lifespan of a cell, tissue, organ or organism, comprising contacting the cell, tissue, organ or organism with at least one agent identified by a method described herein. Such agent can be, for instance, dissolved in oil, dispersed in oil, dissolved or dispersed in alcohol, dispersed in an aqueous medium, homogenized in an aqueous medium, encapsulated, processed into dry material, or a combination of two or more thereof. For instance, in specific embodiments the agent is processed into dry material, and the form of the dry material is stabilized beadlets, powder, an encapsulated form, granule, or a combination of two or more thereof. In other embodiments, the agent is formulated as a liquid, a liquid capsule, a solid capsule or a tablet. Optionally, the agent is added to a food or beverage product.

Also provided are such preparations, which contain at least one agent identified or characterized by a method herein described.

Yet another embodiment is a cosmetic preparation comprising at least one active component of an extract or composition identified by a method herein described. Optionally, the cosmetic preparation further comprises at least one additional active component, for instance, a carotenoid, an antioxidant, a vitamin, a second natural extract, a sunscreen agent, retinoic acid, retinol, an alpha or beta hydroxyl acid, or another compound or preparation recognized as providing protection to or improvement of skin, health, and/or longevity. Cosmetic preparations as described herein may be formulated for topical application, though other formulations are also provided.

### IV. Identifying, Producing and Using Plant Metabolites for Modulating Cell Function and Aging

Plants and other living organisms have a variety of complex defense and repair mechanisms that help them to survive despite various chemical, mechanical, biological and environmental insults. These defense and/or repair mechanisms help to prevent premature senescence or aging, help to prolong lifespan and also to prolong healthy lifespan. Supplemental assistance with these defensive and/or reparative processes may be beneficial to the living organism in addition to their native endogenous processes.

Extracts, derivatives, metabolites from plants (or other cells, such as bacterial or fungal cells) may be used to benefit the health of living organisms by exposing a cell, tissue, organ or the entire organism via many well described routes. These metabolites can be extracted from wild or controlled agricultural practices, but the chemical composition varies from culture (such as soil, weather, season, etc) as well as with geographic location, species and cultivar of plant and many other factors. Extracts may also be contaminated with pesticides, herbicides, fungicides, plant pathogens, environmental pollutants or chemicals and a host of other factors. The chemical composition and ratio of chemicals cannot be uniformly controlled and also there are seasonal production issues for example with ripening fruit. Even the stage of ripeness or time of harvest may have a very large impact on the chemical composition of plants or various plant parts.

Thus it is desirable to cultivate plants (or bacteria or fungi) or subcomponents of plants in a very highly controlled environment such as a bioreactor, in order to produce repeatable, reliable compounds and compositions. The ability to control the environment in a bioreactor also facilitates the use of various elicitors to stimulate or control the production and ratios of beneficial chemicals (and optionally to reduce undesired chemicals) produced by the plant, bacterial, or fungal cells. In fact the cultured cells become harnessed chemical factories and have the ability to produce very complex molecular structures.

Undifferentiated or dedifferentiated plant tissue or callus tissue may be generated from any part of the plant—including but not limited to a root, stem, apical meristem, flower, flower part such as pollen or anther, fruit, or subcomponents of these (such as the skin of a fruit or the pulp or the seed), and other parts recognized in the art and/or listed herein.

Furthermore these undifferentiated plant cells can be themselves extracted or even the contents of vacuoles or subcellular fractions as well as secondary metabolic products which can be isolated and utilized.

Elicitors of various types may be used to expose or treat this plant tissue thus eliciting a response from the tissue in the gene expression and production of chemicals that are desirable or beneficial or to alter the ratio of chemicals. For example production of a specific antioxidant compound(s) may be initiated or the production increased after exposure to a plant pathogen, or an environmental challenge such as simulated drought or cold or a chemical present in insect saliva simulating an insect attack. A large but non-limiting variety of methods of elicitation are described herein.

The ability to control both the cell growth as well as the production of chemical substances year round in a reliable production schedule without environmental contaminants such as pesticides is highly desirable for both composition and purity, as well as practical commercial production.

Many of the cause of premature aging are related to inflammation and oxidative stress. Antioxidants have significant benefits for protecting and defending cells, tissues, organs and organisms as well as for repairing damage. Plants can be a rich source of antioxidants and the production of these antioxidants can be modulated by controlling the environment of growth as well as the stage of differentiation of the plant cells and also by manipulating the environmental exposure to various elicitors. The effects of these conditions and elicitations may be optimized for some applications by utilizing dedifferentiated cells. Micropropagation of various plant components and in various stages of maturity or immaturity of development also allows control of the chemicals produced.

Thus, provided herein are methods of culturing plants (for instance, tea, coffee, orchid and blueberry) in vitro to produce dedifferentiated (stem cell-like) callus tissue, for instance from apical meristem tissue, from leaves, flowers, flower parts, fruit, or fruit parts. The callus tissue is grown until such time as a large enough quantity of lysate can be generated for cellular/in vitro testing. Plant cell lysates/extracts thus produced (or components or fractions thereof) are evaluated for biological activity(s). For instance, they are in some embodiments evaluated to determine their ability to reverse or prevent the cellular changes or stresses, or genetic expression changes, caused by treat cells with hydrogen peroxide (H₂O₂), UV radiation, hypoxia, etc., in a cell based system, such as cultured human skin fibroblasts and/or cardiac cells. The cells are exposed to the extract following or concurrent with a stressor (e.g., with H₂O₂, UV radiation and/or hypoxia) and RNA is isolated from the cells. RNA is then analyzed for expression changes, in comparison to control cells (e.g., cells not treated with the compound/extract/lysate. By way of example, the RNA is used to assay a PCR microarray, such as a custom microarray containing the genes involved in cell health, longevity, DNA replication or maintenance, mitochondrial maintenance, etc. By way of example, Array 1 or Array 2, provided herein, can be used. The extracts are also tested for whether and how they affect mitochondrial genesis/number and respiration efficiency.

Cellular extracts or metabolites prepared therefrom which show biological activities in vitro or in cell-based systems are evaluated in whole animal systems. By way of example, the extracts (or metabolites prepared therefrom) are evaluated for safety, formulated for use in whole animal systems, and evaluated in human pilot clinical trials for measurable endpoints to be determined prior to study initiation.

Also provided herein are systems for further influencing the production of metabolites from cultured cells, through elicitation of the cells in culture. Plant explants or suspension cultures will be stressed by modulating one or more variables of the growing environment (for instance, light, oxygen, water, carbon source, nitrogen source, etc. . . . ) to determine if cell chemistry and/or genetic expression is affected, and if that affect can alter the effectiveness of the cell extract, its composition, the amount or mixture of metabolite(s) in the extract/lysate, etc.. The “stressed” extracts are then evaluated as described for the non-stressed extracts.

### V. Sources of Tissue Culture Materials

Provided herein are various methods for making from tissue culture (e.g., plant tissue culture, though fungal and bacterial cultures are also contemplated) compositions for modulating gene expression or protein production or cell signaling which controls the maintenance of the telomere and/or which controls the biogenesis or respiratory activity of mitochondria and/or which control the lifespan, rate of aging, senescence, onset of disease states, or response to stress including apoptosis and cell death for a living cell, tissue, organ or organism.

The selection of the plant phyla, genus, and species are important. The geographic region of origin may impact the production of chemicals as well since plant cultivars as well as species may have evolved and adapted to unique environments and are thus more optimally or perhaps uniquely suited to produce certain beneficial chemicals.

For example blueberries grow over very large geographic regions and the ORAC measurements of antioxidant capacity has been well studied for various cultivars of blueberries. Blueberries from Alaska have significantly higher ORAC values than the hybrid blueberries which are widely cultivated commercially. A similar occurrence is seen with cranberries from wild versus cultivated commercial crop varieties. It may be that the blueberries exposed to the harsh environment in Alaska for example have evolved to produce more protective antioxidants than commercial agricultural cultivars which have been hybridized and selected for traits such as larger size or higher sugar content or heat tolerance or disease resistance. Thus the wild Alaskan blueberries may be more desirable for the production of antioxidants.

Another example is coffee. Coffea arabica is 4N or tetraploid in chromosome count whereas other Coffea species may be 2N or diploid. The unripe coffee fruit or coffee cherry has a green skin whereas it becomes bright red on ripening and the chemical content of unripe or semiripe coffee cherries is different than ripe as is the content of the peel versus the seed or coffee bean. Thus the chemicals which can be elicited may differ not only depending on whether the root or apical meristem or the flower or the fruit or the peel or the bean of the coffee plant is utilized, but also the stage of maturity and in this case whether the peel or the bean is utilized. Furthermore whether differentiated cells or undifferentiated cells are used also impacts the resultant chemicals which may be produced or elicited.

Tissue for tissue culture can also be taken from any part of the plant, including for instance topical meristem or bud, root (including root tip, root hairs, and so forth), stem/trunk (including bark peels, exocarp, endocarp, phloem, xylem, and so forth), leaf (including leaf parts), flower (including parts of flowers, such as anther, petals, stamen, pistil, etc.), pollen, seed (and parts of seeds), fruit (and parts or portions of fruits, such as peel, pulp, seed, etc.), cuticle, and so forth. It is advantageous (though not necessary) to produce tissue culture from different parts of a plant in order to evaluate and compare the chemical/metabolite production profile for each.

It is believed that plants from any plant Division, including Bryophyta, Psilophyta, Lycophyta, Equisetophyta, Filicophyta, Coniferophyta, Ginkgophyta, Cycadophyta, Gnetophyta, and Angiospermophyta, can be used in various embodiments provided herein.

Without intending to be limited to particular plants or specific compounds or compositions derived therefrom, the following specific plants are contemplated for preparing cell cultures that produce lifespan influencing compositions: coffee (e.g., coffee cherry extract), green tea (e.g., green tea extract), blueberries (Alaskan, for instance), cranberries, huckleberries, acai berries, goji berries, blackberries, raspberries, grapes (scupernog), strawberries, persimmon, pomegranate, lingonberry, bearberry, mulberry, bilberry, choke cherry, sea buckthorn berries, goji berry, tart cherry, kiwi, plum, apricot, apple, banana, berry, blackberry, blueberry, cherry, cranberry, currant, greengage, grape, grapefruit, gooseberry, lemon, mandarin, melon, orange, pear, peach, pineapple, plum, raspberry, strawberry, sweet cherry, watermelon, and wild strawberry. In addition, extracts produced from cell cultures derived from trees and bushes are also contemplated, including for instance cell cultures from sequoia, coastal redwood, bristlecone pine, birch, cedar of Lebanon, frankincense, and so forth.

By way of additional examples, cell cultures from which compositions or metabolites can be harvested include cell cultures from leafy or salad vegetables [e.g., Amaranth (Amaranthus cruentus), Arugula (Eruca sativa), Beet greens (Beta vulgaris subsp. vulgaris), Bitterleaf (Vernonia calvoana), Bok choy (Brassica rapa Chinensis group), Broccoli Rabe (Brassica rapa subsp. rapa), Brussels sprout (Brassica oleracea Gemmifera group), Cabbage (Brassica oleracea Capitata group), Catsear (Hypochaeris radicata), Celery (Apium graveolens), Celtuce (Lactuca sativa var. asparagina), Ceylon spinach (Basella alba), Chard (Beta vulgaris var. cicla), Chaya (Cnidoscolus aconitifolius subsp. aconitifolius), Chickweed (Stellaria), Chicory (Cichorium intybus), Chinese cabbage (Brassica rapa Pekinensis group), Chinese Mallow (Malva verticillata), Chrysanthemum leaves (Chrysanthemum coronarium), Collard greens (Brassica oleracea), Corn salad (Valerianella locusta), Cress (Lepidium sativum), Dandelion (Taraxacum officinale), Endive (Cichorium endivia), Epazote (Chenopodium ambrosioides), Fat hen (Chenopodium album), Fiddlehead (Pteridium aquilinum, Athyrium esculentum), Fluted pumpkin (Telfairia occidentalis), Garden Rocket (Eruca sativa), Golden samphire (Inula crithmoides), Good King Henry (Chenopodium bonus-henricus), Greater Plantain (Plantago major), Kai-lan (Brassica rapa Alboglabra group), Kale (Brassica oleracea Acephala group), Komatsuna (Brassica rapa Pervidis or Komatsuna group), Kuka (Adansonia spp.), Lagos bologi (Talinum fruticosum), Land cress (Barbarea verna), Lettuce (Lactuca sativa), Lizard's tail (Houttuynia cordata), Melokhia (Corchorus olitorius, Corchorus capsularis), Mizuna greens (Brassica rapa Nipposinica group), Mustard (Sinapis alba), New Zealand Spinach (Tetragonia tetragonioides), Orache (Atriplex hortensis), Paracress (Acmella oleracea), Pea sprouts/leaves (Pisum sativum), Polk (Phytolacca americana), Radicchio (Cichorium intybus), Samphire (Crithmum maritimum), Sea beet (Beta vulgaris subsp. maritima), Seakale (Crambe maritima), Sierra Leone bologi (Crassocephalum spp.), Soko (Celosia argentea), Sorrel (Rumex acetosa), Spinach (Spinacia oleracea), Summer purslane (Portulaca oleracea), Swiss chard (Beta vulgaris subsp. cicla var. flavescens), Tatsoi (Brassica rapa Rosularis group), Turnip greens (Brassica rapa Rapifera group), Watercress (Nasturtium officinale), Water spinach (Ipomoea aquatica), Winter purslane (Claytonia perfoliata), Yarrow (Achillea millefolium)]; fruiting and flowering vegetables, such as those from trees [e.g., Avocado (Persea americana), Breadfruit (Artocarpus altilis)]; or from annual or perennial plants [e.g., Acorn squash (Cucurbita pepo), Armenian cucumber (Cucumis melo Flexuosus group), Aubergine (Solanum melongena), Bell pepper (Capsicum annuum), Bitter melon (Momordica charantia), Caigua (Cyclanthera pedata), Cape Gooseberry (Physalis peruviana), Capsicum (Capsicum annuum), Cayenne pepper (Capsicum frutescen), Chayote (Sechium edule), Chili pepper (Capsicum annuum Longum group), Courgette (Cucurbita pepo), Cucumber (Cucumis sativus), Eggplant (Solanum melongena), Luffa (Luffa acutangula, Luffa aegyptiaca), Malabar gourd (Cucurbita ficifolia), Parwal (Trichosanthes dioica), Pattypan squash (Cucurbita pepo), Perennial cucumber (Coccinia grandis), Pumpkin (Cucurbita maxima, Cucurbita pepo), Snake gourd (Trichosanthes cucumerina), Squash aka marrow (Cucurbita pepo), Sweet corn aka corn; aka maize (Zea mays), Sweet pepper (Capsicum annuum Grossum group), Tinda (Praecitrullus fistulosus), Tomatillo (Physalis philadelphica), Tomato (Lycopersicon esculentum var), Winter melon (Benincasa hispida), West Indian gherkin (Cucumis anguria), Zucchini (Cucurbita pepo)]; the flower buds of perennial or annual plants [e.g., Artichoke (Cynara cardunculus, C. scolymus), Broccoli (Brassica oleracea), Cauliflower (Brassica oleracea), Squash blossoms (Cucurbita spp.); podded vegetables [e.g., American groundnut (Apios americana), Azuki bean (Vigna angularis), Black-eyed pea (Vigna unguiculata subsp. unguiculata), Chickpea (Cicer arietinum), Common bean (Phaseolus vulgaris), Drumstick (Moringa oleifera), Dolichos bean (Lablab purpureus), Fava bean (Vicia faba), Green bean (Phaseolus vulgaris), Guar (Cyamopsis tetragonoloba), Horse gram (Macrotyloma uniflorum), Indian pea (Lathyrus sativus), Lentil (Lens culinaris), Lima Bean (Phaseolus lunatus), Moth bean (Vigna acontifolia), Mung bean (Vigna radiata), Okra (Abelmoschus esculentus), Pea (Pisum sativum), Peanut (Arachis hypogaea), Pigeon pea (Cajanus cajan), Ricebean (Vigna umbellata), Runner bean (Phaseolus coccineus), Soybean (Glycine max), Tarwi (tarhui, chocho; Lupinus mutabilis), Tepary bean (Phaseolus acutifolius), Urad bean (Vigna mungo), Velvet bean (Mucuna pruriens), Winged bean (Psophocarpus tetragonolobus), Yardlong bean (Vigna unguiculata subsp. sesquipedalis)]; bulb and stem vegetables [e.g., Asparagus (Asparagus officinalis), Cardoon (Cynara cardunculus), Celeriac (Apium graveolens var. rapaceum), Celery (Apium graveolens), Elephant Garlic (Allium ampeloprasum var. ampeloprasum), Florence fennel (Foeniculum vulgare var. dulce), Garlic (Allium sativum), Kohlrabi (Brassica oleracea Gongylodes group), Kurrat (Allium ampeloprasum var. kurrat), Leek (Allium porrum), Lotus root (Nelumbo nucifera), Nopal (Opuntia ficus-indica), Onion (Allium cepa), Prussian asparagus (Ornithogalum pyrenaicum), Shallot (Allium cepa Aggregatum group), Welsh onion (Allium fistulosum), Wild leek (Allium tricoccum)]; root and tuberous vegetables [e.g., Ahipa (Pachyrhizus ahipa), Arracacha (Arracacia xanthorrhiza), Bamboo shoot (Bambusa vulgaris and Phyllostachys edulis), Beetroot (Beta vulgaris subsp. vulgaris), Black cumin (Bunium persicum), Burdock (Arctium lappa), Broadleaf arrowhead (Sagittaria latifolia), Camas (Camassia), Canna (Canna spp.), Carrot (Daucus carota), Cassava (Manihot esculenta), Chinese artichoke (Stachys affinis), Daikon (Raphanus sativus Longipinnatus group), Earthnut pea (Lathyrus tuberosus), Elephant Foot yam (Amorphophallus paeoniifolius), Ensete (Ensete ventricosum), Ginger (Zingiber officinale), Gobo (Arctium lappa), Hamburg parsley (Petroselinum crispum var. tuberosum), Jerusalem artichoke (Helianthus tuberosus), Jícama (Pachyrhizus erosus), Parsnip (Pastinaca sativa), Pignut (Conopodium majus), Plectranthus (Plectranthus spp.), Potato (Solanum tuberosum), Prairie turnip (Psoralea esculenta), Radish (Raphanus sativus), Rutabaga (Brassica napus Napobrassica group), Salsify (Tragopogon porrifolius), Scorzonera (Scorzonera hispanica), Skirret (Sium sisarum), Sweet Potato or Kumara (Ipomoea batatas), Taro (Colocasia esculenta), Ti (Cordyline fruticosa), Tigernut (Cyperus esculentus), Turnip (Brassica rapa Rapifera group), Ulluco (Ullucus tuberosus), Wasabi (Wasabia japonica), Water chestnut (Eleocharis dulcis), Yacón (Smallanthus sonchifolius), Yam (Dioscorea spp.)]; spices and other flavorings [e.g., ajowan (Trachyspermum ammi) allspice (Pimenta dioica), amchur (Mangifera indica), angelica (Angelica spp.), anise (Pimpinella anisum), annatto (Bixa orellana), asafoetida (Ferula asafoetida), barberry (Berberis spp (many) and Mahonia spp (many)), basil (Ocimum spp), bay leaf (Laurus nobilis), bee balm (bergamot, monarda; Monarda spp.), black cumin (Bunium persicum), black lime (loomi; Citrus aurantifolia), boldo (boldina; Peumus boldus), bush tomato (akudjura; Solanum central), borage (Borago officinalis), calamus (sweet flag; Acorus calamus), candlenut (Aleurites moluccana), caraway (Carum carvi), cardamom (Amomum compactum), capers (Capparis spinosa), cassia (Cimmanmomum cassia), cayenne pepper (Capsicum annum), celery (Apium graveolens), chervil (Anthriscus cerefolium), chicory (Cicorium intybus), chile/chili/chilli (e.g., Capsicum frutescens), chile varieties (Capsicum frutescens), chives (Allium odorum, Allium shoenoprasum), cilantro (Coriandrum sativum), cinnamon (Cinnamomum zeylanicum; Cinnamomum cassia), clove (Syzygium aromaticum), coriander (Coriandrum sativum), cubeb (Piper cubeba), cumin (Cuminum cyminum), curry leaf (kari; Murraya koenigii), dill (Anethum graveolens), elder (elder flower, & elderberry; Sambucus nigra), epazote (Chenopodium ambrosioides), fennel (Foeniculum vulgare), fenugreek (Trigonella foenum-graecum), galangal (Alpinia galangal), garlic (Allium sativum), ginger (Zingiber officinale), hoja santa (Piper auritum), horseradish (Armoracia rusticana), hyssop (Hyssopus officinalis), jamaican sorrel (Hibiscus sabdariffa), juniper (Juniperus communis), kaffir lime (Citrus hystrix), mustard (Brassica nigra), kokum (Garcinia indica), lavender (Lavandula angustifolia), lemon balm (Melissa officinalis), lemon grass (Cymbopogon citrates), lemon myrtle (Backhousia citriodora), lemon verbena (Lippia citriodora), licorice (Glycyrrhiza glabra), lovage (Levisticum officinale), mace (Myristica fragrans), mahlab (Prunus mahaleb), marjoram (Majorana hortensis), mastic (Pistacia lenticus), melegueta pepper (Aframomum melegueta), grains of paradise (Aframomum granum paradise), mint (Mentha spp.), mountain pepper (Tasmannia lanceolata), Tasmanian pepper (Tasmannia lanceolata), myrtle (Myrtus communis), nigella (Nigella sativa), nutmeg (Myristica fragrans), onion (Allium cepa), orris root (Germanica florentina), paprika (Capsicum annuum), parsley (Petroselinum crispum), pepper (Piper nigrum), poppy seed (Papaver somniferum), rosemary (Rosmarinus officinalis), saffron (Crocus sativus), sage (Salvia officinalis), sassafras (Sassafras albidum), savory (Satureja hortensis), scented geranium (Pelargonium spp), screw-pine (pandan; Pandanus tectorius), sesame (Sesamum indicum), soapwort (Saponaria officinalis), sorrel (Rumex acetosa), star anise (Illicium verum), sumac (Rhus coriaria), szechwan pepper (Zanthoxylum spp. (piperitum, simulans, bungeanum, rhetsa acanthopodium)), tamarind (Tamarindus indica), tarragon (Artemisia dracunculus), thyme (Thymus vulgaris), turmeric (Curcuma longa), vanilla (Vanilla planifolia), wasabi (Wasabia japonica), watercress (Nasturtium officinale), wattleseed (Acacia aneuro), zedoary (Curcuma zedoaria), and sea vegetables [e.g., Aonori (Monostroma spp., Enteromorpha spp.), Carola (Callophyllis variegata), Dabberlocks aka badderlocks (Alaria esculenta), Dulse (Palmaria palmata), Gim (Porphyra spp.), Hijiki (Hizikia fusiformis), Kombu (Laminaria japonica), layer (Porphyra spp.), Mozuku (Cladosiphon okamuranus), Non (Porphyra spp.), Ogonori (Gracilaria spp.), Sea grape (Caulerpa spp.), Seakale (Crambe maritima), Sea lettuce (Ulva lactuca), Wakame (Undaria pinnatifida)], some of which are not plants in the taxonomic sense.

Also contemplated are mistletoes and other saprophytic organisms, some of which are recognized as producing medically important metabolites.

Of particular interest for the method described herein are berry fruits, which are already recognized as producing a wide array of (beneficial) phytochemicals. The botanical definition of a berry is a simple fruit produced from a single ovary, such as a grape. The berry is the most common type of fleshy fruit in which the entire ovary wall ripens into an edible pericarp. The flowers of these plants have a superior ovary formed by the fusion of two or more carpels. The seeds are embedded in the flesh of the ovary. However, the term “berry” as used herein is broader than the botanical definition and encompasses, for instance, false berries (e.g., blueberries), aggregate fruits (e.g., blackberries and raspberries), drupes (e.g., hackberries and Açaí palm), and accessory fruits (e.g., strawberries).

Examples of true berries include: grape (Vitis vinifera), tomato (Lycopersicon esculentum and other species of the family Solanaceae, many of which are commercial importance, such as Capsicum, and aubergine/eggplant (Solanum melongena), wolfberry or Goji berries (Lycium barbarum, Lycium spp.; Solanaceae), garberry (Berberis; Berberidaceae), red, black, and white currant (Ribes spp.; Grossulariaceae), elderberry (Sambucus niger; Caprifoliaceae), gooseberry (Ribes spp.; Grossulariaceae), honeysuckle (Lonicera spp.; Caprifoliaceae) (the berries of some species (e.g., honeyberries) are edible, and even though others are poisonous they may provide useful phytochemicals if properly purified), mayapple (Podophyllum spp.; Berberidaceae), nannyberry or sheepberry (Viburnum spp.; Caprifoliaceae), Oregon-grape (Mahonia aquifolium; Berberidaceae), and sea-buckthorn (Hippophae rhamnoides; Elaeagnaceae). Also contemplated herein within the term “berries” are the modified, juicy berries, such as the fruit of citrus. Such fruits, including orange, kumquat, grapefruit, lime, and lemon, are modified berries referred to botanically as hesperidium.

Also specifically contemplated herein is the chokeberry (Aronia melanocarpa; commonly called black chokeberry), which has attracted scientific interest due to its deep purple, almost black pigmentation that arises from dense contents of phenolic phytochemicals, and especially anthocyanins. Total anthocyanin content in chokeberries is 1480 mg per 100 g of fresh berries, and proanthocyanidin concentration is 664 mg per 100 g (Wu et al., J Agric Food Chem. 52: 7846-7856, 2004; Wu et al., J Agric Food Chem. 54: 4069-4075, 2006). Both values are among the highest measured in plants to date. Chokecherry produces these pigments mainly in the skin of the berries to protect the pulp and seeds from constant exposure to ultraviolet radiation (Simon, HortScience 32(1):12-13, 1997). By absorbing UV rays in the blue-purple spectrum, pigments filter intense sunlight. Scientific measurement of ORAC antioxidant strength demonstrates chokeberry with one of the highest values yet recorded—16,062 micromoles of Trolox equivalents per 100 g (Nutrient Data Laboratory, Agriculture Research Service, US Department of Agriculture, 2007 publication entitled “Oxygen Radical Absorbance Capacity (ORAC) of Selected Foods,” available on-line; see this ORAC reference also provides antioxidant scores for 277 common foods). Analysis of anthocyanins in chokeberries has identified the following individual chemicals (among hundreds known to exist in the plant kingdom): cyanidin-3-galactoside, epicatechin, caffeic acid, quercetin, delphinidin, petunidin, pelargonidin, peonidin, and malvidin. All these are members of the flavonoid category of antioxidant phenolics, and they are found in myriad other plants in differing concentrations.

Many “berries” as referenced herein are not true berries by the scientific definition, but are in fact drupes, epigynous fruits, or compound fruits. Drupes are fruits produced from a single-seeded ovary or achene; example drupes are hackberry (Celtis spp ; Cannabaceae) and Açaí (Euterpe), a palm fruit native to the Amazon region. Epigynous fruits are berry-like fruits formed from inferior ovaries, in which the receptacle is included. Notable examples are the fruits of the Ericaceae, including blueberry, huckleberry, and cranberry. Other epigynous fruits include: bearberry (Arctostaphylos spp.), crowberry (Empetrum spp.), lingonberry (Vaccinium vitis-idaea), strawberry tree (Arbutus unedo), and sea grape (Coccoloba uvifera; Polygonaceae). The fruit of cucumbers, melons and their relatives are modified berries called “pepoes.” Compound fruits are groups or aggregates of multiple individual fruits with seeds from different ovaries of a single flower, and include: blackberry, dewberry, boysenberry, olallieberry, and tayberry (genus Rubus), cloudberry (Rubus chamaemorus), loganberry (Rubus loganobaccus), raspberry, Rubus idaeus and other species of Rubus, salmonberry (Rubus spectabilis), thimbleberry (Rubus parviflorus), wineberry (Rubus phoenicolasius), bayberry, and boysenberry. Multiple fruit are the fruits of separate flowers, packed closely together, such as the mulberry. Others are accessory fruit, where the edible portion is not generated by the ovary, such as the strawberry.

Berry colors are due to natural plant pigments. Many are polyphenols such as the flavonoids, anthocyanins, and tannins localized mainly in berry skins and seeds. Berry pigments are usually antioxidants and thus have oxygen radical absorbance capacity (“ORAC”) that is high among plant foods (Wu et al., J. Agric. Food Chem. 52(12):4026-4037, 2004). Together with good nutrient content, ORAC distinguishes several berries within a new category of functional foods called “superfruits” and is identified by DataMonitor as one of the top 10 food categories for growth in 2008 (Food Navigator—USA.com, “Fresh, super and organic top trends for 2008”, Nov. 28, 2007).

Particularly contemplated plants include coffee, tea, blueberry, cranberry, c. buckthorn, cannabis, sequoia, grape, huckleberry, orchid, frankincense, ficus, blackberry, black olive, red currant, raspberry, spinach, red pepper, chili pepper, and beetroot. Plants (or other organisms) recognized as having anti-aging properties are also particularly contemplated, including but not limited to long-aged plants (e.g., the Malus domestica (Apple) cultivar Uttwiler Spaetlauber described in U.S. Patent publication no. 2008/0299092), plants used in ancient medicine for improving health or longevity, and so forth.

Additional sources for modulating compounds, and methods for preparing compositions containing such, can be found in the literature. See, for instance, European published application EP 1,985,280; Schmid et al., “Plant Stem Cell Extract for Longevity of Skin and Hair” SOFW-Journal, 134:30-35, 2008; U.S. Pat. Nos. 7,544,497, 7,582,674; and International Patent Publication No. WO/2007/084861.

Further exemplary modulating compounds include for instance stress-induced phenylpropanoids (see, e.g., Dixon et al., The Plant Cell 7:1085-1097, 1995).

Exemplary metabolite compounds or agents include those selected from the group of compounds contained in coffee cherry acids or extracts including the antioxidant compounds chlorogenic acid, quinic acid, caffeic acid, ferulic acid and proanthocyanidins.

Exemplary metabolite compounds or agents include ubiquinone, idebenone and the analogs and derivatives thereof including various esters and conjugated compounds.

Exemplary modulating metabolite compounds or agents include extracts and the analogs and derivatives obtained from cocoa. The extracts, compounds or combinations of compounds derived from the cocoa beans from various isolation or purification processes are derived from any species of Theobroma, Herrania or inter- or intra-species hybrid crosses thereof. It is also understood that similarly such extracts or compounds are included if derived from genetically engineered versions of these species or hybrids. Furthermore synthetic formulations, analogs or derivatives of these compounds are similarly included as well as compounds derived from natural or synthetic fermentation processes. These extracts or compounds preferably comprise polyphenol(s) such as cocoa procyanidin(s), such as at least one cocoa procyanidin selected from (+) catechin, (−) epicatechin, procyanidin oligomers 2 through 18, procyanidin B-5, procyanidin B-2, procyanidin A-2 and procyanidin C-1.

Exemplary modulating compounds or agents include extracts and the analogs and derivatives obtained from Camellia sinensis, Camellia sinensis sinensis, Camellia sinensis assamica or Camellia oleifera either naturally or synthetically derived.

Exemplary metabolite compounds or agents include resveratrol and the analogs and derivatives thereof, including viniferin, gnetin H, and suffruticosol B.

Conventional methods of preparing (green) tea extracts, see, for instance, Perva-Uzunalic et al., Food Chemistry 96(4):597-605, 2006; Koiway & Masuzawa, Jpn. J. Appl. Phys 46:4936-4938, 2007; U.S. Pat. Nos. 4,668,525 and 3,080,237. Tea extracts containing polyphenols, as well as individual tea-derived polyphenols, are commercially available from many sources. By way of example only, one source is Pharma Cosmetix Research, LLC (Richmond, Va.), the supplier of Premier Green Tea Extract Lot #10783 that was used in various examples described herein.

For the sake of comparison or reference, idebenone (CAS no. 58186-27-9) is commercially available from myriad suppliers, including for instance Pharma Cosmetix Research, LLC (Richmond, Va.). Likewise, coffee cherry extract for reference or comparison can be prepared using art recognized methods; see, for instance U.S. Patent Publication No. 2007/0281048 (published Dec. 6, 2007). In addition, the coffee cherry extract referred to as COFFEEBERRY® can be purchased from VDF FutureCeuticals, Inc. (Momence, Ill.).

Additional descriptions of compounds or products from plants that can be adapted for production in the systems described herein include: U.S. Publications No. 2005/0129827, 2005/0096256, 2005/0197407, 2003/0161902, 2003/0082117, and 20070281048, and U.S. Pat. Nos. 6,989,150 and 6,670,390.

Particularly contemplated are comparative tissue cultures from different but related species or even cultivars, for instance Coffea arabica versus robusta. With this particular example, one is naturally 4N (that is, polyploidy) while the other is 2N—and the resultant genetic expression is different between these two species. As evolution adaptation occurred, the biochemistry of plants has changed to enable their survival and allow them to thrive in new conditions. The ratio and likely in many cases the actual chemical production of the different species will vary. The methods provided herein allow evaluation of, and exploitation of, such evolved differences.

Also contemplated are manufactured mutations—including changes in ploidy (for instance, due to colchicine treatment), genetically altered cells (for instance, transformed with expression vector(s) that expression one or more heterologous genes), cells that have one or more native genes knocked out, and so forth. The modifications can be made before or after the cells are placed in cell culture—that is, the plant from which the cells are derived can be genetically altered, or isolated cells can be altered genetically. Any conventional means for altering the genetic makeup of a cell can be employed.

Also contemplated are tissue cultures produced from natural or artificial hybrids, such as for instance intergeneric hybrids (e.g., the tangelo).

Also contemplated herein are methods of using non-plant organisms, such as algae, bacteria or fungi, for production metabolite chemicals in culture. For instance, protist cultures are contemplated—including plant-like protists (algae, particularly green, brown and red algae) and animal like protists (protozoa). By way of example, algae and plankton (including marine plankton) are known to produce DNA repair enzymes (e.g., photolyases, produced for instance by Anacystis nidulans; see, e.g., The New Science of

Perfect Skin: Understanding Skin Care Myths and Miracles For Radiant Skin at Any Age, by Dr. Yarosh; Broadway Books 2008; ISBN 978-0-7679-2539-6), and are believed to be amenable to exploitation in tissue culture. Likewise, diatoms can be exploited for instance in order to harvest their nanoscale shells/skeletons, which are useful in myriad products. Also contemplated are cultures of fungal cells, including unicellular fungi (such as yeasts) and multicellular fungi (including mushrooms, molds, and so forth). Many beneficial compounds are recognized as being produced by mushrooms, including for instance pigment lightening compounds such as kojic acid and proteins (such as tyrosinase) and other metabolites that impact melanin biosynthesis. All three main categories of fungi (thread-like (mold), sac-like (yeast) and clublike (mushroom)) are contemplated. One specific example mushroom that is contemplated for use herein is Trametes versicolor (formerly Coriolus versicolor and Polyporus versicolor), commonly referred to as Turkey Tail; this mushroom produces, for instance, polysaccharide-K (Krestin, PSK), a recognized immune system boosting agent that has been used in cancer treatment.

Also contemplated are embodiments that exploit bacteria in culture in order to produce metabolites. For instance, cyanobacteria are of interest for several reasons—they are photosynthetic and they are more algae-like than other bacteria; they have ability to produce various toxins that may be exploited for clinical or other uses (e.g., similarly to botulinum toxin). Certain cyanobacteria produce cyanotoxins including anatoxin-a, anatoxin-as, aplysiatoxin, cylindrospermopsin, domoic acid, microcystin LR, nodularin R (from Nodularia), or saxitoxin. These toxins can be neurotoxins, hepatotoxins, cytotoxins, and endotoxins. Recent genomic analyses of cyanobacteria have revealed a surprisingly large number of toxin-antitoxin loci in free-living prokaryotes. The antitoxins are proteins or antisense RNAs that counteract the toxins. Two antisense RNA-regulated toxin-antitoxin gene families, hok/sok and ldr, are unrelated sequence-wise but have strikingly similar properties at the level of gene and RNA organization. Recently, two SOS-induced toxins were found to be regulated by RNA antitoxins. One such toxin, SymE, exhibits similarity with MazE antitoxin and, surprisingly, inhibits translation.

### VI. Using In Vitro (Tissue) Culture to Produce Dedifferentiated/Stem Cells

In general, the methods of culturing tissues used herein are conventional and appropriate tissue culturing systems will be recognized by those of skill in the art. The art is rife with descriptions of how to culture plant cells in vitro, and how and what factors can be varied in order to obtain healthy cultures of specific plant cells or tissue types. By way of example, the following are recognized textbooks that provide teachings in this areas: Plant Cell and Tissue Culture, a Laboratory Manual, Reinert & Yoeman, Springer-Verlag 1982 (ISBN 3-540-11316-9); Plant Tissue Culture Propagation, de Fossard, Filmfiche Corp. 1981 (ISBN 0-949801-003); In Vitro Culture of Higher Plants, Pierik, Martinus Nijhoff Publishers 1987 (ISBN 90-247-3531-9); Plant Culture Media, Volume 1, Formulations and Uses, George, Puttock & George, Exegetics Ltd. 1987 (ISBN 0-9509325-2-3); Plant Cell Culture Secondary Metabolism: toward industrial application, DiCsomo & Misawa, CRC Press LLC 1996 (ISBN 0-8493-5135-9); Plant Tissue Culture: Theory and Practice, a revised edition, Bhojwani & Rasdan, Elsevier 1996 (ISBN 0-444-81623-2); Plant Cell Culture Protocols, 2^{nd} Ed., Loyola-Vargas & Vazquez-Floga, Humana Press 2006 (ISBN 1-59259-959-1); and so forth.

The culture media which can be used according to the teachings herein are those which are generally known to one skilled in the art. Examples that can be cited include the media of Gamborg, Murashige and Skoog, Heller, White etc., examples of which are well known. Complete descriptions of these media are given in “Plantation Culture Media: Formulations and Uses” by E. F. George, D. J. M. Puttock and H. J. George (Exegetics Ltd. 1987, Volumes 1 & 2).

With the knowledge available in the art, and the teachings provided herein, there are now enabled methods for producing metabolite-producing in vitro culture systems. In particular embodiments, the cells in culture are fully undifferentiated (or dedifferentiated), partially differentiated, or in some instance fully differentiated. The art recognizes methods for producing specific levels of differentiation (or dedifferentiation) in various species of plants and other organisms. The teachings herein also provide systems for empirical testing, for instance systems for testing cell types that are not discussed in the art.

As described herein, the organisms from which cell cultures are contemplated are myriad, as are the tissues that are selected for culture. It is believed that different organisms (e.g., different species, different cultivars), organisms that originate in different places, different tissues from the organisms that are used for culture, and of course how the tissues are treated in culture are all likely to have impacts on the type, mix, and level of metabolite(s) that are produced in the cultures.

Cultures of dedifferentiated cells are known, as are the mechanisms of elicitation of these cells followed by extraction stages and by various filtrations followed by freeze-drying in order to incorporate the extracts obtained in a cosmetic or pharmaceutical preparation. Such methods are described, for example, in U.S. Pat. No. 4,241,536; U.S. published application 2005/0265953, EP 378 921, WO 88/00968, EP 1 203 811, and so forth for species of various plants. The content of these documents is incorporated in the present description by reference in order to describe culture media, plant species, possible elicitors, etc.

Suspension cultures can be raised from the callus cultures and maintained in fresh suspension medium. Suitable nutrient media for plant cell suspension culture are well known to one of skill in the art. In a particular example, a plant cell suspension culture medium includes Murashige and Skoog (MS) salts (e.g., Cat. No. M524, Phytotech, Shawnee Mission, Kans.) and Nitsch and Nitsch vitamins (e.g., Cat. No. N608, Phytotech, Shawnee Mission, Kans.). See, e.g., Nitsch and Nitsch, Science 163:85-87, 1969. Suspension cultures can be established by aseptically transferring a known mass of cells expressed as packed cell volume (PCV) to fresh medium on a regular schedule, typically at 7-14 day intervals.

Medium for suspension culture can be optimized for initiation of suspension culture or for desired characteristics (such as cell texture or plant metabolite production). In some examples, the concentration of hormones (such as 2,4-D, dicamba, NAA, 6-γ-γ-dimethylallyl-aminopurine (2iP), picloram, indole-3-acetic acid (IAA), gibberellic acid (GA), or kinetin) can be varied individually or in combination. In particular examples, the medium may contain about 0-2 mg/L 2,4-D (for example, about 0 about 0 mg/L, 0.005 mg/l, 0.01 mg/L, 0.05 mg/L, or 0.1 mg/L), about 0-2 mg/L dicamba (for example, about 0 mg/L, 1 mg/L, or 2 mg/L), about 0-1 mg/L GA (for example, about 0 mg/L, 0.5 mg/L, or 1 mg/L), about 0-2 mg/L IAA (for example, about 0 mg/L, 1 mg/L, or 2 mg/L), about 0-1 mg/L kinetin (for example, about 0 mg/L, 0.1 mg/L, 0.5 mg/L, or 1 mg/L), about 0-2 mg/L NAA (for example, about 0 mg/L, 0.5 mg/L, 1 mg/L, or 2 mg/L, about 0-2 mg/L picloram (for example about 0 mg/L, 1 mg/L, or 2 mg/L), about 0-0.1 mg/L BA (for example, about 0 mg/L, 0.01 mg/L, 0.02 mg/L, 0.025 mg/L, 0.05 mg/L, or 0.1 mg/L), or various combinations of one or more thereof.

It is specifically recognized that sterile coconut milk may be useful as a component in cell culture as described herein. Coconut milk/water has long been a recognized source of auxin as well as sugars, minerals, vitamins, etc. Freshly harvest coconut milk, particularly from green coconuts, is advantageous. Sterile coconut milk is commercially available (see, e.g., Coconut Water, Catalog #C5915 from Sigma Aldrich).

It is expected that production of the cellular materials (e.g., extracts, metabolites, and so forth) will be from liquid cultured callus tissue, for instance grown in a bioreactor or other vessel. This allows high production level, enables scale up (for instance using larger or more bioreactor tanks), and allows for ongoing harvest (e.g., feed through culture rather than batch culture). It is expected that in some embodiments, the biomass in the bioreactor is not the source of the desired metabolite material, but rather the metabolite(s) are continuously harvested from the reactor—such as the medium, or a fraction of the biomass, leaving the remaining biomass in the reactor to continue to produce material. In those embodiments where the cultured biomass itself contains the target material, the biomass will need to be fractionated or otherwise extracted—and the liquid culture permits removal of a substantial portion of the biomass for processing while keeping the culture ongoing. The removed biomass is eventually replaced through proliferation, and the resultant biomass is removed in an on-going cycle.

Solid medium culture is less often used, though it is expected to be useful in those embodiments biomaterial target must be harvested from the sterile plant/plantlet itself (for example apical meristem that underwent a change in callus tissue to render the required material inert, or decreased production to an unharvestable level). Another instance where solid medium is beneficial is where the volumes required are very small. In such cases, callus tissue can be taken straight from the subcultures without the need for suspension culture expansion. A final use for solid culture is when the target bioproduct comes from the root tips, roots or fruit of the plant. In that case sterile cultures would need to be generated on media to ensure pathogen free product. However, it is also contemplated that appropriate culture conditions can be devised to produce normally root-products even in suspension culture. Also contemplated are temporary immersion systems for in vitro plant culture, such as a RITA® (Cirad, France) system.

### VII. Elicitation/Stimulation of Cells in Culture

The use of elicitors to stimulate metabolite formation and secretion is an important tissue culture process strategy. Elicitation/stimulation has been very useful to reduce the process time necessary to reach a high product concentration. Further, elicitation may result in the formation of novel compounds (see, e.g., Payne et al., Plant Cell and Tissue Culture in Liquid Systems, pp 333-351, 1995, New York, John Wiley & Sons, Inc). Optimization of elicitation with biotic and/or abiotic candidate elicitors can be tested in small- or large-scale reactor culture to optimize when (and what) elicitors can and should be used, what dosage can be the best, how long the cells should be exposed to the eliciting factors, and when the cells should be harvested, in order to optimize production of the desired metabolite(s). Synergistic effect using multiple treatments of elicitors can also be evaluated, to identify combinations that enhance productivity of specific metabolites.

Elicitor exposure if multiple can be simultaneous, sequential, alternating, or any other combination—the duration of exposure as well as the time interval between exposures and the total number of exposures all can be varied, as can the sequence in which elicitors are applied. There can also be (for single elicitors or co-elicitation) a ‘duty cycle’ of exposures. Light is one example, but could the concentration(s) of elicitor(s) can in various embodiments wax and wane, thus modulating the elicitor exposure with single (or multiple) elicitors. Elicitors may also influence the expression of more than one gene, as clearly illustrated herein with example patters of expression. Exploiting this can yield maximum production of one target product (either cellularly or secondary metabolic products); it can also create a mixture of target metabolites by first eliciting one response then eliciting another—such that sequential elicitor protocols emerge to produce customized blends of biologically active compounds. This fine tuning of elicitation can also be used to reduce the production of undesirable substances in the culture.

Thus, optionally, cells in culture are subjected to an elicitation step prior to extraction or harvesting of metabolite(s) from the culture. Benefits of elicitation have been recognized in whole plants (see, e.g., Kuzel et al., J. Agric Food Chem 57(17):7907-7011, 2009). Biotic stimulation has been exploited by some research groups (see, e.g., USDA Research Project 6435-53000-001-00, to Boue & Bhatnager, describing induction of isoflavonoids in legumes using A. sojae or A. sojae cell wall extracts). U.S. Pat. No. 5,935,809 describes methods of inducing plant defense mechanisms, for instance using which may be jasmonic acid, lower alkyl esters of jasmonic acid or jasmonic acid-like derivative compounds to induce the expression of genes in the plants resulting in the production of defense proteins, such as proteinase inhibitors, thionins, chitinases and β-glucanases. Kuzel et al. (J Agric Food Chem. 57(17):7907-11, 2009) describe the elicitation of pharmacologically active substances in an intact medical plant, Echinacea purpurea (purple coneflower).

Thus, the concept of ‘selective elicitation’ or ‘selective biosynthesis’ can be exploited in the systems provided herein, to target the production of one or more (often complex and possibly not able to be currently synthesized) chemicals. A possible analogy is to view genes as keys on piano, and elicitation as a method of hitting keys. Depending on the elicitor(s) used, different combinations of keys are played—and systems can be developed empirically to play chords—to obtain specific induction of gene products and thereby metabolites or sets of metabolites.

In other embodiments, the elicitor is an abiotic stress, such as UV light or other irradiation exposure, which is used to elicit the production of ‘defense chemicals’—some of which may be involved in DNA repair while some prevent apoptosis.

The items listed as variables for optimizing or altering cell culture can also be applied here as elicitation agents.

By way of example, yet more elicitation influences include altered (e.g., low intensity or very low intensity) light exposure, which can be used to photobiomodulate gene expression, exposure to microwaves, ultrasonic waves, radiofrequency, infrared radiation, ionizing radiation, visible light of specific wavelengths, and so forth.

Also contemplated are systems where the cell cultures are stressed, then cells are selected for those which are mutated in order to adapt to the stress. Such cells can then be used for altered production of biomolecules, or to generate completely differentiated tissues, up to and including whole plants for agriculture.

Present in more or less large amounts in all the organs of the plant, phytoalexins can be induced in leaves and berries. This type of induction is designated by the term elicitation. Elicitation factors (or elicitors) have many different origins, and elicitation can take the form of: biotic elicitation, for example on an attack by a pathogen such as Botrytis cinerea, a grey rot agent; Plasmopara viticola, a mildew agent; or Phomopsis viticola, which is responsible for excoriosis; and abiotic elicitation by environmental factors such as UV or other irradiation, temperature, light, asphyxia, natural agents extracted from other plants, aluminum chloride, ozone and many other factors.

On elicitation, phytoalexins such as trans-resveratrol, trans-piceid, .epsilon.-viniferin and pterostilbene can be induced in leaves and berries. This property of the de novo biosynthesis of phytoalexins in response to a stress, particularly after attack by a pathogen, suggests that these molecules could play the role of natural means of defense of the plants. This role of defense molecules is corroborated by certain studies which seem to indicate a close interrelationship between the level of natural resistance of the plant and its ability to synthesis these molecules. For example, Langcake and McCarthy (Vitis 18(3):244-253, 1979) demonstrated a relation between the resistance of certain species of the Vitis kind to Botrytis cinerea or Plasmopara viticola and their capacity for the biosynthesis of phytoalexins (resveratrol and viniferin). Moreover, Dercks and Creasy (Physiol Mol Plant Path. 34:189-202, 1989) showed that species resistant to Plasmopara viticola produce five times more phytoalexins than do sensitive species. Similarly, within the Vinifera species there are some vines which are more or less tolerant to attack by fungi depending on their capacity for producing phytoalexins.

Cell elicitation can be effected by means of agents or by means of various stresses, such as pressure, depressurization, vacuum, pressure variations, the presence of a gas, a variable atmosphere, temperature, cold, light intensity or spectral distribution or ratio or cycle of brightness, radiation, a toxin, a plant toxin, a plant extract other than a toxin, an antioxidant or blend thereof, agitation, a bacterium, a virus, fungi, a microorganism, ultrasound, IR, UV, asphyxia, etc. Any method of elicitation known to one skilled in the art can be used to stimulate tissue cultures as described herein. It is further contemplated that other organic or inorganic chemicals/substances (including elements) may be useful to influence expression (and/or metabolite production) in the cell culture.

Callus and suspensions cultures may produce the desired natural product naturally or spontaneously. However, at times, though the host plant produces the natural product, the callus and/or suspension cultures may not, or may produce the natural product at lower levels than in the host plant. Methods of inducing or increasing the production of a natural product are known to one of skill in the art. For example, cultures can be induced to produce anthocyanins using light irradiation, especially ultraviolet (UV)-B light which is an elicitor of anthocyanin biosynthesis (Reddy et al., 1994, Plant Physiol. 105: 1059-1066).

Additional treatments/stressors/elicitation events include: exposure to ultraviolet radiation (full spectrum or specific wavelengths, such as UVA 1, UVA2, UVB, UVC, etc. . . . ), Hydrogen Peroxide addition, Low nutrient media/“caloric” restriction, increased/decreased temperature, overexposure or removal of light, hyper/hypoxia, mechanical stress (e.g., photoacoustic shock waves, ultrasonic shock waves, violent stirring), exposure to blue light in the 400-475 nm range, exposure to other wavelengths of light either singularly or in concert, exposure to other chemicals, insect or herbivore saliva or a component thereof, a photosome or ultrasome, and so forth. Thus, the following are considered elicitors (or eliciting events): specific wavelength(s) of light; electromagnetic radiation electrical current/potential ionizing radiation high or low light intensity; nitrogen source limitation; carbon source limitation; phosphorus source limitation; water limitation; high salt exposure; high temperature exposure; low temperature exposure; contact stress or wounding; a pathogen-derived compound; a pesticide; a herbicide; a fungicide; a bactericide; anti-viral agent; wounding; a microbial (bacterial, viral, fungal) pathogen or fraction thereof; a nematode or fraction thereof; peroxide; an enzyme; a chemical; a fatty acid; an amino acid; saliva from herbivorous insect or other animal;

vibration; gravity or lack thereof, or reduced or increased gravitational field; an extract from a plant; cAMP; ethylene or another gas; and/or a transformation vector (that results in expressing an eliciting compound or protein).

Also contemplated are less direct forms of “elicitation”, including for instance engineered self-elicitation whereby a cell (in culture) is expresses (or more particularly, has been engineered to express) a (heterologous) gene that encodes a factor which elicits a response from the cell or the culture as a whole when the factor is expressed. Methods of transforming cells (including plant, bacterial, and fungal cells) are well known in the art. By way of example, it is contemplated to culture cells that have been engineered to express one or more of: a harpin (see, e.g., U.S. Pat. Nos. 7,132,525; 6,583,107; WO 98/054214), a photolyase (see, e.g., Kao et al., PNAS 102(45): 16128-16132, 2005), an antioxidant compound not normally expressed by the cell (or not normally expressed at that level), an isoflavone, a phytoalexin, a cytochrome P450 (e.g., a Soybean or Medicago truncatula CYP93C gene), a protein involved in phenylpropanoid metabolism (see, e.g., U.S. Pat. No. 7,129,088, and so forth.

### VIII. Optimization and Up-Scaling of Tissue Culture and Growth Procedures

It will be recognized by those of skill in the art that tissue culture conditions can be optimized, for instance for specific plants or other species, specific plant tissues, the production of specific compounds or extracts, and so forth. Conventionally, optimization of tissue culture involves evaluation of cell production (e.g., biomass volume or weight, cellular health, optical density, and so forth), bi-product or metabolite production (amounts, concentrations, constituents, purity, and so forth), and other physical evaluations. The systems described herein provide additional ways to evaluate “optimal” tissue culture conditions—by evaluating the biological effects of extracts or metabolites produced from the cultured cells.

While one of skill in the art will recognize what components and other factors can be varied in order to test and evaluate optimized tissue culture conditions. The following are therefore provided simply as sample factors, rather than limiting sets of variables that can be modified during an optimization process.

Large-scale plant cell culture is important technology in the development of a commercial process. This can be performed in large tanks similar to those used in microbial fermentation. Productivity enhancement in these tanks can be achieved by determining biomolecular factors based on cellular growth and production characteristics in the large scale process and by optimizing large-scale bioprocess variables that enhance procyanidin productivity. Biomolecular factors include medium components, elicitors and precursors in biosynthetic pathway. Prior to large-scale process, these factors can be examined in small (e.g., flask-scale) process, since the goal of scale-up process is to reproduce on a large scale those conditions observed to be optimal on the smaller scale. However, conditions in large-scale bioreactor culture can be different to specific cell types, or tissue types, or plant cell sources, suspended cells in flask-scale culture. The macrokinetics of the culture are affected by changes in environmental conditions affecting the suspended cells. For instance, while parameters of growth kinetics are scale independent, the overall growth of a cell culture in a vessel is scale dependent because of the scale dependency on transport of gaseous and dissolved nutrients and metabolites (Dicosmo & Misawa, Plant Cell Culture Secondary Metabolism, pp 11-44, 1996. Boca Raton, Fla., CRC Press LLC). Therefore, in scale up of the bioreactor process for metabolite production, a number of basic experiments will be performed to produce data including growth rate, product formation rate, nutrient uptake rate and respiration rate.

In general, high productivity in plant cell cultures can be achieved by increasing the cell concentration and/or specific productivity. The maximum cell concentration is influenced by nutrient supply, yield of biomass per substrate and water content. Additional environmental factors can be varied one by one or multiple factors can be varied at one time to increase biomass. Bioprocess variables such aeration rate, rheological properties of suspension cultures affect mass transfer and mixing in bioreactors. This can have a strong impact on production of plant metabolites. The variables of aeration rate, agitation speed, type of mixing impeller, other mixing-related variables and even medium composition can be optimized separately for growth stage and production stage. However, it is unlikely that all conditions can be kept completely optimal in a scale up a process. Choices have to be made as to which variable is considered as the most important; such decisions are within the ordinary skill of the artisan working in this field.

The effects of supplementing carbon and nitrogen sources on growth and production are also studied based on basic engineering data of carbon and nitrogen consumption, since the relative amounts of carbon and nitrogen sources play an important role in enhancing the biosynthesis of metabolites and cell growth (Basaria, Current Biology, 2: 370-374, 1990). Supply of oxygen and carbon dioxide can also be examined. In addition to oxygen, carbon dioxide has been reported to improve cell growth and secondary metabolite production in plant cell cultures (Thanh et al., Biologia Plantarum, 50: 752-754, 2006; Tate & Payne, Plant Cell Reports, 10: 22-25, 1991). Oxygen requirements of plant cells are relatively low in cell growth stage, but may significantly increase during metabolite synthesis. The level of these gases provided to the cell system can be controlled for their optimal utilization in culture.

In large scale fermentation, it is impossible to introduce the same amounts of gas (air, oxygen etc.) as can be introduced on a laboratory scale. Therefore, it can be beneficial to maintain the mass transfer coefficient constant, in order to make the superficial gas velocity constant during the bioreactor process.

Representative variables that are subject to optimization include: concentrations of hormones (e.g., auxins or cytokinins, or the ratio between them); concentrations and form of essential nutrients (e.g., carbon source such as sugar(s), nitrogen source, light exposure (level, wavelength, and duration, for instance), concentrations of salts, minerals, metals, vitamins, micronutrients and so forth, levels of dissolved gases (e.g., oxygen, carbon dioxide, air (a mixed gas), nitrogen), temperature at which the culture is maintained (or regimen of temperature changes to which the culture is subjected), solid versus liquid medium, and so forth.

The bioreactor or other cell growth vessel is also a variable that can be optimized, modified, and changed in order to get different results. For some embodiments, cells or tissue samples are cultured in a RITA® (Cirad, France) temporary immersion system for in vitro plant culture or equivalent system. Many variables impact how tissue cultures are treated in such a system, including for instance immersion frequency and duration, air flow rate/aeration, and the type of tissue subject to such growth (for instance, full plantlets, or callus tissue only). For methods of influencing plant tissue culture production using a temporary immersion system such as RITA®, see e.g., Pavlov & Bley (Process Biochemistry, 41(4), 848-852, 2006), Etienne and Berthouly (Plant Cell, Tissue and Organ Culture, 69:215-231, 2002), and International Patent Publication WO 2008/090435 and U.S. Patent Publication 2008/0176315 (Apparatus for Temporal Immersion Culture of Cells), and references discussed and/or cited therein.

Likewise, certain conditions can be varied no matter what type of bioreactor is used to grown the described cell cultures. These include, but are not limited to: CO₂ concentration, O₂ concentration (and/or plain air flow rate, for instance at normal O₂ levels), N₂ concentration, rotation/agitation speed of impellers, type of impeller (e.g., pitched blade impeller), air flow rate/aeration, duration tissue is in the bioreactor before transfer/use, when and how biomass generated by the reactor is replated to solid media before use (for instance, to enable plant tissue development, or partial or complete redifferentiation of cells), and simply the type of bioreactor or reactor vessel (e.g., glass jar, culture bag, wave bioreactor on a rocker, and so forth). Scale up of the tissue culture procedures provided herein is also within the skill of an ordinary practitioner. The requirements for scale up for tissue culture are influenced by the type of tissue/cells being cultured. Plant cell suspensions have a number of characteristics that are different from those of microbial cultures and which can affect their growth in bioreactors. Cultured plant cells are large (100 μm long), bound by a rigid cellulose-based wall and they often have a very large vacuole. Individual cells are rare, as cultures include mainly groups of cells or aggregates of 2 mm in diameter or above. Plant cells grow slowly (doubling times of 2-3 days) and consequently have a relatively low oxygen requirement.

Slow growth is one of the more important characteristics when considering bioreactor use for commercial applications. As a consequence of the slow growth, bioreactor runs can be as long as three weeks and longer—even up to three months or more, which reduces the number of runs possible, overall productivity of the system and requires strict maintenance of sterility. One method of increasing productivity is to increase the level of biomass. This can be achieved is several ways such as starting with a higher inoculum density, reducing the lag phase at the start of the culture by using actively growing cells as inoculum instead of stationary phase cells or using media compositions that enable faster cell growth. Using 2% (weight/volume) sucrose, plant cell cultures normally achieve biomass levels of around 10 g/L (dry weight). With a water content of 80-90%, the maximum biomass can be 90-100 g/L. In practice, biomass levels of 30-60 g/L (dry weight) would appear possible (Scragg, Curr Op Biotech 3:105-109, 1992). This is achieved not only by using nutrient rich media, but also appropriate bioreactor rheology conditions of aeration, agitation, gas mixing and so forth.

The high cell densities and the degree of aggregation may cause problems with both mixing and aeration, although the supply of oxygen may be less of a problem due to the low requirement. With a microbial culture, mixing at high biomass levels can be solved by increasing the impeller speed and power input. Plant cells, however, are generally sensitive to shear stress due to their size, cell wall and large vacuole (Taticek et al., Plant Cell 24: 139-158, 1991). This shear sensitivity has encouraged use of pneumatic reactors (also known as airlift bioreactors), which do not include any mechanical stirring arrangements for mixing. Alternatively, a number of bioreactor and impeller designs have been developed in order to produce good mixing with low shear stress. However, even at the relatively low aeration rate possible (0.1 vvm) pneumatic/airlift bioreactors can suffer from depletion of carbon dioxide and other components. Further, airlift bioreactors are not suitable for volumes above 100 L, making them unsuitable for large scale commercial production.

Mixing high levels of biomass has been investigated using various impeller designs. Hooker et al. (Biotechnol. Bioeng. 35: 296-304, 1990) compared a standard, 1.5 cm tall flat-bladed impeller with larger impellers (heights ranging between 5.1 cm and 14 cm). These were fitted in a 5 L Brunswick F5 bioreactor and a suspension culture of Nicotiana tabacum used as the test culture. The use of the largest (14 cm) flat-bladed impeller run at 150 rpm achieved the highest growth rate. A ‘cell-lift’ impeller that is supposed to generate lower shear rates than a Rushton turbine has been further modified by removal of the normal sparger from the Bio Flo II and replaced by direct sparging into the base of the impeller. This combination of cell-lift and airlift has been used to cultivate T. rugosum. By using perfusion-type culture (continuous replacement of the medium without loss of cells) a maximum biomass level of 27.6 g/L (dry weight) was achieved with no problems of mixing (Kim et al., Appl. Microbiol. Biotechnol. 34: 726-729, 1991).

Large scale cultivation of plant cells was previously restricted to bioreactors of up to about 100 L, though larger cultivations have been reported (e.g., Ritterhaus et al. (International Association for Plant Tissue Culture Newsletter 61: 2-10, 1990), reporting use of a cascade of bioreactors with volumes of 75, 750, 7500, 15,000 L to produce polysaccharides from Echinacea purpurea). Multiples of INTERMIG stirrers were used in these studies. These complex stirrers have the properties of low shear forces, good mixing, good dispersion of bubbles and low energy consumption. It is clear from this report that industrial scale growth of plant cells is possible and has been achieved.

In recent years there has been a shift away from capital-intensive stainless steel bioreactors to the use of disposable or single-use bioreactors. The development of flexible plastic containers supported by rigid containment has made single-use bioreactors possible. Mixing in single-use bioreactors can pose design problems. One disposable option is the Wave-style rocking platform bioreactor, a bag on a platform that oscillates back and forth to create waves in a solution contained within the bag; such systems are useful up to the about 100 L scale. Other options now allow single-use bioreactors to be scaled up to 2000 L (Scott, BioProcess International Supplement 5: 44-51, 2007). It is possible to use such single-use bioreactors for scale-up, while relying on the large, more permanent tanks for the final stages of the production process.

IX. Harvesting Cells, Extracts or Compounds from Tissue Culture

Methods of harvesting cultured cells, preparing extracts and purifying compounds from the in vitro cultures described herein are convention. The following provides representative but non-limiting techniques; one of ordinary skill in the art will appreciate the many additional techniques that are applicable.

Retrieval from a bioreactor can be carried out using convention means, for instance by siphoning off medium containing secreted metabolites and/or cell excretions—for instance as old medium is replaced or run through the system. Alternatively, the bioreactor can be drained of medium and biomass collected. One can also collect a portion (say, half or two thirds) of the biomass and restart the bioreactor run in those instances where the cells retain proliferation capabilities post initial run.

To extract intracellular components or metabolites, biomass is sonication or otherwise disrupted in a sterile liquid, such as water (with or without buffer(s)), alcohol, or another organic or inorganic solvent. Biomass can also be flash frozen in liquid nitrogen, followed by pulverization using: a homogenizer, such as a Dounce homogenizer, a motorized tissue homogenizer, a mortar and pestle, or alternate automated/mechanical grinding process. Also contemplated is freeze drying the biomass or drying the biomass in an oven or incubator followed by pulverization. Fresh biomass can also be ground using any of the previously listed methods and suspending in sterile alcohol, water (buffered or unbuffered), DMSO, or an organic or inorganic solvent.

Biofractionation and HPLC analysis can be carried out using art-recognized technologies. In fact, such methods can be carried out through a third party contractor, such as Chromadex (Irvine, Calif.). Commercial kits are also available, such as ApoAlert Cell Fractionation Kit (Clontech, Mountain View Calif.). Usually the manufacturer's protocols are followed when a commercially available kit is employed.

Optionally, cells are sub-fractionated before they are disrupted—for instance, if the component of interest is concentrated in a subcellular compartment (e.g., the vacuole), or in those embodiments where other cellular component(s) that are compartmentalized (e.g., hydrolases and the like) might be detrimental to the metabolite of interest. Methods for isolating specific sub-fractions of cells, including plant cells, are well known.

The following are representative (non-limiting) compositions of possible extraction buffers and solutions:
20 mM HEPES, pH 7.9, 10 mM KCl, 0.1 mM EDTA, 1.5 mM μM phenylmethylsulfonyl fluoride

(PMSF), 1 mM vanadate, 40 μg/ml leupeptin, and 1 μM MgCl2, 300 microcystin
20 mM HEPES, pH 7.9, 420 mM NaCl, 0.1 mM EDTA, 1.5 mM MgCl₂, and 25% glycerol containing 300 μM PMSF, 1 mM vanadate, 40 μg/ml leupeptin, 1 μM microcystin, and 150 U DNase 1
5 M urea, 2 M thiourea, 2% CHAPS, 2% SB-3, 40 mM Tris, pH 7.5, 10% glycerol, 150 IU/ml aprotinin, 2 μg/ml leupeptin, and 1 mM PMSF
Buffers provided with a commercial kit, such as ApoAlert Cell Fractionation Kit (Clontech, Mountain View Calif.) (e.g., the 1× Cell Fractionation Buffer)
RIPA Buffer Tris-HCl: 50 mM, pH 7.4·NP-40: 1%·Na-deoxycholate: 0.25%·NaCl: 150 mM·EDTA: 1 mM·PMSF: 1 mM·Aprotinin, leupeptin, pepstatin: 1 μg/ml each·Na3VO4: 1 mM·NaF: 1 mM

Also contemplated are systems wherein the desired metabolite is dissolved in the medium, for instance through secretion from the cells. Ways to extract media dissolved components are also convention, and include filtering the cell mass, or aspirating the medium, followed by biofractionation and HPLC selection and amplification of active components. As above, such analyses can be carried out using a third party contractor. Likewise, the supernatant/medium can be subject to fractionation using commercial kits. The medium can be spun down under vacuum, to yield a dry pellet of the secreted metabolites/proteins—which can then be resuspended and/or subjected to additional analyses. Analysis of the medium directly is also contemplated (e.g., run aliquots of media on gel electrophoresis to select individual proteins for further analysis or evaluation).

Also contemplated is supercritical fluid extraction (SEE, or Sat). Supercritical fluids are highly compressed gases that combine properties of gases and liquids. Supercritical fluids (e.g., supercritical fluid carbon dioxide) can be used to extract compounds, such as lipophilic or volatile compounds, from samples. Supercritical fluids are inexpensive, contaminant free, less costly to dispose of safely than organic solvents, and have solvating powers similar to organic solvents, but with higher diffusivities, lower viscosity, and lower surface tension. The solvating power can be adjusted by changing the pressure or temperature of the extraction process, or by adding modifiers to the supercritical fluid.

A typical supercritical fluid extractor consists of a tank of the mobile phase, such CO₂, a pump to pressurize the gas, an oven containing the extraction vessel, a restrictor to maintain a high pressure in the extraction line, and a trapping vessel. Analytes are trapped by letting the solute-containing supercritical fluid decompress into an empty vessel, through a solvent, or onto a solid sorbent material.

Examples of extraction systems are dynamic, static, or combination modes. In a dynamic extraction system, the supercritical fluid continuously flows through the sample in the extraction vessel and out the restrictor to the trapping vessel. In static system, the supercritical fluid circulates in a loop containing the extraction vessel for some period of time before being released through the restrictor to the trapping vessel. In a combination system, a static extraction is performed for some period of time, followed by a dynamic extraction.

The use of supercritical fluid extraction to obtain natural compounds and complexes is well known in the art. See, for instance, Natural Extracts Using Supercritical Carbon Dioxide, by Mamata Mukhopadhyay (CRC Press LLC, Boca Raton, Fla., 2000, ISBN 0-8493-0819-4). See also Extraction of Natural Products using Near-Critical Solvents, by King & Bott (Blackie Academic & Professional, 1993, ISBN 0 7514 0069 6).

For a description of additional extraction techniques, see Modern Extraction Techniques (Food and Agricultural Samples) by Turner (American Chemical Society, 2006, ISBN 9780841239401). Additional methods are referenced in the following section.

The method of extraction (and subsequent analysis) is often influenced by the type of metabolite or bio-active product that is the target of the project. There are myriad art-recognized systems for extraction, purification and analysis of plant (and other organism) metabolites, which can be exploited in this context. Thus, it is beneficial to discuss potential metabolites/products that are contemplated with regards to production in the described methods. The following is a non-limiting list of compounds: classic small “small molecules”, including Alkaloids (usually a small, heavily derivatized amino acid) (Hyoscyamine, present in Datura stramonium; Atropine, present in Atropa belladonna, Deadly nightshade; Cocaine, present in Erythroxylon coca the Coca plant; Codeine and Morphine, present in Papaver somniferum, the opium poppy; Tetrodotoxin, a microbial product in Fugu and some salamanders; Vincristine & Vinblastine, mitotic inhibitors found in the Rosy Periwinkle; nicotine, cocaine, and theobromine are also alkaloids), including Terpenoids (oligomerized semiterpenes) (Azadirachtin, Neem tree; Artemisinin, present in Artemisia annua Chinese wormwood; tetrahydrocannabinol, present in Cannabis sativa); Steroids (terpenes having a particular ring structure) and Saponins (plant steroids, often glycosylated); Glycosides (heavily modified sugar molecules) (such as Nojirimycin and Glucosinolates such as sinigrin); Phenols (such as Resveratrol and related compounds); Phenazines (including Pyocyanin, Phenazine-1-carboxylic acid, and related compounds or derivatives); bigger “small molecules” including Polyketides (e.g., Erythromycin, Discodermolide); Fatty acid synthase products (such as phloroglucinols and fatty acids and their derivative); Nonribosomal peptides (e.g., Vancomycin, Thiostrepton, Ramoplanin, Teicoplanin, Gramicidin, and Bacitracin); and compounds that bridge these categories (such as Epothilone). Also contemplated are polysaccharides and other biopolymers. The following table lists additional representative classes of metabolites:

| | | | |
|---|---|---|---|
| | Example | Example | |
| Class | Compounds | Sources | Some Effects and Uses |

NITROGEN-CONTAINING

| | | | |
|---|---|---|---|
| Alkaloids | nicotine cocaine | tobacco coca plant | interfere with neurotransmission, |
| | theobromine | chocolate (cacao) | block enzyme action |

NITROGEN-AND SULFUR-CONTAINING

| | | |
|---|---|---|
| Glucosinolates | sinigrin | cabbage, relatives |

TERPENOIDS

| | | | |
|---|---|---|---|
| Monoterpenes | menthol linalool | mint and relatives, | interfere with neurotransmission, |
| | | many plants | block ion transport, anesthetic |
| Sesquiterpenes | parthenolid | Parthenium and | contact dermatitis |
| | | relatives (Asteraceae) | |
| Diterpenes | gossypol | cotton | block phosphorylation; toxic |
| Triterpenes, cardiac | digitogenin | Digitalis (foxglove) | stimulate heart muscle, alter ion |
| glycosides | | | transport |
| Tetraterpenoids | carotene | many plants | antioxidant; orange coloring |
| Terpene polymers | rubber | Hevea (rubber) trees, | gum up insects; airplane tires |
| | | dandelion | |
| Sterols | spinasterol | spinach | interfere with animal hormone action |

PHENOLICS

| | | | |
|---|---|---|---|
| Phenolic acids | caffeic, chlorogenic | all plants | cause oxidative damage, browning in |
| | | | fruits and wine |
| Coumarins | umbelliferone | carrots, parsnip | cross-link DNA, block cell division |
| Lignans | podophyllin | mayapple, poison ivy | cathartic, vomiting, allergic |
| | urushiol | | dermatitis |
| Flavonoids | anthocyanin, | almost all plants | flower, leaf color; inhibit enzymes, |
| | catechin | | anti- and pro-oxidants, estrogenic |
| Tannins | gallotannin, | oak, hemlock trees, | bind to proteins, enzymes, block |
| | condensed tannin | birdsfoot trefoil, | digestion, antioxidants |
| | | legumes | |
| Lignin | lignin | all land plants | structure, toughness, fiber |

In some embodiments, the metabolite of interest is an anthocyanin or related pigment-type compound. Anthocyanins are recovered or extracted from cell cultures prepared by the methods described herein in ways similar to the methods known in the art for extraction of any other anthocyanins. For example, in cultured cells are homogenized and extracted with acidified water (0.1% sulfuric acid, pH 3.0). Modifications to the solvent used for extraction include the addition of ethanol or methanol (up to 50% volume/volume) and the use of acetic acid or any other food grade acid to acidify the solvent (instead of sulfuric acid). The cells may be frozen prior to homogenization if storage is required. For example, cells can be frozen in liquid nitrogen and stored at −80° C.

In one example, the cell suspension cultures are homogenized before removing the spent medium, and the resultant homogenate is filtered. The filtered homogenized cell mass can then be extracted with solvent to remove anthocyanins (or other pigments). In another example, the cell culture is filtered to remove the spent medium and solvent added to the remaining cell mass, then the cells are homogenized in the presence of solvent. In an additional example, spent medium is decanted, the solvent is added to the remaining cell mass, cells are homogenized, and anthocyanins extracted.

In all of the aforementioned examples, metabolite extraction with solvent may be repeated several times to extract as much of the metabolite as possible from the cell mass.

### X. Analysis of Extracts and Isolated or Purified Compounds

Extracts and components thereof that are prepared using methods described herein can be analyzed using convention biochemical, chemical, and biological systems.

Analytical chemistry techniques can be used to determine what is in an extract—and to identify and quantify known molecules. Example analytical chemistry analyses include: physical separation systems (e.g., liquid chromatography, high performance liquid chromatography, thin layer chromatography, electrophoresis, and so forth), mass separation systems (mass spectrometry, in all of its various embodiments), crystallography, thermal analysis, electrochemical analysis, microscopy, and combinations of two or more (so-called hybrid or combined technologies). Commercial companies are available for contacted analysis.

In addition to such physical analysis, the biological characterization of extracts and components are also contemplated. Such analyses involve evaluating the effect (if any) of a substance on a biological system, such as a cell, cell line, pool of cell lines, microbe, animal, model system, tissue or organ, or human subject. Particularly contemplated are monolayer cultures, reconstructed skin in vitro, insect and worms and other recognized animal models, as well as pre-clinical and clinical tests on human subjects and tissue samples.

Appropriate methods of analysis (and of extraction) are often influenced by the type of metabolite or bio-active product that is the target of the project. There are myriad art-recognized systems for extraction, purification and analysis of plant (and other organism) metabolites, which can be exploited in this context. The following is a non-limiting list of such references describing examples of such techniques: PCT publications WO 2008/074155; WO 99/035917; U.S. Pat. Nos. 7,208,181; 7,611,738; 6,238,673; 6,746,695; 7,582,674; 6,511,683; 7,396,554; U.S. patent publications no. 2007/0014912; 2009/0208544; Japan patent publication no. 2008/206479; and Gorgetti et al. (AAPS PharmSci 5(2):Article 20, 2003) and. Other references are cited herein.

Methods of measuring the amount or quantity of a pigment-like molecule, such as an anthocyanin, in a preparation are known to one of skill in the art. Anthocyanin content of a preparation (such as an extract of a cell culture) can be tested for its absorbance at 520 nm and anthocyanin content calculated using Beer's law (A520 nm×1000×MW of cyanidin glucoside)/(extinction coefficient).

The amounts and types of anthocyanins found in a preparation can be determined by LC-MS and by UV absorbance. In some examples, a preparation is injected to LC-MS analysis. The samples are monitored at 520 nm. Total anthocyanin concentration of an unknown extract can then be expressed as cyanidin-3,5-diglucoside equivalents by summing the peak areas at 520 nm and comparing to a standard curve.

Also provided herein are methods of assessing the biological effects of compounds and extracts generated from tissue cultures, which methods involve contacting the compounds (e.g., metabolites) or extracts with cells, then assessing alterations in gene expression in those cells in comparison to control cells not contacted with the compound or extract. Assessing such gene alterations can be carried out using convention methods, including for instance microarray analysis of gene expression changes. Collections of genes that have been found to be influenced by antioxidant(s), and/or that are now recognized as being involved in lifespan extension, cell longevity or health, mitochondrial biogenesis or function, telomere maintenance or DNA fidelity or repair, and so forth, such as those described in U.S. application Ser. No. 12/629,040 (filed Dec. 1, 2008 and incorporated herein in its entirety; published as US-2010-0173024 on Jul. 8, 2010) are particularly useful for assessing the biological function of compounds or extracts made by the methods describe herein. As described in that prior application, the identification of sets of genes that are responsive to antioxidant treatment and that act in a concerted manner (e.g., in a recognized pathway, in a similar manner as to magnitude and/or direction of change in gene expression, etc.) enables the production of tailored arrays that can be useful in characterizing the activities of known antioxidants, studying and identifying potential new antioxidant compositions, tracking the biological effect (e.g., on an experimental system or a subject) of an antioxidant treatment regimen, and analysis of, e.g., skin biopsy, blood, and other various body components.

Thus, it is particularly contemplated that the biological activity(s) of extracts and compositions derived from tissue cultures, such as those described in the Examples herein, can be examined and characterized by determining expression changes of genes found on the two custom arrays below. The genes in the first custom microarray (“Array 1”) were selected based on an exhaustive literature search for previously recognized longevity genes and lifespan altering genes. The second microarray (Array 2) includes the genes from the first array, plus select genes related to mitochondrial biogenesis, respiration efficiency, telomere maintenance, and genes. These customized arrays permit focused genetic analysis that is significantly faster than analyzing the entire human genome.

### Array 1 (Gene Symbols)

ACE
ACTB
APOE
BAX
BCL2
CASP2
CASP9
CCL4L1
CLK1
COX1
CREBBP
CYP19A1
DDC
GAPDH
GH1
HIGX1A
HLA-DRA
HPRT1
HSPA1A
HSPA1B
HSPA1L
IFI44
IGF1
IGF2
IL10
IL1A
IL6
KRAS
MAPK14
NADSYN1
NFKB1
NOS2A
PARP1
PARP2
PPARG
PTGS2
SHC1
SIRT1
SOD1
SOD2
TEP1
TERT
TNF
TP53

### Example Custom Microarray (Array 2)

| | | | | RT2 |
|---|---|---|---|---|
| Gene | | Refseq # | | Catalog |
| Symbol | Alias(es) | (GenBank) | Official Full Name | Number |
| PARP1 | ADPRT/ADPRT1/PARP/PARP-1/ | NM_001618 | Poly (ADP-ribose) | PPH00686 |
| | PPOL/pADPRT-1 | | polymerase 1 | |
| IGF1 | IGF1A/IGFI | NM_000618 | Insulin-like growth factor 1 | PPH00167 |
| | | | (somatomedin C) | |
| SHC1 | FLJ26504/SHC/SHCA | NM_003029 | SHC (Src homology 2 domain | PPH00229 |
| | | | containing) transforming | |
| | | | protein 1 | |
| IL1A | IL-1A/IL1/IL1-ALPHA/IL1F1 | NM_000575 | Interleukin 1, alpha | PPH00690 |
| NADSYN1 | FLJ10631/FLJ36703/FLJ40627 | NM_018161 | NAD synthetase 1 | PPH20527 |
| PARP2 | ADPRT2/ADPRTL2/ADPRTL3/ | NM_005484 | Poly (ADP-ribose) | PPH02684 |
| | PARP-2/pADPRT-2 | | polymerase 2 | |
| IGF2 | C11orf43/FLJ22066/FLJ44734/ | NM_000612 | Insulin-like growth factor 2 | PPH00168 |
| | INSIGF/pp9974 | | (somatomedin A) | |
| IFI44 | MTAP44/p44 | NM_006417 | Interferon-induced protein 44 | PPH01331 |
| IL6 | BSF2/HGF/HSF/IFNB2/IL-6 | NM_000600 | Interleukin 6 (interferon, beta 2) | PPH00560 |
| BAX | BCL2L4 | NM_004324 | BCL2-associated X protein | PPH00078 |
| PPARG | CIMT1/NR1C3/PPARG1/PPARG2/ | NM_015869 | Peroxisome proliferator- | PPH02291 |
| | PPARgamma | | activated receptor gamma | |
| CLK1 | CLK/CLK/STY/STY | NM_004071 | CDC-like kinase 1 | PPH02488 |
| CREBBP | CBP/KAT3A/RSTS | NM_004380 | CREB binding protein | PPH00324 |
| IL10 | CSIF/IL-10/IL10A/MGC126450/ | NM_000572 | Interleukin 10 | PPH00572 |
| | MGC126451/TGIF | | | |
| COX1 | MTCO1 | NP_536845 | Cytochrome c oxidase I | PPH60272 |
| APOE | AD2/LPG/MGC1571 | NM_000041 | Apolipoprotein E | PPH01366 |
| TERT | EST2/TCS1/TP2/TRT/hEST2 | NM_198255 | Telomerase reverse | |
| | | | transcriptase | |
| CYP19A1 | ARO/ARO1/CPV1/CYAR/CYP19/ | NM_000103 | Cytochrome P450, family 19, | PPH00132 |
| | MGC104309/P-450AROM | | subfamily A, polypeptide 1 | |
| TNF | DIF/TNF- | NM_000594 | Tumor necrosis factor (TNF | PPH00341 |
| | alpha/TNFA/TNFSF2 | | superfamily, member 2) | |
| PTGS2 | COX-2/COX2/GRIPGHS/PGG/HS/ | NM_000963 | Prostaglandin-endoperoxide | PPH01136 |
| | PGHS-2/PHS-2/hCox-2 | | synthase 2 (prostaglandin G/H | |
| | | | synthase and cyclooxygenase) | |
| HLA-DRA | HLA-DRA1 | NM_019111 | Major histocompatibility | PPH00857 |
| | | | complex, class II, DR alpha | |
| TEP1 | TLP1/TP1/TROVE1/VAULT2/ | NM_007110 | Telomerase-associated protein | PPH02434 |
| | p240 | | 1 | |
| BCL2 | Bcl-2 | NM_000633 | B-cell CLL/lymphoma 2 | PPH00079 |
| HSPA1A | FLJ54303/FLJ54370/FLJ54392/ | NM_005345 | Heat shock 70 kDa protein 1A | PPH01193 |
| | FLJ54408/FLJ75127/HSP70-1/ | | | |
| | HSP70-1A/HSP70I/HSP72/HSPA1/ | | | |
| | HSPA1B | | | |
| HSPA1B | FLJ54328/HSP70-1B/HSP70-2/ | NM_005346 | Heat shock 70 kDa protein 1B | PPH01216 |
| | HSPA1A | | | |
| DDC | AADC | NM_000790 | Dopa decarboxylase (aromatic | PPH19374 |
| | | | L-amino acid decarboxylase) | |
| SIRT1 | SIR2L1 | NM_012238 | Sirtuin (silent mating type | PPH02188 |
| | | | information regulation 2 | |
| | | | homolog) 1 (S. cerevisiae) | |
| KRAS | C-K-RAS/K-RAS2A/K-RAS2B/ | NM_004985 | V-Ki-ras2 Kirsten rat sarcoma | PPH00181 |
| | K-RAS4A/K-RAS4B/KI-RAS/ | | viral oncogene homolog | |
| | KRAS1/KRAS2/NS3/RASK2 | | | |
| SOD1 | ALS/ALS1/IPOA/SOD/ | NM_000454 | Superoxide dismutase 1, | PPH00234 |
| | homodimer | | soluble | |
| HSPA1L | HSP70-1L/HSP70-HOM/ | NM_005527 | Heat shock 70 kDa protein 1- | PPH01206 |
| | HSP70T/hum70t | | like | |
| ACE | ACE1/CD143/DCP/DCP1/ | NM_000789 | Angiotensin I converting | PPH02581 |
| | MGC26566/MVCD3 | | enzyme (peptidyl-dipeptidase | |
| | | | A) 1 | |
| TP53 | FLJ92943/LFS1/TRP53/p53 | NM_000546 | Tumor protein p53 | PPH00213 |
| SOD2 | IPO-B/MNSOD/Mn-SOD | NM_000636 | Superoxide dismutase 2, | PPH01716 |
| | | | mitochondrial | |
| CASP9 | APAF-3/APAF3/CASPASE-9c/ | NM_001229 | Caspase 9, apoptosis-related | PPH00353 |
| | ICE-LAP6/MCH6 | | cysteine peptidase | |
| CCL4L1 | AT744.2/CCL4L/LAG-1/ | NM_001001435 | Chemokine (C-C motif) | PPH23119 |
| | LAG1/SCYA4L | | ligand 4-like 1 | |
| GH1 | GH/GH-N/GHN/hGH-N | NM_000515 | Growth hormone 1 | PPH00577 |
| MAPK14 | CSBP1/CSBP2/CSPB1/EXIP/ | NM_001315 | Mitogen-activated protein | PPH00750 |
| | Mxi2/PRKM14/PRKM15/RK/ | | kinase 14 | |
| | SAPK2A/p38/p38ALPHA | | | |
| NOS2 | HEP-NOS/INOS/NOS/NOS2A | NM_000625 | Nitric oxide synthase 2, | PPH00173 |
| | | | inducible | |
| CASP2 | CASP-2/ICH-1L/ICH-1L/ | NM_032982 | Caspase 2, apoptosis-related | PPH00111 |
| | 1S/ICH1/NEDD2 | | cysteine peptidase | |
| NFKB1 | DKFZp686C01211/EBP-1/ | NM_003998 | Nuclear factor of kappa light | PPH00204 |
| | KBF1/MGC54151/NF-kappa-B/ | | polypeptide gene enhancer in | |
| | NFKB-p105/NFKB-p50/ | | B-cells 1 | |
| | p105/p50 | | | |
| NOS1 | IHPS1/NOS/nNOS | NM_000620 | Nitric oxide synthase 1 | PPH01304 |
| | | | (neuronal) | |
| NOS3 | ECNOS/eNOS | NM_000603 | Nitric oxide synthase 3 | PPH01298 |
| | | | (endothelial cell) | |
| IL8 | CXCL8/GCP-1/GCP1/ | NM_000584 | Interleukin 8 | PPH00568 |
| | LECT/LUCT/LYNAP/ | | | |
| | MDNCF/MONAP/NAF/ | | | |
| | NAP-1/NAP1 | | | |
| IL11 | AGIF/IL-11 | NM_000641 | Interleukin 11 | PPH00573 |
| IL33 | C9orf26/DKFZp586H0523/DVS27/ | NM_033439 | Interleukin 33 | PPH17375 |
| | NF-HEV/NFEHEV/RP11-575C20.2 | | | |
| VEGFA | MGC70609/MVCD1/VEGF/ | NM_003376 | Vascular endothelial growth | PPH00251 |
| | VEGF-A/VPF | | factor A | |
| FOS | AP-1/C-FOS | NM_005252 | V-fos FBJ murine | PPH00094 |
| | | | osteosarcoma viral oncogene | |
| | | | homolog | |
| JUN | AP-1/AP1/c-Jun | NM_002228 | Jun oncogene | PPH00095 |
| MMP1 | CLG/CLGN | NM_002421 | Matrix metallopeptidase 1 | PPH00120 |
| | | | (interstitial collagenase) | |
| TIMP3 | HSMRK222/K222/K222TA2/ | NM_000362 | TIMP metallopeptidase | PPH00762 |
| | SFD | | inhibitor 3 | |
| COL1A1 | OI4 | NM_000088 | Collagen, type I, alpha 1 | PPH01299 |
| EGF | HOMG4/URG | NM_001963 | Epidermal growth factor | PPH00137 |
| | | | (beta-urogastrone) | |
| EGR2 | AT591/CMT1D/CMT4E/ | NM_000399 | Early growth response 2 | PPH01478 |
| | DKFZp686J1957/FLJ14547/ | | | |
| | KROX20 | | | |
| PDGFRL | PDGRL/PRLTS | NM_006207 | Platelet-derived growth factor | PPH13429 |
| | | | receptor-like | |
| TGFB1 | CED/DPD1/TGFB/TGFbeta | NM_000660 | Transforming growth factor, | PPH00508 |
| | | | beta 1 | |
| PARP3 | ADPRT3/ADPRTL2/ADPRTL3/ | NM_005485 | Poly (ADP-ribose) | PPH02698 |
| | IRT1/PADPRT-3 | | polymerase family, member 3 | |
| PARP4 | ADPRTL1/PARPL/PH5P/VAULT3/ | NM_006437 | Poly (ADP-ribose) | PPH20757 |
| | VPARP/VWA5C/p193 | | polymerase family, member 4 | |
| TPP1 | CLN2/GIG1/LPIC/MGC21297 | NM_000391 | Tripeptidyl peptidase I | PPH20033 |
| POT1 | DKFZp586D211/hPot1 | NM_015450 | POT1 protection of telomeres | PPH08949 |
| | | | 1 homolog (S. pombe) | |
| RAP1A | KREV-1/KREV1/RAP1/SMGP21 | NM_002884 | RAP1A, member of RAS | PPH02284 |
| | | | oncogene family | |
| TERF2 | TRBF2/TRF2 | NM_005652 | Telomeric repeat binding | PPH02738 |
| | | | factor 2 | |
| TINF2 | TIN2/TIN2L | NM_012461 | TERF1 (TRF1)-interacting | PPH02468 |
| | | | nuclear factor 2 | |
| GPX1 | GSHPX1/MGC14399/MGC88245 | NM_000581 | Glutathione peroxidase 1 | PPH00154 |
| SIRT2 | SIR2/SIR2L/SIR2L2 | NM_012237 | Sirtuin (silent mating type | PPH18336 |
| | | | information regulation 2 | |
| | | | homolog) 2 (S. cerevisiae) | |
| SIRT4 | MGC130046/MGC130047/ | NM_012240 | Sirtuin (silent mating type | PPH09423 |
| | MGC57437/SIR2L4 | | information regulation 2 | |
| | | | homolog) 4 (S. cerevisiae) | |
| KL | — | NM_004795 | Klotho | PPH13489 |
| PPARGC1A | LEM6/PGC-1(alpha)/PGC-1v/ | NM_013261 | Peroxisome proliferator- | PPH00461 |
| | PGC1/PGC1A/PPARGC1 | | activated receptor gamma, | |
| | | | coactivator 1 alpha | |
| HSPA6 | — | NM_002155 | Heat shock 70 kDa protein 6 | PPH01192 |
| | | | (HSP70B′) | |
| BCL2L1 | BCL-XL/S/BCL2L/BCLX/Bcl-X/ | NM_138578 | BCL2-like 1 | PPH00082 |
| | DKFZp781P2092/bcl-xL/ | | | |
| | bcl-xS | | | |
| FOXO3 | AF6q21/DKFZp781A0677/FKHRL1/ | NM_001455 | Forkhead box O3 | PPH00807 |
| | FKHRL1P2/FOXO2/FOXO3A/ | | | |
| | MGC12739/MGC31925 | | | |
| HMOX1 | HO-1/HSP32/bK286B10 | NM_002133 | Heme oxygenase (decycling) | PPH00161 |
| | | | 1 | |
| TIMM22 | TEX4/TIM22 | NM_013337 | Translocase of inner | PPH10377 |
| | | | mitochondrial membrane 22 | |
| | | | homolog (yeast) | |
| TOMM40 | C19orf1/D19S1177E/PER-EC1/ | NM_006114 | Translocase of outer | PPH16749 |
| | PEREC1/TOM40 | | mitochondrial membrane 40 | |
| | | | homolog (yeast) | |
| SERPINB2 | HsT1201/PAI/PAI-2/ | NM_002575 | Serpin peptidase inhibitor, | PPH00793 |
| | PAI2/PLANH2 | | clade B (ovalbumin), member 2 | |
| KIT | C-Kit/CD117/PBT/SCFR | NM_000222 | V-kit Hardy-Zuckerman 4 | PPH00432 |
| | | | feline sarcoma viral oncogene | |
| | | | homolog | |
| NEIL1 | FLJ22402/FPG1/NEI1/hFPG1 | NM_024608 | Nei endonuclease VIII-like 1 | PPH02702 |
| | | | (E. coli) | |
| CRP | MGC149895/MGC88244/PTX1 | NM_000567 | C-reactive protein, pentraxin- | PPH02632 |
| | | | related | |
| DUSP2 | PAC-1/PAC1 | NM_004418 | Dual specificity phosphatase 2 | PPH00049 |
| IMMP1L | FLJ25059/IMP1/IMP1-LIKE | NM_144981 | IMP1 inner mitochondrial | PPH17716 |
| | | | membrane peptidase-like (S. | |
| | | | cerevisiae) | |
| HBEGF | DTR/DTS/DTSF/HEGFL | NM_001945 | Heparin-binding EGF-like | PPH02589 |
| | | | growth factor | |
| SIRT3 | SIR2L3 | NM_012239 | Sirtuin (silent mating type | PPH22989 |
| | | | information regulation 2 | |
| | | | homolog) 3 (S. cerevisiae) | |
| CDKN2A | ARF/CDK4I/CDKN2/CMM2/INK4/ | NM_000077 | Cyclin-dependent kinase | PPH00207 |
| | INK4a/MLM/MTS1/TP16/ | | inhibitor 2A (melanoma, p16, | |
| | p14/p14ARF/p16/p16INK4/ | | inhibits CDK4) | |
| | p16INK4a/p19 | | | |
| BMP2 | BMP2A | NM_001200 | Bone morphogenetic protein 2 | PPH00549 |
| COL3A1 | EDS4A/FLJ34534 | NM_000090 | Collagen, type III, alpha 1 | PPH00439 |
| UBE2S | E2-EPF/E2EPF/EPF5 | NM_014501 | Ubiquitin-conjugating enzyme | PPH02166 |
| | | | E2S | |
| GCH1 | DYT14/DYT5/GCH/ | NM_000161 | GTP cyclohydrolase 1 | PPH05828 |
| | GTP-CH-1/GTPCH1 | | | |
| PARP9 | BAL/BAL1/DKFZp666B0810/ | NM_031458 | Poly (ADP-ribose) | PPH12091 |
| | DKFZp686M15238/FLJ26637/ | | polymerase family, member 9 | |
| | FLJ35310/FLJ41418/FLJ43593/ | | | |
| | MGC: 7868 | | | |
| S100A7 | PSOR1/S100A7c | NM_002963 | S100 calcium binding protein A7 | PPH11216 |
| GAPDH | G3PD/GAPD/MGC88685 | NM_002046 | Glyceraldehyde-3-phosphate | PPH00150 |
| | | | dehydrogenase | |
| ACTB | PS1TP5BP1 | NM_001101 | Actin, beta | PPH00073 |
| HPRT1 | HGPRT/HPRT | NM_000194 | Hypoxanthine | PPH01018 |
| | | | phosphoribosyltransferase 1 | |

### XI. Detection and Quantification of DNA Damage

Another way to assess the biological activity of a metabolite preparation produced using the cultures and methods described herein is to determine whether and to what extent it inhibits DNA damage—that is, to what extent it can protect DNA from oxidative and other damage. This can be assessed by measuring DNA damage in cells with and without treatment with text agent (metabolite, extract, etc.), or with varying amounts or under varying other conditions.

DNA damage, including that caused by oxidation, can be measured by any art known technique. Methods for assessing DNA damage are well known; see, for instance, Loft & Poulsen (Free Radic. Res. 33:S67-83, 2000). By way of example, the level of oxidative DNA damage in an organ or cell may be studied by measurement of modified bases in extracted DNA by immunohistochemical visualization, and from assays of strand breakage before and after treatment. Oxidatively modified nucleobases can be measured in the DNA and strand breaks can be detected by the comet assay, optionally with the use of repair enzymes introducing breaks at oxidized bases. Oxidized bases and nucleosides from DNA repair, the nucleotide pool and cell turnover can be measured in urine. The excretion rate represents the average rate of damage in the body, whereas the level of oxidized bases in DNA is a concentration measurement in the specific cells.

The comet assay, also called the ‘Single Cell Gel Assay’, is a well known technique to detect DNA damage and repair at the level of single cells. This technique was developed by Swedish researchers Östling & Johansson (Biochem. Biophys. Res. Commun. 123:291-298, 1984), who demonstrated that DNA in one or a few cells embedded in low-melt agarose migrates out of the cell in an electrophoretic field in a pattern that is influenced by the extent of the DNA damage. The comet assay was later modified by Singh et al. (Exp. Cell Res., 175:184-191, 1988), and is now described as the alkaline comet assay. The comet assay is one of the most popular tests of DNA damage (e.g., single- and double-strand breaks, oxidative-induced base damage, and DNA-DNA/DNA-protein cross linking) detection by electrophoresis that has been developed. The assay is described and reviewed in the following references: McKelvey-Martin et al., Mutat. Res. 288: 47-63, 1993; Fairbairn et al., Mutat. Res. 339: 37-59, 1995; Anderson et al., Mutagenesis 13: 539-555, 1998; Rojas et al., J. Chromat. B Biomed Sci Appl 722: 225-254, 1999; Tice et al., Environ Mol Mutagen 35(3):206-21, 2000; Collins, Methods Mol. Biol. 203:163-177, 2002; Olive, Methods Mol. Biol. 203:179-194, 2002; Faust et al., Mutat. Res. 566:209-229, 2004.

In addition, the comet assay can be adapted in order to detect oxidized pyrimidines and purines (such as 8-oxo-guanine) by digestion of the embedded nucleoid samples with endonuclease III and formamidopyrmidine glycosylase (FPG), respectively. The additional breaks formed at the site of base oxidations increase the relative amount of DNA in the tail of the resultant comet. See, for instance, Collins et al., Carcinogenesis 19:2159-2162, 1998.

8-Hydroxy-2′-deoxyguanosine (8-OHdG) is one of the most commonly used markers for assessing oxidative DNA damage. This compound is also sometimes referred to as 8-oxy-7-hydrodeoxyguanosine (8-oxodG). DNA can be oxidized to produce many oxidative products; however oxidation of the C-8 of guanine is one of the more common oxidative events, and results in a mutagenic lesion that produces predominantly G-to-T transversion mutations. 8-OHdG can be measured in DNA samples (such as lymphocyte DNA) and in urine (Wu et al., Clin. Chim. Acta. 39:1-9, 2004). Several methods for quantitating this biomarker are available. HPLC with electrochemical detection (HPLC/ECD) and GC/MS methods are widely used (see, e.g., Cadet et al., Free Radic. Biol. Med. 33:441-49, 2002; Cooke et al., Free Radic. Res. 32:381-397, 2000). Enzyme-linked immunosorbent assay (ELISA) techniques are also being employed (Santella, Canc. Epidemiol. Biomarkers Prev. 8:733-739, 1999).

Additional methods of assaying and/or quantifying oxidative damage to DNA are known to those of ordinary skill in the art. See, for instance, Cadet et al., Biol. Chem. 383:933-943, 2002; Kasai, Free Radic. Biol. Med. 33:450-456, 2002; and Halliwell, Am. J. Clin. Nutr. 72:1082-1087, 2000.

As used herein, a reduction in oxidative DNA damage is any measurable reduction in oxidized DNA in a subject, or any measurable reduction in a marker for oxidized DNA. Thus, for instance, a reduction in oxidation DNA damage can be measured as reduction in the size of comet observed, using a comet assay, or a reduction in the level of an oxidative DNA product (such as 8-OHdG) in a subject, compared to a time before administration of a metabolite composition, or in comparison to a subject not receiving the metabolite composition. In certain embodiments, the reduction is a reduction in the endogenous level of oxidative DNA damage.

By way of example, methods provided herein will result in at least a 10% reduction in oxidative DNA damage. In other embodiments, administration of the metabolite or metabolite-enriched extract results in at least a 15% reduction in oxidative DNA damage; at least 25% reduction, at least 30% reduction, at least 40% reduction, or more. In particularly beneficial embodiments, the level of endogenous oxidative DNA damage is reduced by at least 20% or more, for instance, at least 25%, at least 30%, at least 40%, at least 50%, at least 60%, at least 75%, at least 80%, or more. The reduction in oxidative DNA damage may be transient, and is expected to be linked to the dosage and time (duration) of administration of the metabolite or metabolite-enriched extract.

It is understood that a measured reduction in oxidative DNA damage may include outright prevention of the oxidative damage, reversal of damage that has already occurred, or a combination of these.

### XII. Methods of Use and Formulation of Compositions

The present disclosure includes treatment or supplements that alter or influence health, longevity, and/or lifespan (e.g., by inhibiting DNA damage, including oxidation damage, repairing DNA damage, inhibiting damage to mitochondria or mitochondrial respiration, increasing mitochondrial biogenesis, etc.) in a subject such as an animal, for example a rat or human. The method includes administering a metabolite (pure or in the form of an extract), or a combination of metabolite and one or more other pharmaceutical or nutritional agents, to the subject optionally in a pharmaceutically compatible carrier. The metabolite is administered in an effective amount to measurably reduce, prevent, inhibit, reverse or otherwise decrease oxidative DNA damage in a cell of the subject, or to increase mitochondria efficiency (e.g., respiration or respiration efficiency), mitochondria number, prolong cell division, increase metabolic state of cell, increasing transcription or translation rate and/or accuracy

Metabolite preparations and isolated and purified compounds generated by the methods disclosed herein can be administered to a subject for therapeutic, dietary, or cosmetic purposes, for instance. The subject can be a human or other mammal, such as a monkey, a horse, a cow, a pig, a dog, a cat, a mouse or a rat.

For instance, anthocyanins are extensively used as natural color additives in many food products such as soft drinks, beverages and yogurts. In those embodiments where the metabolite is or comprises an anthocyanin, the metabolite preparation may be used in any food, beverage, drug, cosmetic, or other preparation in place of conventionally prepared anthocyanins.

The treatment can be used prophylactically in any subject, since all subjects are exposed to aging and oxidative damage through metabolic processes, environmental exposure, and other influences. In addition, the treatment can be supplied to a subject in a demographic group at significant risk for particular oxidative damage. Subjects can also be selected using more specific criteria, such as a definitive diagnosis of a condition leaving the subject prone to the depredations of oxidative DNA damage. The administration of any exogenous metabolite would inhibit the progression of, and/or reverse, the oxidation associated disease as compared to a subject to whom the metabolite was not administered. The antioxidant effect, however, increases with the dose of metabolite.

The vehicle in which the metabolite is delivered can include pharmaceutically acceptable compositions of metabolite using methods well known to those with skill in the art. Any of the common carriers, such as sterile saline or glucose solution, can be utilized with the compositions provided herein. Routes of administration include but are not limited to oral, intracranial ventricular (icv), intrathecal (it), intravenous (iv), parenteral, rectal, topical ophthalmic, subconjunctival, nasal, aural, sub-lingual (under the tongue) and transdermal. The metabolite may be administered intravenously in any conventional medium for intravenous injection such as an aqueous saline medium, or in blood plasma medium. Such medium may also contain conventional pharmaceutical adjunct materials such as, for example, pharmaceutically acceptable salts to adjust the osmotic pressure, lipid carriers such as cyclodextrins, proteins such as serum albumin, hydrophilic agents such as methyl cellulose, detergents, buffers, preservatives and the like. For instance, U.S. Pat. No. 6,132,790 to Schlipalius describes methods of making water miscible compositions comprising carotenoid.

Metabolite(s) and/or crude extract comprising such can be used directly after being dissolved in ethanol and diluted with water. It can also be prepared into a latex preparation. A latex preparation can be prepared by adding gallic acid, L-ascorbic acid (or its ester or salt), gum (e.g., locust bean gum, qua gum or gelatin), vitamin P (e.g., flavoids such as hesperidin, lutin, quercetine, catechin, thianidine and eliodictin or mixtures thereof) to the aqueous phase, or by adding metabolite, metabolite crude extract or a mixture thereof to the oil phase, and then adding glycerine fatty acid ester or oil, examples of which include vegetable seed oil, soy bean oil, corn oil and other routinely used liquid oils. A high-speed agitator or homogenizer can be used to emulsify such compositions.

The compounds and extracts described herein or identified using the methods described herein can be provided in capsules and the like, for instance by suspending the metabolite in oil directly or by way of incorporation with an emulsifier. Alternatively, the metabolite product can be used in a powder, for instance, it can be spray dried and provided in the form of a liquid or powder. By way of example, U.S. Pat. Nos. 6,976,575 and 5,827,539, both to Gellenbeck, describe production of dry carotenoid-oil powders.

Esters are highly soluble in, and can be easily dissolved in, oils. Examples of such oils include vegetable oils such as soy bean oil, corn oil, rape seed oil, palm oil, olive oil, safflower oil, lemon oil, orange oil, peanut oil and sunflower oil, hardened oils produced by hydrogenating these oils, natural waxes such as lanolin, whale wax and bees wax, animal fats such as beef tallow, pork tallow and butter as well as wheat germ oil and concentrated vitamin E oil. In addition, glycerine fatty acid ester, sucrose fatty acid ester, sorbitan fatty acid ester, soy bean phospholipid, propylene glycol fatty acid ester and stearate diglyceride can be used as emulsifiers.

Embodiments of the disclosure comprising compositions, including food, cosmetic and pharmaceutical compositions, that can be prepared with optional conventional acceptable carriers, adjuvants and/or counterions as would be known to those of ordinary skill in the art. Suitable excipients include, e.g., organic and inorganic substances that are appropriate for enteral, parenteral, or oral administration, e.g., water, saline, buffers, vegetable oils, mineral oils, benzyl alcohol, cyclodextrin, hydroxypropylcyclodextrin (for instance, beta-hydroxypropylcyclodextrin), polyethylene glycols, glycerol triacetate and other fatty acid glycerides, gelatin, soya lecithin, carbohydrates such as lactose or starch or other sugars, magnesium stearate, talc or cellulose. The preparations can be sterilized and/or contain additives, such as preservatives or stabilizers. Metabolite(s) can be formulated with various oils, including coconut, sunflower, mustard, almond, sesame, safflower, or peanut.

For instance, for use in the provided methods and compositions, metabolite (in pure form or in the form of an extract) can be mixed for instance in an oil, then encapsulated in softgel capsules for oral ingestion. The oils can vary and in various embodiments include virtually any edible or consumable oil, particularly vegetable oils including but not limited to natural oils, such as omega-3 and omega-6 fatty acids found in the Haematococcus algae, rice bran oil, olive oil, cranberry seed oil, or mixtures of two or more thereof.

Other modes of encapsulations are contemplated, and will be known to the skilled artisan. Encapsulation may be of particular applicability for dermatologic purposes. Examples of encapsulation systems include microencapsulation, biopolymer microsponges, lipid liposomal encapsulation, time release degradable microparticles, and so forth. Biomaterials can also be enclosed in diatoms for a ‘natural’ nanoparticle or microparticle carrier.

The compositions in some embodiments are in the form of a unit dose in solid, semi-solid and liquid dosage forms such as tablets, pills (such as enteric-coated pills), capsules, powders, stabilized beadlets (which optionally are compressed into a tablet or other form), granules, suppositories, liquid solutions or suspensions, injectable and infusible solutions. Also contemplated are lotions, creams, and other topical application compositions.

Although the dose varies according to the purpose of administration and status of the patient (sex, age, body weight and so forth), the normal adult dose of metabolite in the case of oral administration is 0.1 mg (100 μg) to 10 g per day and preferably 0.1 mg (100 μg) to 1 g per day. The range for obtaining preventive effects is 0.01 mg (10 μg) to 100 mg per day, for instance about 0.1 mg (100 μg) to 10 mg per day. Specific example daily dosages include 500 μg, 1 mg, 2 mg, 3 mg, 4, mg, 6 mg, 8 mg, 10 mg, and so forth, for instance to be provided to an adult human.

Alternatively, dosages in some embodiments are applied in order to raise the plasma level of metabolite in the subject for a period of time, for instance, for a period of at least one week, or more. In various embodiments, dosages of metabolite(s) are administered to a subject to increase the plasma metabolite level to at least 0.05 μmol/L (μmolar, or μM). In other embodiments, the level is increased to at least 0.06 μM, at least 0.08 μM, at least 0.1 μM, at least 1.2 μM, at least 1.4 μM or more. In various embodiments, the level of metabolite is maintained for more than a week, for instance, for at least two weeks, at least a month, or longer. In some instances, it is beneficial to continue maintenance of the metabolite dosage, and therefore the level of metabolite in the subject's system, for periods measured in months or years. Optionally, control-dose infusion pumps or other like devices are employed to govern the dosage of the metabolite.

The preparations and methods described herein can be utilized in both human and veterinary medicine.

Metabolite preparations and isolated and purified compounds generated by the methods disclosed herein can be administered to a subject for therapeutic, dietary, or cosmetic purposes, for instance. The subject can be a human or other mammal, such as a monkey, a horse, a cow, a pig, a dog, a cat, a mouse or a rat.

In those embodiments where the metabolite is or comprises an anthocyanin, the metabolite preparation may be used in any food, beverage, drug, cosmetic, or other preparation in place of conventionally prepared anthocyanins.

Thus, in another aspect, the disclosure provides a food supplement or pharmaceutical composition, which composition comprises metabolite together with a food supplement or pharmaceutically accepted diluent or carrier. Veterinary applications are also contemplated, as animals also benefit from antioxidant and other health-increasing compounds.

In carrying out the methods provided herein, the metabolite may be used together with other active agents, such as, for example: another carotenoid (e.g., lycopene or alpha, beta, gamma or delta carotene), one or more other antioxidants (such as vitamin A, vitamin C, vitamin E (α-tocopherol and other active tocopherols)), selenium, copper, zinc, manganese and/or ubiquinone (coenzyme Q10). It is appreciated in the art that oral metabolite can be partially destroyed in the gastrointestinal tract, thereby lowering the effectively applied dosage. By providing vitamin E and/or vitamin C to the subject, this process in inhibited and more carotenoid is absorbed by the subject. The inhibitor may be included as part of a composition as part of a composition described herein, or administered separately.

The following examples are provided to illustrate certain particular features and/or embodiments. These examples should not be construed to limit the invention to the particular features or embodiments described.

### EXAMPLES

### Example 1

### Base Methodology for Production of Plant Tissue Culture

This example provides a general overview of guidelines for handling plant tissue for producing tissue culture. Specific methods are provided in later examples.

General Plant Cell Culture: Generally, the “youngest” original tissue is selected as juvenile tissue tends to be less contaminated and often readily forming callus tissue. For instance, for orchids, using protocorm tissue for generation of callus tissue may be the fastest route. Protocorms are beneficially used before they develop root or shoot, and the dark green protocorms should be selected (as clear or pale protocorms are generally unhealthy. An orbital shaker is useful to reduce any influence gravity might have on the cells, and low speed is generally recommended in order to keep to a minimum shearing and impact stress to the cells. For solid cultures, 45 degree slants are used; this permits maximum air exchange. If cultures need to be kept in the dark, they can be wrapped in sterile foil or keep in a drawer; it is important to ensure that the temperature is maintained (e.g., 72° C. or 68° C. degrees for berry). All charcoal should be removed from callus media, as it depletes the medium of auxins. Callus tissue in general is subcultured every week to 10 days (or in some instances, as long as 2 weeks). When subculturing, the tissue is rinsed in sterile H₂O and then placed on or in the fresh medium. Placing filter paper over liquid media permits sterilized (coffee) bean/seed germination. GelRite (Gellem Gum) has been observed to be superior to agar for solid media applications, and is generally used at 2% or 2.2-2.5% if the media is altered. Sterile procedures should be carefully observed at all times and with all procedures, particularly once new/fresh material has been sterilized. Plants, trees and soils are maintained with a disinfectant, such as Physan™ (1 teaspoon per gallon) and area surrounding material sanitized. It is advantageous to test varying ages of plant/tissue/seeds for best culturing or germination results

Specific Plants of LSE research (general information): Unless otherwise specified, green coffee cherry seeds are used for callus formation. It is easier to disinfect the outside of the coffee berry, specifically the harder green berry, using 10% bleach and 1-2 drops of Tween 20 for 10 minutes (shaken). For green tea, it is recommended to only use the apical meristem for callus formation/culture. Other parts of the tea plant need modifications to the media and/or procedures described herein, in order to optimize callus formation. For Blueberry/Strawberry, fruit are generally used to generate callus tissue. It is easier to sterilize firmer, less ripe berries.

Stage I media (used for generating a sterile in vitro culture and propagating plants) is basically M and S basal media (e.g., Phytechnology (Shawnee Mission, Kans., USA) catalog #M527 Murashige Modified Multiplication Basal Medium). This media may be refined dependent upon the results of the initial culture tests (callus formation, death, contamination etc. . . . ). Stage II media (used for generation of callus tissue or in vitro propagation) is Phytechnology (Shawnee Mission, Kans., USA) catalog #M401 Murashige and Skoog Modified Medium; it can be modified to make it stronger version to keep sugars and protocorm size up. Laboratory stocks of media are maintained, and hormones, vitamins, and other adjunctives are added only when needed (before use), to allow for greater control and flexibility for research purposes. NAA is useful for generating callus tissue (in relatively high doses); there should be no BA (or other cytokinin) present for optimal callus production. IAA is similar to NAA, but is generally a weaker strength auxin. Other hormones will be known to those of skill in the art.

For propagation via microcutting: Only active growing meristems/root/shoot tips are selected.

Woody sections of the plant are avoided, as they are generally too old to produce strong, viable cultures. Cut a larger section than needed (more area around the active growth) to protect tissue from bleaching agents and death. Place the excised tissue in a sterile tube with 10% bleach and 1 drop of Tween 20 for ten minutes with agitation. (Optionally, the duration of submersion can be increased—but it is then beneficial to decrease the concentration of bleach). It is generally better to do fewer cuttings at a time—no more than about five at a time is a good rule. (Optionally, cuttings can be wrapped in cheesecloth to prevent or reduce tissue injury to the plant cuttings and make it easier to rinse and keep in the tube.). Empty and rinse cuttings in tube with autoclaved H₂O (minimum 3× or until the suds are gone). Place the sterilized samples in sterile Petri dish, and use a sterile scalpel to cut sections at the base of the leaves or other tissue. Remove the leaves from those sections and place into multiplication media. The cut surfaces are usually fully submerged into the media (liquid or solid), to allow for absorption of nutrients. It is advantageous to experiment with factors (media, light, temp, etc. . . . ) until the maximum efficacy of callus formation is achieved. Guidelines for such variation are explored herein; also, this practice will be known to those of skill in the art. For microdivision/propagation, repeat this process (remove the apical and/or lateral meristems and culture) to generate new plant cultures. This can be repeated indefinitely. Generally, apical meristem is used for callus formation.

### Example 2

### Acai Palm (Euterpe oleracea) Seed Culture

Euterpe oleracea seed were prepped as follows for sterile culture: Seed (obtained from Brazil) was already germinated when received. Husk hairs were removed, then seeds were surface sterilized by putting 6 seeds in 3% hydrogen peroxide for 10 minutes. The seeds were not rinsed. Alternatively, the full (including furry seed coat) berry was treated in 50% bleach+Tween 20 for 20 minutes, or 10% bleach+Tween 20 for 60 minutes. The seed coat and any “shoots” were removed and the berry was treated a second time with 10% bleach+Tween 20 for 2 minutes. The berries were then placed into culture per basic methodology.

Tubes were filled with Stage 1 gel medium {M527 Phytotechnology: Murashige Modified Multiplication Basal Medium} and sterilized. Germinated seed was transferred to the slants (1 seed/tube). Tubes were capped and wrapped with parafilm. One seed was placed in each of 6 tubes. Tubes were labeled with the seed name, method of sterilization, and date of culturing. Tubes were stored at room temperature.

All acai seeds died or were contaminated after less than a week in culture.

### Example 3

### Alaska Blueberry (Vaccinium alaskensis) Seed Culture

Vaccinium alaskensis seed were prepped as follows for germination: Seed (obtained from Alaska Blues, LLC Calder Mt. Tongass Nat'l Rain Forest, Alaska) was surface sterilized in 3% hydrogen peroxide for 5 minutes. The hydrogen peroxide was removed by a vacuum pump and a filter sterilization unit. Seed was rinsed with sterile deionized water and vacuum pumped to dry. Alternatively, seed were treated with 5% bleach (Clorox) for 70 minutes, then rinsed.

Tubes were filled with Stage 1 gel medium {M527 Phytotechnology: Murashige Modified Multiplication Basal Medium} and sterilized. Seed was transferred to the slants (1 seed/tube). Tubes were capped and wrapped with parafilm, than labeled with the seed name, method of sterilization, and date of culturing. Tubes were stored at room temperature.

Six seedlings that were treated with 10% Clorox for 15 minutes are growing poorly on MS stage 1 gel slants; they germinated in two weeks, and have now been in culture over 9 weeks. Ten seedlings that were treated with 5% bleach for 70 minutes are growing very well on MS stage 1 gel slants; they also germinated in two weeks, and were in culture over nine weeks as of December 2009. These same sterile cultures, including sterile seedlings and vines, have been maintained for at least a year thereafter.

Alaska blueberry tissue from a second year (2010) was similarly sterilized and put into culture; these cultures are also viable after several weeks.

### Example 4

### American Cranberry (Vaccinium macrocarpon) Seed Culture

Vaccinium macrocarpon seed were prepped as follows for germination: Berries (obtained from Cranberry Hill Farm, Plymouth, Mass.) were surface sterilized by putting half in 5% Clorox for 60 minutes, and half in 50% Clorox for 20 minutes. Berries were rinsed 3 times in sterile deionized water.

Berries were cut in half and seeds were removed using sterile technique. Half of the berries were placed on Stage 1 liquid media {M527 Phytotechnology: Murashige Modified Multiplication Basal Medium} on filter paper in a sterile Petri dish. Tubes were filled with Stage 1 media.

Tubes were filled with Stage 2 media {M401 Phytotechnology: Murashige & Skoog Modified

Medium} with gellan gum {G434 Phytotechnology: Gellan Gum Powder} and sterilized. Half of the seed were put in these tubes; one seed was placed in each tube. Tubes were labeled with the seed name, method of sterilization, and date of culturing. Tubes were stored at room temperature.

After eight weeks in culture, there is no appreciable tissue growth. However, with several weeks additional incubation callus tissue culture has been established and maintained until at least December 2010.

### Example 5

### Frankincense (Boswellia sacra) Seed Germination

Boswellia sacra seed (obtained from MiniaTree, Tempe Ariz.) were prepped as follows for germination: Wings on seed were carefully removed with a scalpel. Seed was surface sterilized using three methods:

Sterilization Method #1: Seeds were put in 10% Clorox plus 2 drops of sterile 50% Tween 20 on a shaker for 10 minutes, then rinsed with sterile, deionized water under the hood until no bubbles formed.

Sterilization Method #2: Seeds were put in 3% hydrogen peroxide for 10 minutes on a shaker. The hydrogen peroxide was pipetted out, but the seeds were not rinsed.

Sterilization Method #3: Seeds were put in 10% Clorox plus 2 drops of sterile 50% Tween 20 on a shaker for 5 minutes, then rinsed with sterile, deionized water under the hood until no bubbles formed.

Tubes were filled with Stage 1 gel medium {M527 Phytotechnology: Murashige Modified Multiplication Basal Medium} and sterilized. Seed was transferred to the slants (1 seed/tube). Tubes were capped and wrapped with parafilm. Tubes were labeled with the seed name, date of initial culturing, and date of subculture. Tubes were stored in a 32.2° C. incubator in the dark.

One seed that was treated with hydrogen peroxide germinated, but then died after four weeks in culture. Clorox treatment is likely too harsh, as no Clorox treated seeds germinated. High seemed too harsh for the seedling; it withered after a few days in the heat.

Another set of frankincense seeds were germinated, using hydrogen peroxide sterilization only. After germination, seedlings were be moved to room temperature.

Frankincense plant sections from the germinated seed have been maintained in sterile culture.

### Example 6

### Giant Sequoia (Sequoiadendron giganteum) Seed Stratification and Germination

Sequoiadendron giganteum seed were prepped as follows for germination: Seed (Hirt's gardens, Medina Ohio) was surface sterilized using two methods:

Sterilization Method #1: 20 seeds were put in 10% Clorox plus 2 drops of sterile 50% Tween 20 on a shaker for 10 minutes, then rinsed with sterile, deionized water under the hood until no bubbles formed.

Sterilization Method #2: 20 seeds were put in 3% hydrogen peroxide for 10 minutes on a shaker. The hydrogen peroxide was pipetted out, but the seeds were not rinsed.

Seeds were cold stratified and germinated using two methods.

Stratification Method #1: 10 seeds surface sterilized with Clorox (Sterilization Method #1) were soaked overnight in sterile, deionized water in a sterile Petri dish. 10 seeds surface sterilized in hydrogen peroxide (Sterilization Method #2) were soaked overnight in sterile, deionized water in a sterile Petri dish. Two Petri dishes were filled sand and autoclaved. Sterile filter paper was placed on the sand and was moistened with sterile water. 10 seeds surface sterilized with Clorox (Sterilization Method #1) were put in one Petri dish on top of the filter paper and stored at 4° C. for 60 days. 10 seeds surface sterilized with hydrogen peroxide (Sterilization Method #2) were put in the other Petri dish on top of the filter paper and stored at 4° C. for 60 days. Seeds were removed from the cold and incubated in these Petri dishes at room temperature with 12 hours of fluorescent light and 12 hrs of darkness. When filter paper began to dry, more sterile deionized water was added under the hood as needed to keep seed moist.

Stratification Method #2: 10 seeds surface sterilized with Clorox (Sterilization Method #1) were placed in a sterile 250 ml flask containing sterile deionized water, covered with sterile foil and kept at 4 degrees C. 10 seeds surface sterilized in hydrogen peroxide (Sterilization Method #2) were placed in a sterile 250 ml flask containing sterile deionized water, covered with sterile foil and kept at 4° C. The seeds were allowed to float on the water for 40 days without overlapping.

Tubes were filled with Stage 1 gel medium {M527 Phytotechnology: Murashige Modified Multiplication Basal Medium} and sterilized. Germinated seed was transferred to the slants (1 seed/tube). Tubes were capped and wrapped with parafilm, then labeled with the seed name, method of sterilization, and date of culturing. Tubes were stored at room temperature.

11 seedlings are growing well on MS stage 2 gel slants that received the cold stratification method 2 with Clorox treatment. These seeds germinated one week after they were put in culture following cold stratification treatment and have now been in culture over 4 weeks. Four seedlings are growing (not as healthy as the first set) on MS stage 1 gel slants that received cold stratification method 2 with hydrogen peroxide treatment. These seeds germinated 1 week after they were put in culture following cold stratification treatment; they have been in culture more than several months.

### Example 7

### Glossy Black Huckleberry (Vaccinium mebranaceum) Seed Culture Plan

Vaccinium mebranaceum seed were prepped as follows for germination: Seed (obtained from Alaska Blues, LLC Calder Mt. Tongass Nat'l Rain Forest, Alaska) was surface sterilized in 3% hydrogen peroxide for 5 minutes. The hydrogen peroxide was removed by a vacuum pump and a filter sterilization unit. Seed was rinsed with sterile deionized water and vacuum pumped to dry.

Tubes were filled with Stage 1 gel medium {M527 Phytotechnology: Murashige Modified Multiplication Basal Medium} and sterilized. Seed was transferred to the slants (1 seed/tube). Tubes were capped and wrapped with parafilm, then labeled with the seed name, method of sterilization, and date of culturing. Tubes were stored at room temperature.

Seven seedlings that were treated with 5% Clorox for 70 minutes are growing well on MS stage 1 gel slants; they germinated in two weeks, and have now been in culture over 9 weeks. Two seedlings that were treated with 10% Clorox for 15 minutes are growing well on MS stage 1 gel slants; they germinated in two weeks, and have now been in culture over 9 weeks.

Using this procedure, callus and multiple sterile vines/seedling have been produced and maintained in culture.

The same procedure has been repeated with a second batch of Alaska Huckleberries, also with success.

### Example 8

### Coffee Bean (Coffea arabica) Germination Plan

Coffea arabica whole cherries (of all stages Green, Semi-Ripe and Ripe) (obtained from Lyman Kona Coffee Farms, Koilha-Kona Hi.) are prepped as follows for germination: Coffee cherries are surface sterilized using 10% Clorox plus 2 drops of sterile 50% Tween 20 on a shaker for 10 minutes, then rinsed with sterile, deionized water under the hood until no bubbles form.

Sterile tubes are filled with liquid Stage 1 media {M527 PhytoTechnology: Murashige Modified Multiplication Basal Medium} and sterilized. Sterile filter paper “rafts” (bent into an “M” shape to cradle the bean better) are placed in the medium with the middle portion of the “M” just touching the media. The coffee cherries are opened with a sterile scalpel and the whole peel removed. The whole bean (for most coffees) contains two seeds, so the bean is split in half along the center membrane. Each half of the bean is placed into one sterile tube and sealed with parafilm

Another two batches (containing each stage G, SR and R) are sterilized and the whole bean bisected as described in the above method. These beans are placed in either Stage I media {M527 PhytoTechnology: Murashige Modified Multiplication Basal Medium} or Stage II media {M401 PhytoTechnology: Murashige and Skoog modified medium} sterile slants that have been solidified with 2.25-25% GeRite {Gellem Gum}.

The beans are buried with about 95% of the surface of the bean submerged in the media. Seeds are subcultured to new tubes with appropriate media and new sterile filter paper “rafts” every 10 days. Tubes are labeled with the seed name, date of initial culturing, and date of subculture (when performed).

Using this procedure, sterile cultures including sterile callus have been established.

### Example 9

### Alaska Paper Bark Birch (Betula neoalaskana) Seed Germination Plan

Betula neoalaskana seed are prepped as follows for germination: Seed are surface sterilized, for instance using these two methods:

Sterilization Method #1: 5 seeds are put in 10% Clorox plus 2 drops of sterile 50% Tween 20 on a shaker for 10 minutes, then rinsed with sterile, deionized water under the hood until no bubbles form.

Sterilization Method #2: Three days after the first method is performed, 5 seeds are put in 3% hydrogen peroxide for 10 minutes on a shaker. The hydrogen peroxide is pipetted out, but the seeds are not rinsed. Five seeds surface sterilized with Clorox (Sterilization Method #1) are soaked overnight in sterile, deionized water in a sterile Petri dish. Five seeds surface sterilized in hydrogen peroxide (Sterilization Method #2) are soaked overnight in sterile, deionized water in a sterile Petri dish.

Tubes are filled with Stage 1 gel medium {M527 Phytotechnology: Murashige Modified Multiplication Basal Medium} and sterilized. Seed is transferred to the slants (1 seed/tube). Tubes are capped and wrapped with parafilm, and labeled with the seed name, method of sterilization, and date of culturing. Tubes can then be stored at room temperature.

### Example 10

### Cedar-of-Lebanon (Cedrus libani) Seed Germination Plan

Cedrus libani seed (obtained from Whatcom Seed, Eugene Oreg.) are prepped as follows for germination: Seed is surface sterilized using, for instance, these two methods:

Sterilization Method #1: 20 seeds are put in 10% Clorox plus 2 drops of sterile 50% Tween 20 on a shaker for 10 minutes, then rinsed with sterile, deionized water under the hood until no bubbles form.

Sterilization Method #2: Three days after the first method is performed, 20 seeds are put in 3% hydrogen peroxide for 10 minutes on a shaker. The hydrogen peroxide is pipetted out, but the seeds are not rinsed.

Seeds are cold stratified and germinated. 20 seeds surface sterilized with Clorox (Sterilization Method #1) are soaked overnight in sterile, deionized water in a sterile Petri dish. Likewise, 20 seeds surface sterilized in hydrogen peroxide (Sterilization Method #2) are soaked overnight in sterile, deionized water in a sterile Petri dish. Two Petri dishes are filled sand and autoclaved. Sterile filter paper is placed on the sand and moistened with sterile water. 20 seeds surface sterilized with Clorox (Sterilization Method #1) are put in one Petri dish on top of the filter paper and stored at 4° C. for 21 days. 20 seeds surface sterilized with hydrogen peroxide (Sterilization Method #2) are put in the other Petri dish on top of the filter paper and stored at 4° C. for 21 days.

Seeds are removed from the cold and incubated in these Petri dishes at room temperature with 12 hours of fluorescent light and 12 hours of darkness. When filter paper begins to dry, more sterile deionized water is added under the hood as needed to keep seed moist.

Tubes are filled with Stage 1 gel medium {M527 Phytotechnology: Murashige Modified Multiplication Basal Medium} and sterilized. Germinated seed is transferred to the slants (1 seed/tube). Tubes are capped and wrapped with parafilm, then labeled with the seed name, method of sterilization, and date of culturing.

### Example 10

### Coastal Redwood (Sequoia semervirens) Seed Germination Plan

Sequoia sempervirens seed (obtained from Whatcom Seed, Eugene Oreg.) are prepped as follows for germination. Seed is surface sterilized using two methods:

Sterilization Method #1: 20 seeds are put in 10% Clorox plus 2 drops of sterile 50% Tween 20 on a shaker for 10 minutes, then rinsed with sterile, deionized water under the hood until no bubbles form.

Sterilization Method #2: 20 seeds are put in 3% hydrogen peroxide for 10 minutes on a shaker. The hydrogen peroxide is pipetted out, but the seeds are not rinsed.

Seeds are germinated using two methods.

Germination Method #1: 10 seeds surface sterilized with Clorox (Sterilization Method #1) are soaked overnight in sterile, deionized water in a sterile Petri dish. 10 seeds surface sterilized in hydrogen peroxide (Sterilization Method #2) are soaked overnight in sterile, deionized water in a sterile Petri dish. Two Petri dishes are filled with sand and deionized water, and autoclaved. Sterile filter paper was placed on the wet sand. 10 seeds surface sterilized with Clorox (Sterilization Method #1) are put in one Petri dish on top of the filter paper and stored at room temperature until germination. 10 seeds surface sterilized with hydrogen peroxide (Sterilization Method #2) are put in the other Petri dish on top of the filter paper and stored at room temperature until germination. When filter paper began to dry, more sterile deionized water is added under the hood as needed to keep seed moist.

Tubes are filled with Stage 1 gel medium {M527 Phytotechnology: Murashige Modified Multiplication Basal Medium} and sterilized. Germinated seed is transferred to the slants (1 seed/tube). Tubes are capped and wrapped with parafilm, then labeled with the seed name, method of sterilization, and date of culturing. Tubes are stored at room temperature.

Germination Method #2: Two Petri dishes are filled with sand and deionized water, and autoclaved. 10 seeds surface sterilized with Clorox (Sterilization Method #1) are placed directly on the wet, sterile sand. 10 seeds surface sterilized in hydrogen peroxide (Sterilization Method #2) are placed directly on the wet, sterile sand. When filter paper began to dry, more sterile deionized water is added under the hood as needed to keep seed moist. Tubes are filled with Stage 1 gel medium {M527 Phytotechnology: Murashige Modified Multiplication Basal Medium} and sterilized. Germinated seed is transferred to the slants (1 seed/tube). Tubes are capped and wrapped with parafilm, the tubes are labeled with the seed name, method of sterilization, and date of culturing. Tubes are stored at room temperature.

### Example 11

### Great Basin Bristlecone pine (Pinus longaeva) Seed Germination Plan

Pinus longaeva seed is prepped as follows for germination:

Sterilization Method #1: 10 seeds are put in 10% Clorox plus 2 drops of sterile 50% Tween 20 on a shaker for 10 minutes, then rinsed with sterile, deionized water under the hood until no bubbles formed.

Sterilization Method #2: Three days after the first method was performed, 10 seeds are put in 3% hydrogen peroxide for 10 minutes on a shaker. The hydrogen peroxide is pipetted out, but the seeds are not rinsed.

10 seeds surface sterilized with Clorox (Sterilization Method #1) are soaked overnight in sterile, deionized water in a sterile Petri dish. 10 seeds surface sterilized in hydrogen peroxide (Sterilization Method #2) are soaked overnight in sterile, deionized water in a sterile Petri dish. Tubes are filled with Stage 1 gel medium {M527 Phytotechnology: Murashige Modified Multiplication Basal Medium} and sterilized. Seed is transferred to the slants (1 seed/tube). Tubes are capped and wrapped with parafilm, then labeled with the seed name, method of sterilization, and date of culturing. Tubes are stored at room temperature in the dark.

### Example 12

### Mediterranean Olive (Olea europaea) Seed Germination Plan

Olea europaea seed (obtained from Whatcom Seed, Eugene, Oreg.) are prepped as follows for germination: Seed is surface sterilized using two methods.

Sterilization Method #1: 5 seeds are put in 10% Clorox plus 2 drops of sterile 50% Tween 20 on a shaker for 10 minutes, then rinsed with sterile, deionized water under the hood until no bubbles formed.

Sterilization Method #2: Three days after the first method is performed, 5 seeds are put in 3% hydrogen peroxide for 10 minutes on a shaker. The hydrogen peroxide is pipetted out, but the seeds are not rinsed.

Seed coats are scarified using a sterile scalpel to cut the seed coat in several places. Seeds are cold stratified and germinated. 5 seeds surface sterilized with Clorox (Sterilization Method #1) are soaked overnight in sterile, deionized water in a sterile Petri dish. 5 seeds surface sterilized in hydrogen peroxide (Sterilization Method #2) are soaked overnight in sterile, deionized water in a sterile Petri dish. Two Petri dishes are filled sand and autoclaved. Sterile filter paper is placed on the sand and was moistened with sterile water. 5 seeds surface sterilized with Clorox (Sterilization Method #1) are put in one Petri dish on top of the filter paper and stored at 4° C. for 90 days. 5 seeds surface sterilized with hydrogen peroxide (Sterilization Method #2) are put in the other Petri dish on top of the filter paper and stored at 4° C. for 90 days.

Seeds are removed from the cold and incubated in these Petri dishes at room temperature with 12 hours of fluorescent light and 12 hrs of darkness. When filter paper begin to dry, more sterile deionized water is added under the hood as needed to keep seed moist.

Tubes are filled with Stage 1 gel medium {M527 Phytotechnology: Murashige Modified Multiplication Basal Medium} and sterilized. Germinated seed is transferred to the slants (1 seed/tube).

Tubes are capped and wrapped with parafilm, labeled with the seed name, method of sterilization, and date of culturing.

### Example 13

### Sea Buckthorn (Hippophae rhamnoides) Seed Germination Plan

Hippophae rhamnoides seed are prepped as follows for germination. Seed is surface sterilized using two methods:

Sterilization Method #1: 5 seeds are put in 10% Clorox plus 2 drops of sterile 50% Tween 20 on a shaker for 10 minutes, then rinsed with sterile, deionized water under the hood until no bubbles formed.

Sterilization Method #2: Three days after the first method was performed, 5 seeds are put in 3% hydrogen peroxide for 10 minutes on a shaker. The hydrogen peroxide is pipetted out, but the seeds are not rinsed.

Seeds are cold stratified and germinated. 5 seeds surface sterilized with Clorox (Sterilization Method #1) are soaked overnight in sterile, deionized water in a sterile Petri dish. 5 seeds surface sterilized in hydrogen peroxide (Sterilization Method #2) are soaked overnight in sterile, deionized water in a sterile Petri dish. Two Petri dishes are filled sand and autoclaved. Sterile filter paper is placed on the sand and moistened with sterile water. 5 seeds surface sterilized with Clorox (Sterilization Method #1) are put in one Petri dish on top of the filter paper and stored at 4° C. for 90 days. 5 seeds surface sterilized with hydrogen peroxide (Sterilization Method #2) are put in the other Petri dish on top of the filter paper and stored at 4° C. for 90 days.

Seeds are removed from the cold and incubated in these Petri dishes at room temperature with 12 hours of fluorescent light and 12 hrs of darkness. When filter paper begins to dry, more sterile deionized water is added under the hood as needed to keep seed moist.

Tubes are filled with Stage 1 gel medium {M527 Phytotechnology: Murashige Modified Multiplication Basal Medium} and sterilized. Germinated seed is transferred to the slants (1 seed/tube). Tubes are capped and wrapped with parafilm, then labeled with the seed name, method of sterilization, and date of culturing.

### Example 14

### Culture of Strawberry {Fragaria virginiana} and Blueberries {Vaccinium formosum}

The firmest and youngest berries (obtained from a private residence in Virginia Beach, Va.) were selected and placed in a sterile tube with a sterile solution of 10% bleach and 1 drop of Tween 50. The tubes were gently agitated by hand (can also be placed on a shaker platform) for ten minutes. Sterilization times and concentrations may be increased in an inverse relationship if contamination of initial cultures is observed, until sterile cultures are generated. For example if the bleach concentration is increased to 20% then the agitation time should be decreased to 5 minutes. Berries were removed from sterilization solution and rinsed with autoclaved H₂O until all suds were gone (minimum 3× rinse) and placed in a sterile Petri dish.

Using a sterile scalpel the berries were bisected along all axes (vertical, horizontal, etc.), internal “core” sections and thin sections were taken. This gives the most cell types the potential to generate callus tissue. Using sterile forceps the sections were placed into the media (slant tube or liquid flask) ensuring the cut surfaces were fully submerged into the media to allow nutrient absorption.

These materials were put into culture; none survived past two weeks due to death in culture or contamination. These are not the same blueberries as the Alaskan culture mentioned above.

### Example 15

### Culture of Camellia sinensis, Coffea arabica and Vaccinium formosum meristems

Several active growth (green and non woody) meristems (both horizontal and apical) were selected from each of Camellia sinensis, Coffea arabica (obtained from Lyman Farms, Kona, Hi.) and Vaccinium formosum live plants (obtained from private residence in Virginia Beach, Va.). A larger section then needed was cut so it could be trimmed down after sterilization (this preserves tissue from the bleaching agents needed to sterilize). The cut sections were placed in sterile tubes containing a sterile 10% bleach solution and 1 drop of sterile Tween 50. They were agitated by hand for ten minutes or 30 minutes.

The cuttings were removed from the tubes and rinsed in a separate sterile tube with autoclaved H₂O until the suds were gone (minimum 3× rinse). After rinsing the material was placed in a sterile Petri dish and any leaves were removed from the meristems at the leaf base (leaving only meristem).

Using sterile forceps the cut material was placed into the solid media (completely submerging the cut section). Tubes were sealed with parafilm and placed in racks at room temperature.

The lateral meristems are used for microdivision (propagation of sterile cultures by microcuttings of sterile cultures to generate new cultures). This is only done with lateral meristems; apical meristems are reserved for generation of callus tissue.

Green Tea (C. sinensis) flower buds attempted in this manner were all contaminated and the cultures were lost.

### Example 16

### Culture of Orchid (Phalaenopsis 09.745) Stem

A sterile cultured stem from a phalaenopsis orchid (designated 09.745) was selected. Sections were cut at each “node” (area where healthy leaves connect to the stem) using a sterile blade. The cut sections were placed in sterile tubes containing autoclaved water and rinsed of all excess culture media and placed into Stage II (previously described) media for generation of callus tissue. The cut surface of each section or node was placed below the surface of the solid media, and a diced section of node was placed into liquid media and unto a room temperature orbital shaker platform.

Node development has been observed with this culture.

### Example 17

### Culture of Additional Phalaenopsis Samples

Additional orchid samples, listed below, were used to obtain sterile cultures. Stem nodes were prepared in accordance with the procedure in Example 16. In order to sterilize other plant parts (sepals, petals, leaves and pollen), they were treated with 100% ETOH for 5 seconds, then a thin section was cut from each part of the plant listed and placed in MS Stage I media.

| Species (and Variety) | Name | Ploidy |
|---|---|---|
| Phalaenopsis amabilis | Fancy Pearl | |
| Phalaenopsis amabilis var. compactum | Mini Pearl (Tying amabilis) | |
| Phalaenopsis amabilis var. fromosanum | Fantastic 4N | 4N |
| Phalaenopsis amabilis (NF1370) | 4N Strain | 4N |
| Phalaenopsis Sogo Yukidian | Japan (Yukimai x Taisuco Koehdian) | |
| Phalaenopsis amabilis var. formosanum | Amabilis ‘snow summit’ HCC/AOS | |
| | x Amabilis ‘Angel’ HCC/AOS | |
| Phalaenopsis Aphrodite var. formosanum | ‘Ben Yu’ AM/AOS | |

Node development has been observed with these cultures.

### Example 18

### Culture of Sterile Neofinetia Tissue

The following two orchid species were put in culture: (1) Neo falcata (white) ‘Furan’ X Self (protocorm-like material with small leaves) aka 06-1499; and (2) Neo falcata (white) ‘Giant Classic Snowflake” X Neo falcata (white) ‘Giant Classic Egret’ (late stage protocorms) aka 08-3797.

Each of these orchid species was already in sterile culture in orchid medium. Samples from each were removed and chopped with a sterile scalpel to wound and induce callus formation. About 2-3 tsp (spoonula scoops) of each species type was used for this process. Chopped pieces were placed in 250 ml Erlenmeyer flasks containing approximately 50 ml liquid Stage 2 medium. Flasks were placed in a shaker incubator at a temperature of 26 C and rotation (flat, level) of 55 rpm. After 3 weeks and 7 weeks of culture, the medium on all flasks was replaced with fresh liquid Stage 2 medium.

After 9.5 weeks, no development of #1 (06-1499) had been observed.

After 9.5 weeks, #2 (08-3797) is still in culture. At seven weeks, callus-like tissue was apparent at the ends of many cut areas. Some pieces were pulled from the liquid culture and placed on stage 2 medium slants (after 7 weeks of culture) so the development could be observed more closely. This is ongoing.

These pieces may be used for the BA:NAA concentration experiment described in Example 21.

Using this procedure, at least three callus cultures have been formed and sustained.

### Example 19

### Culture of Purple Muscadine Grapes

The firmest and youngest berries (grapes) (obtained from Paulk Vineyards, Wray, Ga.) were selected and placed in a sterile tube with a sterile solution of 10% bleach and 1 drop of Tween 50. The tubes (containing one Ripe to Very Ripe grape each) were gently agitated by hand for ten minutes and two tubes (each containing one grape) were agitated for 20 minutes.

Grapes were removed from sterilization solution and rinsed with autoclaved H₂O until all suds were gone (minimum 3× rinse) and placed in a sterile Petri dish. Using a sterile scalpel and technique the grapes were bisected along all axes (vertical, horizontal, etc.), internal “core” sections and thin sections were taken. This gives the most cell types the potential to generate callus tissue. The grape seeds were also removed and cultured in sterile solid media, and in liquid media on folded filter paper “rafts” inside sterile tubes to generate a sterile stock vine of grapes for future testing applications.

Using sterile forceps, the cut sections of the grape fruit were placed into the media (slant tube or liquid flask), ensuring the cut surfaces were fully submerged into the media to allow nutrient absorption. Diced sections of the grape fruit (skin and pulp), as well as diced sections of grape seeds, were also prepared and placed into liquid media on an orbital shaker at room temperature.

Seed, pulp, peel, and ¼ grape sections have produced no visible callus or seedlings after more than 13.5 weeks in culture.

### Example 20

### Culture of Additional Plant Tissues

Additional plant seeds and/or tissue were put into culture using methods essentially similar to those described above. Characteristics of the starting plant material, sterilization techniques, and results are provided in the following table:

| Plant | Genus/species | Variety | Source | Results |
|---|---|---|---|---|
| Mini Red Onion | Allium cepa | Mini Red Marble | Johnny's Selected Seeds, | Two sterile |
| seeds* | | F1 Hybrid | Winslow; Maine | cultures obtained |
| Amethyst Basil | Ocimum | Amethyst | Johnny's Selected Seeds, | Contaminated |
| seeds* | basilicum | Improved | Winslow; Maine | |
| Amethyst Basil | Ocimum | Amethyst | Johnny's Selected Seeds, | Contaminated |
| leaf sections | basilicum | Improved | Winslow; Maine | |
| Redbor Kale | Brassica | Redbor F1 | Johnny's Selected Seeds, | Two plant |
| seeds* | oleracea | Hybrid | Winslow; Maine | cultures obtained, |
| | Acephala | | | not in great health |
| | Group | | | |
| Alpine | Fragaria vesca | semper florens | Edible Landscaping, | Two sterile |
| Strawberry parts | | (Red Wonder | Afton VA | cultures obtained |
| | | Alpine) | | |
| Purple Potato | Solanum | Adirondak Blue | Johnny's Selected Seeds, | Three sterile |
| sections and | tuberosum | | Winslow; Maine | cultures obtained |
| chunks | | | | |
| Sunflower | Helianthus | Big Smile | Johnny's Selected Seeds, | No cultures |
| petals, leaves | annuus | (dwarf) | Winslow; Maine | obtained |
| and parts | | | | |
| Pungo grown | Rubus | | Stoney's Produce 1st | Multiple sterile |
| Blackberry | | | Colonial Rd, Virginia | cultures obtained |
| sections | | | Beach, VA: Fruit from | |
| | | | Pungo, VA | |
| Black Pearl | Solanum | Black Pearl | Burpee & Co, | Contaminated |
| Tomato sections | lycopersicum | | Warminster; PA | |

*Seeds were sterilized using 3% H₂O₂ for 10 minutes before they were placed in standard stage I media.

### Example 21

### System for Testing Cytokinin/Auxin (e.g., BA:NAA) Concentration Combinations for Optimizing Support of Cultured Callus Tissue

This example describes one system for optimizing tissue culture conditions for the production of callus or plant part regeneration by varying the amount and proportion of an auxin (e.g., NAA) and a cytokinin (e.g., BA). This example is described with regard to orchid tissue, but the same or similar experiments can be carried out with any plant tissue culture, including the specific cultures described herein. Likewise, other auxins and cytokinins can be substituted for the ones described here.

By way of example, cultures of two orchids (Neo falcata (green) V. Hisui ‘Jade’ X Neo falcata (green) V. Hisui ‘Jade’ (protocorms)) and (Neo falcata (white) ‘Giant Classic Snowflake” X Neo falcata (white) ‘Giant Classic Egret’ (protocorm-like plant material)) are used. Alternatively, the cultures produced in Example 17 may be used.

BA and NAA will be added to the MS Basal Medium (Murashige and Skoog Basal Medium w/vitamins (Phytotechnology M519)) according to the concentrations demonstrated on the matrix (below) and processed as gelled slants. Plant material will be placed on the slant surface of each of the concentrations in the matrix below and observed for growth to determine which BA/NAA concentration combination best supports callus growth.

Matrix for Study of BA/NAA Concentrations with Callus Cultures

| | mg/L | mg/L | mg/L | mg/L | mg/L |
|---|---|---|---|---|---|
| NAA | 0 | 0 | 0 | 0 | 0 |
| BA | 0 | 0.5 | 1.0 | 1.5 | 2.0 |
| NAA | 0.5 | 0.5 | 0.5 | 0.5 | 0.5 |
| BA | 0 | 0.5 | 1.0 | 1.5 | 2.0 |
| NAA | 1.0 | 1.0 | 1.0 | 1.0 | 1.0 |
| BA | 0 | 0.5 | 1.0 | 1.5 | 2.0 |
| NAA | 1.5 | 1.5 | 1.5 | 1.5 | 1.5 |
| BA | 0 | 0.5 | 1.0 | 1.5 | 2.0 |

BA = 6-benzylaminopurine (Phytotechnology N600) (a cytokinin)
NAA = a-napthaleneneacetic acid (Phytotechnology B800) (an auxin)

### Example 22

### System for Culture of Coffee Cherries and Beans

This example provides a representative system for the in vitro culture of coffee cherries and beans.

Careful records were kept of the source, date, condition, etc. of the original plant material. For instance, the shipment was recorded (in a Material Logbook) the day the shipment is received, including a description of the material, the location it was received from in as much detail as possible, the amount of material received (if in various stages, indicate number/amount of material per stage), and a designation the receiving/preparing individual. This allows results to be correlated with plant condition, tissue, and source.

Material was sorted by stage (e.g., Unripe {U}, Semi Ripe {SR}, Ripened {R}, and Very Ripe {VR}) and 5-10 of the best looking cherries were selected for a photographic record. Representative photos included: Material Grouped by Stage; Material showing each stage in one photo; Bean Material (unroasted, roasted and dark roasted) grouped together; Fresh bisected material at each stage.

A selected portion of each stage of the material (optionally, more than 25% but less than 75%) was flash frozen with liquid nitrogen (wrapped in foil when needed) by immersion. All frozen material was collected by stage, labeled with material name, date received, location received from, stage of material and date frozen and placed in the −80° C. freezer. Stored thus, it can be kept indefinitely.

Primary tissue cultures were started from: 1) whole coffee cherry from all stages (U, SR, R, and VR}, 2) the bean (removed freshly from the cherry) for the SR stage, and 3) the Pulp+Peel (no bean) from the SR stage. Other cultures were attempted (until material from each stage was depleted); these included: Pulp+Peel from any other stages, beans from any other stages. The following is a representative matrix that enables easy tracking of different culture types based on source material and stage (though other organizational systems can be employed):

Coffee Bean Culture

| | Whole | Bean | Pulp | Pulp | Peel | Root | Apical | |
|---|---|---|---|---|---|---|---|---|
| | cherry | only | & Peel | only | only | Tip | Meristem | Flower |
| Green | | | | | | | | |
| Semi-ripe | | | | | | | | |
| Ripe | | | | | | | | |
| Ripe Ripe | | | | | | | | |
| Light Roasted | | | | | | | | |
| Dark Roasted | | | | | | | | |

Material for primary cultures was sterilized by placing the material in 10% bleach and sterile H₂O with 1-2 drops of Tween 20 for 10 minutes on a shaker (or gently shaken by hand). The material was rinsed, under the hood, with sterile water until all the Tween is removed (no more bubbles/foam; minimum of 3× rinse). The plant material was removed using sterile forceps and placed in sterile Petri dish for manipulation.

Four tubes (per individual piece of material) of Stage I media {M527 Phytotechnology: Murashige Modified Multiplication Basal Medium} were labeled with the material, date of initial culturing, stage and date of subculture if applicable. Using sterile scalpel and aseptic technique, the coffee material was sliced in half, the bean removed and placed into one of the culture tubes (this tube contains the liquid media and a filter paper raft as described herein). One of the remaining halves was placed cut side down and buried deep into the Stage I media slant. The other half was bisected (leaving two quarters of a whole berry) and one of the quarters was placed into a Stage I media slant (making sure all cut edges are buried in the media). The final fourth was sectioned into several thin sections and placed into a Stage I media slant, covering as much of the section as possible while still allowing for air exchange.

A second piece of material was bisected and the bean removed. The bean was then cut up into quarters and placed into a 250 mL flask containing liquid Stage I media. The remaining pulp/peel was cut into quarters as well and placed in a 250 ml flask containing liquid Stage I media. Both flasks were placed at the lowest rotation speed inside the orbital shaker/incubator.

These preparation steps were repeated for all development stages until a sufficient number of cultures were generated or viable material was depleted.

All flasks were sealed with sterile foil and Parafilm, and all culture tubes with Parafilm. Cultures were observed for growth daily and subcultured every 10 days. Once cell culture was established as sterile and callus tissue is generated, it was switched to Stage II media {M401 Phytotechnology: Murashige and Skoog Modified Medium}

Once the culturing was completed, material was sterilized for drying. When sterile, it was placed on filter paper in a flask and into an incubator/oven or into food dehydrator (when available). Other samples were cut into fine pieces and left to dry.

Using this protocol, germination-type activity was noted after 4 weeks in culture of an equatorial cross section of a whole coffee cherry; first two weeks in stage 1, second 2 weeks in stage 2. This non-whole, chunk of bean tissue produced a continuing-in-culture plant (now at 11.5 weeks post initiation of culture), a plantlet with roots, shoot, and leaves in culture.

### Example 23

### Microarray Analysis of Gene Expression in Human Skin Fibroblasts Exposed to Extract from Cultured Coffee Cherry Cells

This example describes an in vitro analysis of the effects of an extract from cultured coffee cherry on human skin fibroblasts using a focused microarray for selected genes related to lifespan and health.

Green coffee cherry (obtained from Lyman Coffee Farms, Kona, Hi.) was treated with 15% bleach for 10 minutes, rinsed in sterile water, treated 5 minutes in 3% hydrogen peroxide, and rinsed again with sterile water. The sterilized coffee cherry was then aseptically opened and the bean removed and quartered. Quarters were put on stage 1 slants. Two weeks later, they were moved to stage 2 slants. This “cultured” bean was removed from culture and used for extraction at week 9 from initial culture date.

Approximately 60 mg of coffee cherry bean tissue that had been in semi-solid phase culture for approximately 9 weeks was homogenized in 500 μl cold 100% ethanol and allowed to extract overnight at 4° C. The next day, the homogenate was vortexed and allowed to extract at room temperature for several hours. The mixture was allowed to settle for 5 minutes room temperature so heave cellular solids sedimented, and the supernatant was drawn off as bean extract.

Human skin fibroblasts (AG07999, Coriell Institute) were seeded at near confluence in 6-well dishes 24 hours before exposure to the bean extract. The culture medium was MEM, 10% FBS, 2 mM glutamine, 1× Glutamax I at a volume of 5 ml per well. After 24 hours the wells were aspirated and received 3 ml of test or control condition (in duplicate). The experimental phase medium was the same as the culture medium, except the FBS was reduced to 1%. The test conditions were: (1) 15 μl undiluted bean extract/well; and (2) 3 μl undiluted bean extract/well+12 μl 100% ethanol The control wells received 15 l 100% ethanol. The final ethanol concentration in each well was 0.5%.

For comparison, parallel sample of fibroblasts were treated with the coffee cherry extract COFFLEBERRY® (Lot#02480000X5729; VDF FutureCeuticals, Inc., Momence, Ill.; derived from Mexican coffee plants) or chlorogenic acid, as described previously (e.g., U.S. application Ser. No. 12/629,040 or PCT/US2009/066294 (both filed Dec. 1, 2008 and incorporated herein in its entirety), published as US-2010-0173024 on Jul. 8, 2010.

This experiment was performed twice. The extract was stored frozen at −20° C. between experiments. The fibroblast monolayers were visually inspected under an inverted microscope before RNA isolation at 24 hours, and appeared healthy.

RNA was isolated using the manufacturer's protocol for the RT² qPCR grade RNA Isolation Kit (SABiosciences, Fredrick Md.). The RNA was then examined for purity and quantity using a spectrophotometer and the 260/280 nm ratio. An equal amount of RNA was synthesized into cDNA using the RT² First Strand Synthesis Kit (SABiosciences, Fredrick Md.) in accord with the manufacturer's protocol. The resulting samples were then loaded into a custom human microarray and analyzed on a BioRad iCycler.

### Results

Significant gene expression changes were observed. Both concentrations of bean extract demonstrated gene expression changes in the 4-5 range. Genes showing an appreciable change in gene expression include interleukins and HSPA.

COL3A1 is upregulated in the higher concentration of bean extract, and even more upregulated in the 5:1 dilution. This dosage response is inverse and paradoxical. COL3A1 is critical in wound healing—and thus the discovery illustrated here that an extract of coffee cherry tissue can tremendously increase expression of COL3A1 provides clear evidence that such extracts (and possibly specific components purified from such extracts) can be used to promote wound healing. In addition, the POT1 gene is also upregulated at both concentrations of bean extract. POT1 is a recognized telomere protection gene, thus illustrating that the described coffee tissue extract is effective at stimulating telomere protection.

There appears to be a very different ‘fingerprint’ of this extract than of the coffeecherry (powder obtained from VDF) or chlorogenic acid. There also seems to be a dose response effect, with the 1:5 dilution in some cases having significantly greater gene expression changes,

As illustrated in the data below, COL3 and COL1 to a lesser degree are upregulated, but there are also changes with FOS and JUN (API—recognized as an important site of action of RetinA), and NFKB which impact collagen synthesis. The cultured coffee bean extract also increases expression of TGFB1 and epidermal growth factors, which are all classic genes for anti-aging in skin, making it look younger.

VEGF is consistently up regulated. This ties into improved wound healing and anti aging. Some key interleukins are down regulated; this can be beneficial because an anti inflammatory agent may also be anti-aging.

Interestingly, the expression of all four of the SIRT genes is impacted.

DNA repair and telomere maintenance genes are upregulated, including PARP1, PARP3, TERF2, TINF1, and NEIL1. Likewise, other genes linked to repair, maintenance, anti oxidative stress, etc. are up—such as SHC1, NADSYN1,HSPA1A, HSPA1B, TP53, SOD2, CASP2, MAPK14, IL8, SIRT2, BCL2L1, and HMOX1. TOMM40 and some other mitochondrial-related genes display altered expression also.

APOE and some other genes related to atherosclerosis are also impacted (recalling of course that these are human skin fibroblast cells and not blood vessel endothelial cells).

This extract described in this example has some apparently very potent effects on gene expression tied closely to lifespan/longevity, healthy/anti aging and ‘protect/defend/repair’. This profile is different than was seen with standard coffee cherry extract. This difference clearly validates the approach described herein, of using tissue culture to obtain different metabolite profiles from what might otherwise seem to be the “same” source.

In the following table, statistically significant (p≦0.05) differences are marked with an asterisk (*).

Fold Change in Gene Expression from Cultured Human Fibroblasts 24 Hours After Exposure to the Indicated Compositions

| | Cultured | | | |
|---|---|---|---|---|
| | Coffee | Cultured Coffee | 0.0001% | 0.00005% |
| Gene | Bean | Bean Extract | Coffee | Chlorogenic |
| Symbol | Extract | (1:5 dilution) | Cherry | Acid |
| PARP1 | 2.8 | −2.4 | −1.3 | 2.1 |
| IGF1 | −1.7 | −2.0 | 3.1 | −1.0 |
| SHC1 | 3.5 | 3.5 | −1.2 | 3.3 |
| IL1A | −1.5 | −1.9 | −2.7 | −1.1 |
| NADSYN1 | 3.0 | 2.6 | −1.4 | 2.5 |
| PARP2 | 1.4 | 1.7 | −1.6 | 1.9 |
| IGF2 | −1.7 | −2.0 | 3.1 | −1.0 |
| IFI44 | 1.3 | 1.5 | −1.9 | 1.6 |
| IL6 | 1.2 | 1.5 | −2.1 | 1.0 |
| BAX | 2.3 | 2.5 | −1.1 | 3.8 |
| PPARG | −1.7 | −2.0 | 3.1 | −1.0 |
| CLK1 | 1.1 | 1.1 | −1.6 | 1.8 |
| CREBBP | 1.3 | 1.6 | −1.8 | 2.1 |
| IL10 | −1.7 | −2.0 | 3.1 | −1.0 |
| COX1 | 1.2* | 1.4* | −1.9 | 1.8 |
| APOE | 2.5 | 2.5 | −2.2 | −1.0 |
| TERT | −1.7 | −2.0 | 3.1 | −1.0 |
| CYP19A1 | 2.5 | −1.2 | 3.1 | 2.5 |
| TNF | −1.7 | −2.0 | 3.1 | −1.0 |
| PTGS2 | −1.3 | −1.1 | −3.4 | 2.2 |
| HLA-DRA | −1.7 | −2.0 | 3.1 | −1.0 |
| TEP1 | 2.2 | 2.0 | −1.9 | 2.9 |
| BCL2 | −1.0 | 1.7 | −1.1 | 3.6 |
| HSPA1A | 2.2 | 1.6 | −3.1 | 2.1 |
| HSPA1B | 7.3 | 7.2 | −1.2 | 3.1 |
| DDC | −1.7 | −2.0 | 3.1 | −1.0 |
| SIRT1 | 1.6 | 2.1 | −1.7 | 1.9 |
| KRAS | 1.8 | 1.6 | −1.9 | −1.0 |
| SOD1 | 1.1 | 1.0 | −1.2 | 1.3 |
| HSPA1L | 1.9 | 2.4 | −1.6 | 2.7 |
| ACE | 2.9 | 3.6 | −1.5 | 4.7 |
| TP53 | 2.0 | 2.1 | −1.6 | 3.1 |
| SOD2 | 2.0 | 2.1 | −1.3 | 1.7 |
| CASP9 | 1.2 | 1.5 | −1.7 | 1.2 |
| CCL4L1 | −1.7 | −2.0 | 3.1 | −1.0 |
| GH1 | −1.7 | −2.0 | 3.1 | −1.0 |
| MAPK14 | 2.9 | 2.3 | 349.7 | 1.6 |
| NOS2 | −1.7 | −2.0 | 3.1 | −1.0 |
| CASP2 | 3.0 | 3.7 | −1.5 | 1.4 |
| NFKB1 | 1.8* | 1.8* | −2.9 | −1.1 |
| NOS1 | −1.7 | −2.0 | 3.1 | |
| NOS3 | −1.7 | −2.0 | 1.8 | |
| IL8 | 3.8 | 1.5 | −1.6 | |
| IL11 | 1.7 | 1.7 | −1.5 | |
| IL33 | −1.7 | −2.0 | 3.1 | |
| VEGFA | 3.2 | 3.7 | −1.2 | |
| FOS | 3.3 | 2.9 | −1.3 | |
| JUN | 4.5 | 5.3 | −1.6 | |
| MMP1 | 1.3 | 1.1 | −1.3 | |
| TIMP3 | −1.7 | −2.0 | 3.1 | |
| COL1A1 | 3.9 | 4.8 | 1.0 | |
| EGF | −1.7 | −2.0 | 3.1 | |
| EGR2 | 4.5 | 4.3 | −1.7 | |
| PDGFRL | 1.3 | 1.5 | −1.8 | |
| TGFB1 | 6.8 | 7.0 | −1.4 | |
| PARP3 | 3.0 | 3.1 | −1.7 | |
| PARP4 | 1.1 | 1.1 | −1.4 | |
| TPP1 | 1.1 | 1.1 | −1.6 | |
| POT1 | 1.3 | 1.4 | −2.4 | |
| RAP1A | 1.4 | 1.4 | −1.5 | |
| TERF2 | 3.6 | 2.7 | −1.4 | |
| TINF2 | 2.9 | 3.0 | −1.3 | |
| GPX1 | 1.0 | 1.0 | −1.4 | |
| SIRT2 | 4.2 | 3.7 | −1.3 | |
| SIRT4 | 1.4 | 1.1 | −1.7 | |
| KL | −1.7 | −2.0 | 3.1 | |
| PPARGC1A | −1.3 | −1.3 | 3.1 | |
| HSPA6 | −1.7 | −2.0 | 3.1 | |
| BCL2L1 | 6.6 | 5.9 | −1.3 | |
| FOXO3 | 3.1 | 3.0 | −2.1 | |
| HMOX1 | 4.5 | 2.2 | −1.9 | |
| TIMM22 | −1.2 | 1.1 | −1.4 | |
| TOMM40 | 4.5 | 4.8 | −1.3 | |
| SERPINB2 | −1.6 | −1.9 | 3.1 | |
| KIT | 2.3 | 2.9 | −1.8 | |
| NEIL1 | 2.4 | 2.8 | −1.8 | |
| CRP | −1.7 | −2.0 | 3.1 | |
| DUSP2 | −1.7 | −2.0 | 3.1 | |
| IMMP1L | −1.3 | −1.0 | −1.8 | |
| HBEGF | 1.9 | 2.5 | −1.2 | |
| SIRT3 | 2.6 | 3.0 | −1.5 | |
| CDKN2A | −1.1 | 1.1 | −2.4 | |
| BMP2 | −1.7 | −2.0 | 3.1 | |
| COL3A1 | 7.3 | 8.6 | −1.9 | |
| UBE2S | 1.5 | 1.7 | −1.5 | |
| GCH1 | 1.0 | 2.0 | 1.4 | |
| PARP9 | 1.8 | 1.9 | 2.7 | |
| S100A7 | −1.7 | −2.0 | 3.1 | |
| GAPDH | −1.0 | 1.0 | −1.1 | |
| ACTB | 1.5 | 1.8 | −1.3 | |
| HPRT1 | 1.1 | 1.1 | −2.2 | |
| HGDC | −1.7 | −2.0 | 3.1 | |

### Example 24

### Bioreactor for Growth & Maintenance of Coffee Berry Tissue Cultures

This example provides general procedural guidelines for using a CelliGen™ 115 benchtop bioreactor (New Brunswick Scientific) for the growth and maintenance of coffee berry tissue culture. Though it is believed these guidelines are generally applicable, it is recognized that the procedures and parameters will be modified—for instance to suit each plant or tissue type being cultured, each species or cultivar, and the output (metabolite production) that is desired. Elicitations also will be added as additional parameters that are tailored for specific situations.

The CelliGen™ 115 bioreactor will be set up and connected according to manufacturer's instructions; the impeller used is a pitched blade impeller in order to minimize shear stress on the cells (see Mirro & Voll, BioProcess International 7(1):52-57, 2009). Sterile cultured C. arabica cells (such as embryogenic cells grown on/in solid/liquid modified MS medium) at the early callus formation stage will be inoculated into the 3 L bioreactor at a ratios of 0.5 g Fresh Weight/L (or 1.5 g of embryogenic cells total). The temperature of the media in the bioreactor will be kept at 25° C. The speed of the impeller/rotor will be kept at 50 rpm, until day 21 where it will be increased to 100-120 rpm. The aeration rate will be 0.04 VVM (volume of air per medium volume per minute). The dissolved oxygen will always be over 30% and may be as high as 80% over the course of the run. The pH level will be kept at a neutral level (between 5.5 and 8.0). The feed rate will be between 0.02 and 0.2 g/L/hr. The bioreactor will be kept in the dark, or on an 8-10 hr light, 16-14 hr dark cycle.

The biomass is expected to reach maximum density (the point at which cell growth in the culture prevents sufficient mixing and aeration of the culture) at approximately 40-58 days post inoculation.

Metabolites, such as secondary metabolites, can be siphoned from the medium as the full 3 L volume should be replaced every about three days based on the slower feed rate. The metabolites could then be concentrated or extracting through biofractionation looking for specific compounds, etc., and then used in cell culture testing or incorporated into a test product.

At the close of the bioreactor cycle, the biomass will be removed and either: flash frozen and ground into powder, freeze dried and ground into powder, ground in an alcohol extraction process, dried at a constant low heat over time and ground into powder, etc., and then used in cell culture testing or incorporated into a test product.

### Example 25

### Extraction and Clinical Testing of Generated Product

This example describes a general plan for extracting and testing biological products (metabolites) from cultured cells.

Once a viable quantity of callus tissue from any of the cultured plants is ready, it is extracted (ground up dry, flash frozen and dried, sonicated, etc. . . . method pending) and the extract (or components of the extract) is tested on human skin fibroblasts in culture to determine efficacy and toxicity levels.

Various concentrations are placed into 96 well culture dishes containing human skin fibroblasts and an MTT assay is run to determine the amount (if any) of cell death occurs after a predetermined period (for instance 24 hours post extract incubation).

Following MTT testing, human skin fibroblasts are then subjected to a minimum three point dose response curve (starting with the concentration of extract that causes the lowest or no cell death) and decreasing logarithmically from the initial concentration. RNA is extracted from these cells 24 hrs post incubation with the extract and run on custom human RT-PCR arrays (using genes in Array 1 or Array 2, for instance). The data from this experiment is used to determine the best active concentration used for the clinical testing of the prototype product.

The selected product is safety and stability tested, and then used on a small (for instance, 10-20) subject pilot study to determine efficacy for photoaging effects.

When a successful product is identified, the extract itself can be further fractionated and analyzed to determine specific active compound in the extract. Mass production of specific active compounds can then be examined.

### Example 26

### Standard Operating Procedure (SOP) for Bioreactor

This example provides a representative and non-limiting procedure for producing and harvesting cells, biomass, and/or metabolite(s) from plant cell culture, using a benchtop bioreactor. Optionally, for some tissues or some embodiments (e.g., when elicitation is desired), one or more variables of the medium, or other growing conditions, may be modified from the SOP.

A BioFlo®/CelliGen® 115 Benchtop Fermentor and Bioreactor (New Brunswick Scientific, Edison, N.J.) is connected to nitrogen, compressed air, oxygen and carbon dioxide. The vessel is waterjacketed and cooled using a constant water supply. The vessel is prepared and sterilized according to manufacturers specifications and includes a Tri-Port, sample line and two addition lines if required. By way of example, a 3.0 L vessel is filled 1.25 L of liquid M527 Phytotechnology: Murashige Modified Multiplication Basal Medium. The temperature of the media in the bioreactor is maintained at 20-50° C.±5° C.

Sterile cultured callus or selected embryonic tissue sourced from at the early callus formation stage is opened under sterile conditions and finely chopped into pieces no larger than about 0.5 cm in diameter. The chopped callus tissue is weighed and placed into sterile liquid Murashige Modified Multiplication Basal Medium for inoculation into the bioreactor.

The speed of the impeller/rotor is kept at 30-100 rpm, until the biomass growth impedes the impeller process and then the speed is increased for instance to 120-1000 rpm. The aeration rate is initially 0.5-5.0 L/H, then increased based on proliferation of tissue in 1- 5 or 5-10 L/H increments during proliferation, until a maximum of 20-100 L/H is reached. The dissolved oxygen is typically maintained around 25% and may be as high as 80% over the course of the run. The pH level is typically kept at a 4.0-8.5±0.5 for the duration of the run.

25%-75% of the media is drained and replaced once every 2-8 weeks, increasing in frequency as the biomass increases until a maximum of 100% sterile media is replaced every 2-8 weeks.

The bioreactor is typically maintained in darkness 0-24 hours during the day. In various embodiments, including specifically light-based elicitations, light may be provided to the culture in the bioreactor for some or all of the day. Optionally, specific wavelengths of light may be specifically applied or not applied to cells within the bioreactor.

The biomass is expected to reach its maximum density (the point at which it prevents sufficient mixing and aeration of the culture) approximately 20-100 days, depending upon tissue source, growth conditions, and other variables.

Plant metabolites can be siphoned from the medium as the volumes of liquid media are replaced. The metabolites can then be concentrated or extracting through biofractionation and/or other processes to isolate specific compounds and then used in cell culture testing or incorporated into a commercial product.

At the close of the bioreactor cycle the biomass is typically removed and one of: flash frozen and ground into powder, freeze dried and ground into powder, ground in an alcohol extraction process, dried at a constant low heat over time and ground into powder, or processed by other methods known in the industry and then used in cell culture testing or incorporated into a test or commercial product.

### Example 27

### Carrot Callus Tissue in a Bioreactor

This example provides a representative and non-limiting procedure for producing and harvesting cells, biomass, and/or metabolite(s) from carrot callus cell culture, using a benchtop bioreactor.

A BioFlo®/CelliGen® 115 Benchtop Fermentor and Bioreactor (New Brunswick Scientific, Edison N.J.) is connected to Nitrogen, compressed air, Oxygen and Carbon Dioxide. The vessel is waterjacketed and cooled using a constant water supply. The 3.0 L vessel was filled 1.25 L of liquid Carrot Callus Initiation Medium (C212, Phytotechnology), after being prepared and sterilized according to manufacturer's specifications. The temperature of the media in the bioreactor is maintained at 25° C.±5° C. Sterile cultured carrot callus tissue {Duacus carota L. subsp. Sativus (Hoffm.)} sourced from Carolina Biological Supply (direct from supplier, 3 vials) at the early callus formation stage were opened under sterile conditions and finely chopped into pieces no larger than 0.5 cm in diameter. The chopped callus tissue was weighed and placed into sterile liquid Carrot Callus Initiation Medium for inoculation into the bioreactor.

The speed of the impeller/rotor was kept at 100 rpm; it would be increased to 120-150 rpm when the biomass growth impedes the impeller process. The aeration rate was initially 3 L/H, and could be increased based on proliferation of tissue in 10 L/H increments until a maximum of 40 L/H is reached.

The dissolved oxygen is typically maintained around 30% and may be as high as 80% over the course of the run. The pH level is typically kept at a 5.5±0.5 for the duration of the run.

25% of the sterile media could be drained and replaced once every 7-8 weeks, increasing in frequency as the biomass increases until a maximum of 50% sterile media every 4-6 weeks. The bioreactor is typically maintained in darkness, but may alternatively be maintained, or on a 6-8 hrs light, 18-16 hr dark cycle.

The biomass is expected to reach its maximum density (the point at which it prevents sufficient mixing and aeration of the culture) approximately 60-70 days.

Secondary metabolites can be siphoned from the medium as the volumes of liquid media are replaced (e.g., as discussed above). The metabolites can then be concentrated or extracting through biofractionation to isolate specific compounds and then used in cell culture testing or incorporated into a commercial product.

At the close of the bioreactor cycle the biomass is typically removed and either: flash frozen and ground into powder, freeze dried and ground into powder, ground in an alcohol extraction process, dried at a constant low heat over time and ground into powder, or processed by other methods known in the industry and then used in cell culture testing or incorporated into a test or commercial product.

Using the above procedure, carrot callus tissue was maintained in a BioFlo®/CelliGen® 115 Benchtop Fermentor and Bioreactor for period of one month, with an increase in mass (measured after the tissue was dried) that indicates callus growth.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method for identifying an agent that modulates lifespan of a cell, tissue, organ or organism, the method comprising:
contacting the cell, tissue, organ or organism with a non-animal extract or non-animal-derived composition;
assessing the influence of the extract or composition on lifespan of the cell, tissue, organ or organism; and
selecting the extract or composition as one that modulates lifespan if there is a measurable influence on lifespan of the cell, tissue, organ or organism contacted with the extract or composition in comparison to a corresponding cell, tissue, organ or organism not contacted with the extract or composition, thereby identifying the agent as one that modulates lifespan.

2. The method of claim 1, wherein the extract or composition is prepared from or derived from plant, fungus, algae, or bacterium cells.

3. The method of claim 2, wherein the plant, fungus, algae, or bacterium cells are genetically modified.

4. The method of claim 2, wherein the plant, fungus, algae, or bacterium cells are subjected to a mechanical, chemical, or biological elicitation event prior to preparation of the extract or composition.

5. The method of claim 4, wherein the elicitation event comprises one or more of contact with or exposure to: specific wavelength(s) of light; electromagnetic radiation electrical current/potential ionizing radiation high or low light intensity; nitrogen source limitation; carbon source limitation; phosphorus source limitation; water limitation; high salt exposure; high temperature exposure; low temperature exposure; contact stress or wounding; a pathogen-derived compound; a pesticide; a herbicide; a fungicide; a bactericide; anti-viral agent; wounding; a microbial (bacterial, viral, fungal) pathogen or fraction thereof; a nematode or fraction thereof; peroxide; an enzyme; a chemical; a fatty acid; an amino acid; saliva from herbivorous insect or other animal; vibration; gravity or lack thereof, or reduced or increased gravitational field; an extract from a plant; cAMP; ethylene or another gas; and/or a transformation vector (that results in expressing an eliciting compound or protein).

6. The method of claim 2, wherein the plant, fungus, or algae cells are grown in tissue culture prior to preparation of the extract or composition.

7. The method of claim 1, wherein the extract or composition is prepared from or derived from a plant of the family Rubiaceae, a plant of the family Theaceae, a plant of the family Orchidaceae, a plant of the family of Rosaceae, a microalgae, Coffea arabica, Camellia sinensis, Vaccinia species, Vaccinium macrocarpon, Vaccinium mebranaceum, Vaccinium fonnosum, Euterpe oleracea, Sequoiadendron giganteum, Sequoia sempervirens, Boswellia sacra, Fragaria virginiana, Vitis rotundifolia, Haematococcus pluvialis, a Phaffia yeast species, or another plant or other organism listed herein.

8. The method of claim 1, wherein the agent extends lifespan.

9. The method of claim 1, wherein the agent shortens lifespan.

10. The method of claim 1, wherein assessing the influence of the extract or composition on lifespan comprises determining if the extract or composition modulates activity or level of at least one telomere length maintenance gene.

11. The method of claim 10, wherein the telomerase length maintenance gene is selected from the group consisting of TERT, TERC, NRF2, POT1, TRF1, TRF2, TIN2, TPP1, RAP1, TNKS, TNKS 2, TERF2, TERF2IP, POLG, POLB, POLD3, POLE, POLI, POLL, PARP2, PPARG, SHC1, PTOP, IFI44, NFKB1, HSPA1A, HSPA1B, HSPA1L, MTND5, HPGD, IDH2, MDH1, MDH2, ME1, ME2, ME3, MTHD1, MTHFD1L, MTHFR, NADK, NADSYN1, NDUFA2, NDUFA3, NDUFA4, NDUFA4L2, NDUFA5, NDUFA6, NDUFA7, NDUFA9, NDUFA10, NDUFA12, NDUFB2, NDUFB3, NDUFB5, NDUFB6, NDUFB7, NDUFB8, NDUFB9, NDUFC2, NDUFS2, NDUFS4, NDUFS5, NDUFS7, NDUFS8, NDUFV2, NDUFV3, NOX1, NOX3, NOX4, NOX5, NOXA1, NOXO1, NQO1, FOXO1, FOXO3, FOXO4, LMNA, NHP2L1, RAD50, RAD51, KL and KU70.

12. The method of claim 1, wherein assessing the influence of the extract or composition on lifespan comprises determining if the composition modulates activity or level of at least one of:
(a) the genes listed as part of Array 1;
(b) the genes listed as part of Array 2;
(c) VEGFA, HMOX1, CCL4L1, DDC, NOS2A, SIRT1, TERT, PTGS2, or IFI44;
(d) four or more of TERT, TERC, NRF2, POT1, TRF1, TRF2, TIN2, TPP1, RAP1, TNKS, TNKS 2, TERF2, TERF2IP, POLG, POLB, POLD3, POLE, POLI, POLL, PARP2, PPARG, SHC1, PTOP, IF144, NFKB1, HSPA1A, HSPA1B, HSPA1L, MTND5, HPGD, IDH2, MDH1,MDH2, ME1, ME2, ME3, MTHD1, MTHFD1L, MTHFR, NADK, NADSYN1, NDUFA2, NDUFA3, NDUFA4, NDUFA4L2, NDUFA5, NDUFA6, NDUFA7, NDUFA9, NDUFA10, NDUFA12, NDUFB2, NDUFB3, NDUFB5, NDUFB6, NDUFB7, NDUFB8, NDUFB9, NDUFC2, NDUFS2, NDUFS4, NDUFS5, NDUFS7, NDUFS8, NDUFV2, NDUFV3, NOX1, NOX3, NOX4, NOX5, NOXA1, NOXO1, NQO1, FOXO1, FOXO3, FOXO4, LMNA, NHP2L1, RAD50, RAD51, KL and KU70;
(e) BCL2, SOD1, TP53, and SOD2;
(f) BCL2, SOD1, TP53, SOD2, BCL2L1, TIMM22, TOMM40, IMMP1L, CDKN2A, GAPDH, ACTB, HRP1, and HGDC;
(g) PARP1, PARP2, TERT, TEP1, TPS3, JUN, PARP3, PARP4, TERF2, TINF2, and CDKN2A;
(h) PARP1, PARP2, TERT, TEP1, and TP53;
(i) TERF2, POT1, TERT, and TPP1;
(j) PAPR1, PARP2, PARP3, and PARP4;
(k) PARP2, CYP19A1, TEP1, BCL2, HSPA1A, ACE, TP53, and NFKB1;
(l) IGF1, IGF2, PPARG, IL10, APOE, TERT, TNF, HLA-DRA, DDC, CCL4L1, NOS2A, and GH1;
(m) PARP1, IL6, SIRTT1, KRAS, and HSPA1L;
(n) IGF1, IL6, PPARG, IL10, TERT, TNF, TEP1, HSPA1A, SIRT1, TP53, GH1, NOS2A, and PPC; or
(o) a combination of two or more of (a) through (n).

13. The method of claim 1, wherein assessing the influence of the extract or composition on lifespan comprises determining if the extract or composition modulates mitochondrial regeneration, biosynthesis, proliferation, maintenance, or function.

14. The method of claim 13, wherein assessing the influence of the extract or composition on lifespan comprises determining if the extract or composition modulates activity or level of at least one of:
(a) the genes listed as part of Array 1;
(b) the genes listed as part of Array 2;
(c) VEGFA, HMOX1, CCL4L1, DDC, NOS2A, SIRT1, TERT, PTGS2, or IFI44;
(d) four or more of TERT, TERC, NRF2, POT1, TRF1, TRF2, TIN2, TPP1, RAP1, TNKS, TNKS 2, TERF2, TERF2IP, POLG, POLB, POLD3, POLE, POLI, POLI, PARP2, PPARG, SHC1, PTOP, IFI44, NFKB1, HSPA1A, HSPA1B, HSPA1L, MTND5, HPGD, IDH2, MDH1,MDH2, ME1, ME2, ME3, MTHD1, MTHFD1L, MTHFR, NADK, NADSYN1, NDUFA2, NDUFA3, NDUFA4, NDUFA4L2, NDUFA5, NDUFA6, NDUFA7, NDUFA9, NDUFA10, NDUFA12, NDUFB2, NDUFB3, NDUFB5, NDUFB6, NDUFB7, NDUFB8, NDUFB9, NDUFC2, NDUFS2, NDUFS4, NDUFS5, NDUFS7, NDUFS8, NDUFV2, NDUFV3, NOX1, NOX3, NOX4, NOX5, NOXA1, NOXO1, NQO1, FOXO1, FOXO3, FOXO4, LMNA, NHP2L1, RAD50, RAD51, KL and KU70;
(e) BCL2, SOD1, TP53, and SOD2;
(f) BCL2, SOD1, TP53, SOD2, BCL2L1, TIMM22, TOMM40, IMMP1L, CDKN2A, ACTB, HRP1, and HGDC;
(g) PARP1, PARP2, TERT, TEP1, TPS3, JUN, PARP3, PARP4, TERF2, TINF2, and CDKN2A;
(h) PARP1, PARP2, TERT, TEP1, and TP53;
(i) TERF2, POT1, TERT, and TPP1;
(j) PAPR1, PARP2, PARP3, and PARP4;
(k) PARP2, CYP19A1, TEP1, BCL2, HSPA1A, ACE, TP53, and NFKB1;
(l) IGF1, IGF2, PPARG, IL10, APOE, TERT, TNF, HLA-DRA, DDC, CCL4L1, NOS2A, and GH1;
(m) PARP1, IL6, SIRTT1, KRAS, and HSPA1L;
(n) IGF1, IL6, PPARG, IL10, TERT, TNF, TEP1, HSPA1A, SIRT1, TP53, GH1, NOS2A, and PPC; or
(o) a combination of two or more of (a) through (n).

15. The method of claim 1, wherein the method is carried out using a cell.

16. The method of claim 15, wherein the cell is in vitro.

17. The method of claim 15, wherein the cell is a mammalian cell or a plant cell.

18. The method of claim 15, wherein the cell is a stem cell.

19. The method of claim 15, wherein the cell is a eukaryotic cell.

20. The method of claim 15, wherein the cell is a prokaryotic cell.

21. The method of claim 1, wherein assessing the influence of the extract or composition on lifespan comprises determining if the extract or composition modulates oxidative DNA damage.

22. The method of claim 1, wherein the extract or composition comprises at least one active compound selected from the group consisting of idebenone or an analog or derivative thereof, (+) catechin, (−) epicatechin, procyanidin oligomers 2 through 18, procyanidin B-5, procyanidin B-2, procyanidin A-2, procyanidin C-1, chlorogenic acid, quinic acid, ferulic acid, caffeic acid, coffee cherry proanthocyanidins, EGCG (epigallocatechin-3-gallate), EGC (epigallocatechin), ECG (epicatechin-3-gallate), EC (epicatechin), GCG (gallocatechin gallate), GC (gallocatechin), C (catechin), CG (catechin gallate), viniferin, gnetin H, suffruticosol B, astaxanthin, β-carotene, lutein, canthaxanthin, or another compound referenced herein.

23. The method of claim 1, wherein the extract or composition comprises at least one active compound other than idebenone or an analog or derivative thereof, (+) catechin, (−) epicatechin, procyanidin oligomers 2 through 18, procyanidin B-5, procyanidin B-2, procyanidin A-2, procyanidin C-1, chlorogenic acid, quinic acid, ferulic acid, caffeic acid, coffee cherry proanthocyanidins, EGCG (epigallocatechin-3-gallate), EGC (epigallocatechin), ECG (epicatechin-3-gallate), EC (epicatechin), GCG (gallocatechin gallate), GC (gallocatechin), C (catechin), CG (catechin gallate), viniferin, gnetin H, suffruticosol B, astaxanthin, β-carotene, lutein, canthaxanthin, or another compound referenced herein.

24. A method of modulating the lifespan of a cell, tissue, organ or organism, comprising contacting the cell, tissue, organ or organism with at least one agent identified by the method of claim 1. (Original) The method of claim 24, wherein the agent is:
dissolved in oil;
dispersed in oil;
dispersed in alcohol;
dispersed in an aqueous medium;
homogenized in an aqueous medium;
encapsulated;
processed into dry material; or
a combination of two or more thereof.

26. The method of claim 24, wherein the agent is processed into dry material, and the form of the dry material is stabilized beadlets, powder, an encapsulated form, granule, or a combination of two or more thereof.

27. The method of claim 24 wherein the agent is formulated as a liquid, a liquid capsule, a solid capsule or a tablet.

28. The method of claim 24, wherein the agent is added to a food or beverage product.

29. A cosmetic preparation comprising at least one active component of an extract or composition identified by the method of claim 1.

30. The cosmetic preparation of claim 29, further comprising at least one additional active component.

31. The cosmetic preparation of claim 30, wherein the at least one additional active component comprises a carotenoid, an antioxidant, a vitamin, a second natural extract, a sunscreen agent, retinoic acid, retinol, an alpha or beta hydroxyl acid, or another compound or preparation recognized as providing protection to or improvement of skin, health, and/or longevity.

32. The cosmetic preparation of claim 29, formulated for topical application.
